(19) United States
(12) Patent Application Publication
(10) Pub. No.: US 2020/0368322 A1
(43) Pub. Date: Nov. 26, 2020

(54) METHODS OF IDENTIFYING AND USING AGENTS FOR TREATING DISEASES ASSOCIATED WITH INTESTINAL BARRIER DYSFUNCTION

(71) Applicant: Yeda Research and Development Co. Ltd., Rehovot (IL)

(72) Inventors: Benjamin GEIGER, Rehovot (IL); Eran ELINAV, Mazkeret Batya (IL); Inna GROSHEVA, Rehovot (IL)

(73) Assignee: Yeda Research and Development Co. Ltd., Rehovot (IL)

(21) Appl. No.: 16/986,473

(22) Filed: Aug. 6, 2020

Related U.S. Application Data
(63) Continuation of application No. PCT/IL2019/050148, filed on Feb. 6, 2019.
(60) Provisional application No. 62/666,169, filed on May 3, 2018.
(60) Provisional application No. 62/627,798, filed on Feb. 8, 2018.

(51) Int. Cl. A61K 38/20; A61K 38/19; A61K 38/18; A61K 38/08

(52) U.S. Cl. CPC A61K 38/2086; A61K 38/195; A61K 38/1825; A61K 45/06; A61K 38/1816; A61K 38/08; A61K 38/1875

(57) ABSTRACT

A method of treating a disease associated with intestinal barrier dysfunction in a subject is disclosed. The method comprises administering to the subject a therapeutically effective amount of an agent which downregulates the amount of glucose in intestinal cells, with the proviso that the disease is not Diabetes or obesity. Other agents for treating diseases associated with intestinal barrier dysfunction are also disclosed.

### RELATED APPLICATIONS

This application is a Continuation of PCT Patent Application No. PCT/IL2019/050148 having international filing date of Feb. 6, 2019, which claims the benefit of priority under 35 USC § 119(e) of U.S. Provisional Patent Application Nos. 62/666,169 filed on May 3, 2018 and 62/627,798 filed on Feb. 8, 2018. The contents of the above applications are all incorporated by reference as if fully set forth herein in their entirety.

### SEQUENCE LISTING STATEMENT

The ASCII file, entitled 83899SequenceListing.txt, created on Aug. 6, 2020, comprising 850 bytes, submitted concurrently with the filing of this application is incorporated herein by reference.

### FIELD AND BACKGROUND OF THE INVENTION

The present invention, in some embodiments thereof, relates to methods of identifying and using agents for treating diseases associated with intestinal barrier dysfunction and, more particularly, but not exclusively, to inflammatory bowel disease.

The obesity pandemic has reached alarming magnitudes, affecting more than 2 billion people worldwide and accounting for more than 3 million deaths per year. A poorly understood feature of the ‘metabolic syndrome’ is its association with dysfunctions of the intestinal barrier, leading to enhanced permeability and translocation of microbial molecules to the intestinal lamina propria and systemic circulation. This influx of immune-stimulatory microbial ligands into the vasculature, in turn, has been suggested to underlie the chronic inflammatory processes that are frequently observed in obesity and its complications, while entry of pathogens and pathobionts through an impaired barrier leads to an enhanced risk of infection in obese and diabetic individuals, particularly at mucosal sites. However, the mechanistic basis for barrier dysfunction accompanying the metabolic syndrome remains poorly understood. Beyond metabolic disease, enhanced intestinal permeability has also been linked with systemic inflammation in a variety of conditions, including cancer, neurodegeneration, and aging. Thus, there is an urgent scientific need to better define the molecular and cellular orchestrators and disruptors of intestinal barrier function, in order to devise strategies to counteract the detrimental systemic consequences of gut barrier alterations.

### SUMMARY OF THE INVENTION

According to an aspect of the present invention there is provided a method of treating a disease associated with intestinal barrier dysfunction in a subject in need thereof, the method comprising administering to the subject a therapeutically effective amount of an agent which downregulates the amount of glucose in intestinal cells, with the proviso that the disease is not Diabetes or obesity, thereby treating the disease associated with intestinal barrier dysfunction.

According to an aspect of the present invention there is provided a method of treating an inflammatory bowel disease of a subject in need thereof, the method comprising administering to the subject a therapeutically effective amount of an agent which downregulates the amount of glucose in intestinal cells, thereby treating the inflammatory bowel disease.

According to an aspect of the present invention there is provided a method of identifying agents useful for treating a disease associated with intestinal barrier dysfunction of a subject, the method comprising:

(a) contacting tight junctions of epithelial cells with a disruptor agent that promotes disruption or destabilization of tight junctions of epithelial cells; and

(b) contacting the tight junctions of epithelial cells with a test agent; and

(c) analyzing the effect of the test agent on the tight junctions of epithelial cells, wherein when the test agent prevents disruption of, or stabilizes the tight junctions, it is indicative of the test agent being useful for treating the disease associated with intestinal barrier dysfunction of the subject.

According to an aspect of the present invention there is provided a method of treating a disease associated with intestinal barrier dysfunction in a subject in need thereof, the method comprising:

(a) identifying an agent useful for treating the disease as described herein; and

(b) administering to the subject a therapeutically effective amount of the agent, thereby treating the disease associated with intestinal barrier dysfunction.

According to an aspect of the present invention there is provided a method of treating a disease associated with intestinal barrier dysfunction in a subject in need thereof, the method comprising administering to the subject a therapeutically effective amount of an agent which increases the amount and/or activity of at least one agent set forth in Table 2, thereby treating the disease associated with intestinal barrier dysfunction.

According to an aspect of the present invention there is provided a method of treating a disease associated with intestinal barrier dysfunction in a subject in need thereof, the method comprising administering to the subject a therapeutically effective amount of an agent which decreases the amount and/or activity of at least one agent set forth in Table 3, thereby treating the disease associated with intestinal barrier dysfunction.

According to an aspect of the present invention there is provided a co-culture system comprising cells of a tissue derived from a subject and microbes derived from a microbiome of the tissue of the subject.

According to an aspect of the present invention there is provided a method of treating a condition treatable by oral administration of a therapeutically active agent in a subject in need thereof, the method comprising administering to the subject at least one agent set forth in Table 3, thereby treating the condition.

According to an aspect of the present invention there is provided an agent which downregulates the amount of glucose in intestinal cells for use in treating a disease associated with intestinal barrier, with the proviso that the disease is not Diabetes or obesity.

According to an aspect of the present invention there is provided an agent which downregulates the amount of glucose in intestinal cells for use in treating an inflammatory bowel disease.

According to an aspect of the present invention there is provided an agent which increases the amount and/or activity of at least one agent set forth in Table 2 for use in treating a disease associated with intestinal barrier dysfunction.

According to an aspect of the present invention there is provided an agent, which decreases the amount and/or activity of at least one agent set forth in Table 3 for use in treating a disease associated with intestinal barrier dysfunction.

According to some embodiments of the invention, the subject has a comorbidity metabolic disease.

According to some embodiments of the invention, the metabolic disease is selected from the group consisting of obesity, diabetes, fatty liver disease and pre-diabetes.

According to some embodiments of the invention, the agent is administered for less than 1 month.

According to some embodiments of the invention, the subject is hyperglycemic.

According to some embodiments of the invention, the agent is an inhibitor of a glucose transporter.

According to some embodiments of the invention, the glucose transporter is GLUT2.

According to some embodiments of the invention, the inhibitor of the GLUT2 comprises a flavonoid.

According to some embodiments of the invention, the flavonoid comprises a flavonol.

According to some embodiments of the invention, the agent is an inhibitor of glucose metabolism.

According to some embodiments of the invention, the method is carried out in vitro or ex vivo.

According to some embodiments of the invention, the agent downregulates the amount of glucose in intestinal cells to a greater extent than the agent downregulates the amount of glucose in non-intestinal cells.

According to some embodiments of the invention, the agent that promotes disruption or destabilization is a sample derived from the GIT of the subject.

According to some embodiments of the invention, the test agent upregulates an amount or activity of a component of a sample derived from the gastrointestinal tract (GIT) of a subject having the disease, the component being present in an amount in the sample which is down-regulated compared to the amount in a sample derived from the GIT of a healthy subject.

According to some embodiments of the invention, the test agent downregulates an amount or activity of a component of a sample derived from the gastrointestinal tract (GIT) of a subject having the disease, the component being present in an amount in the sample which is up-regulated compared to the amount in a sample derived from the GIT of a healthy subject.

According to some embodiments of the invention, the agent which promotes disruption or destabilization of tight junctions of epithelial cells is set forth in Tables 5, 7 or 9.

According to some embodiments of the invention, the analyzing comprises quantitatively analyzing.

According to some embodiments of the invention, the epithelial cells are selected from the group consisting of CaCo-2 cells, DLD-1 cells, HT-29 cells, T-84 cells and LoVo cells.

According to some embodiments of the invention, the analyzing comprises analyzing the structure of the disrupted or destabilized tight junctions.

According to some embodiments of the invention, the analyzing is effected no more than three days following step (b).

According to some embodiments of the invention, the epithelial cells are seeded on an adhesive matrix.

According to some embodiments of the invention, the adhesive matrix comprises collagen, fibronectin or Matrigel.

According to some embodiments of the invention, the sample is a fecal sample.

According to some embodiments of the invention, the sample is an intestinal mucosal sample.

According to some embodiments of the invention, the sample comprises bacteria.

According to some embodiments of the invention, the analyzing the structure of the disrupted or destabilized tight junctions comprises analyzing expression of at least one marker of tight junctions.

According to some embodiments of the invention, the marker is selected from the group consisting of cingulin or ZO-1.

According to some embodiments of the invention, the method further comprises analyzing expression of at least one marker of focal adhesions.

According to some embodiments of the invention, the at least one marker of focal adhesions is selected from the group consisting of paxillin or zyxin.

According to some embodiments of the invention, the method further comprises analyzing expression of a nuclear marker.

According to some embodiments of the invention, the epithelial cells are derived from the subject.

According to some embodiments of the invention, the disease associated with intestinal barrier dysfunction is an infectious disease.

According to some embodiments of the invention, the disease associated with intestinal barrier dysfunction is a disease associated with systemic inflammation.

According to some embodiments of the invention, the disease associated with barrier dysfunction is selected from the group consisting of Non Alcoholic Fatty Liver Disease (NAFLD); Non alcoholic steatohepatitis (NASH); Pre-diabetes and glucose intolerance.

According to some embodiments of the invention, the disease associated with systemic inflammation is selected from the group consisting of cancer, aging and neurodegeneration.

According to some embodiments of the invention, the disease is a disease of the GIT.

According to some embodiments of the invention, the disease of the GIT is selected from the group consisting of inflammatory bowel disease, metabolic syndrome, gut infection and gut autoinflammation.

According to some embodiments of the invention, the inflammatory bowel disease (IBD) is Crohn's disease or ulcerative colitis.

According to some embodiments of the invention, the administering comprises orally administering.

According to some embodiments of the invention, the administering comprises co-administering the therapeutically active agent with the at least one agent set forth in Table 3.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-N. Obesity is associated with intestinal barrier dysfunction and enteric infection. (A-C) PRR stimulation by sera (A) and splenic (B) and hepatic extracts (C) from db/db mice. (D, E) ZO-1 staining (D) and quantification (E) of colonic sections from db/db mice and WT littermates. Scale bars, 100 μm. (F) FITC-dextran recovered from the serum of db/db mice and WT littermates after oral gavage. (G) Ussing chamber recording of colons from db/db mice and controls. (H-L) Abdominal luminescence (H, I) and CFUs recovered from mesenteric lymph nodes (J), spleens (K), and livers (L) from db/db mice infected with C. rodentium. (M, N) Total abdominal luminescence (M) and epithelial-adherent colonies (N) of C. rodentium in bone marrow chimeras of db/db and WT mice. All data represent at least two independent experiments. Means±SEM are plotted. * p<0.05, ** p<0.01, **** p<0.0001 by ANOVA (N) or Mann-Whitney U-test (all other panels).

FIGS. 2A-O. Obesity does not suffice to explain susceptibility to enteric infection. (A) PRR stimulation by splenic extracts from mice fed a high-fat diet (HFD). (B-E) Abdominal luminescence (B) and CFUs recovered from colonic tissue (C), spleens (D) and livers (E) of HFD-fed mice infected with luminescent C. rodentium. (F-H) Body weight (F), C. rodentium luminescence (G), and C. rodentium-induced mortality (H) in paired-fed db/db mice and controls. (I-L) Total abdominal luminescence signals (I) and live CFUs recovered from colonic tissue (J), mesenteric lymph nodes (K) and livers (L) from leptin antagonist (LeptAnt)-treated mice infected with bioluminescent C. rodentium. (M-O) Blood glucose levels in paired-fed db/db mice (M), HFD-fed mice (N) and LeptAnt-treated mice (O). All data represent at least two independent experiments. Means±SEM are plotted. * p<0.05, ** p<0.01, *** p<0.001, **** p<0.0001 by ANOVA (F, M) or Mann-Whitney U-test (all other panels).

FIGS. 3A-N. Hyperglycemia causes susceptibility to enteric infection. (A-E) Abdominal luminescence (A, B) and CFUs recovered from colonic tissue (C), spleens (D) and livers (E) from STZ-treated mice infected with bioluminescent C. rodentium. (F, G) E-cadherin staining (F) and quantification (G) of colons from STZ-treated mice and controls. Scale bars, 25 μm. (H) Ussing chamber recordings from colons of STZ-treated mice and controls. (I) FITC-dextran recovered from the serum of STZ-treated mice after oral gavage. (J) Detection of 16S rDNA in livers of STZ- and Abx-treated mice. (K, L) PRR stimulation by sera (K) and hepatic extracts (L) from STZ-treated mice and controls, with or without antibiotic (Abx) treatment. (M, N) Abdominal luminescence (M) and hepatic CFUs (N) from C. rodentium-infected Akita mice. All data represent at least two independent experiments. Means±SEM are plotted. * p<0.05, *** p<0.001, **** p<0.0001 by ANOVA (J) or Mann-Whitney U-test (all other panels).

FIGS. 4A-L. Hyperglycemia alters intestinal epithelial cell function. (A, B) Colonic E-cadherin intensity (A) and PRR ligand stimulation by sera (B) from STZ-treated mice and controls, with or without insulin administration. Scale bars, 25 μm. (C, D) Abdominal luminescence (C) and CFUs recovered from the spleen (D) of STZ- and insulin-treated mice after C. rodentium infection. (E-H) Quantification of barrier tortuosity (E, G) and representative ZO-1 staining (F, H) of Caco-2 cells treated with different concentrations and exposure times of glucose. Scale bars, 10 μm. (I-K) PCA (I), heatmap (J) and KEGG pathway annotation (K) of differentially expressed genes in the epithelium of STZ-treated mice and controls. (L) Differentially expressed genes contributing to N-glycan biosynthesis. All data represent at least two independent experiments. Means±SEM are plotted. n.s. not significant, ** p<0.01, **** p<0.001 by ANOVA.

FIGS. 5A-L. Epithelial reprogramming by hyperglycemia involves glucose metabolism and GLUT2. (A-C) Quantification of barrier tortuosity (A, C) and representative ZO-1 staining (B) of Caco-2 cells treated with the indicated concentrations of glucose and 2-deoxyglucose (2-DG). Scale bars, 10 μm. (D) Similarity matrix of the epithelial transcriptomes of STZ-treated mice, with or without 2-DG administration. (E, F) PRR stimulation by hepatic extracts (E) and sera (F) from STZ-treated mice, with or without 2-DG administration. (G, H) Splenic CFUs from C. rodentium-infected and 2-DG-treated STZ (G) and db/db mice (H). (I-K) Colonic ZO-1 (I) and E-cadherin intensity (J) and PRR stimulation by hepatic extracts (K) from STZ-treated GLUT2^{ΔIEC} mice and controls. (L) CFUs recovered from spleens of STZ-treated GLUT2^{ΔIEC} mice and controls infected with C. rodentium. All data represent at least two independent experiments. Means±SEM are plotted. n.s. not significant, * p<0.05, ** p<0.01, *** p<0.001, **** p<0.0001 by ANOVA.

FIGS. 6A-C. Hyperglycemia is associated with influx of microbial products in humans. (A, B) Correlation matrix (A) and average correlations with systemic PRR ligands (B) of the indicated parameters in the serum of 27 healthy volunteers. (C) Correlation of HbA1c with serum levels of TLR4 ligands.

FIGS. 7A-O. Obesity predisposes to intestinal barrier dysfunction. (A) Body weight of 9-week old db/db mice and controls. (B, C) PRR stimulation by sera (B) and liver extracts (C) from db/db mice. (D) PCA of colonic gene expression in db/db mice and WT littermates. (E) Heatmap of differentially expressed genes in the colons of db/db mice and WT littermates. (F) Expression of genes related to tight junction formation in the colons of db/db mice and WT littermates. (G-N) Representative colonic luminescence (G, M), quantification of colonic luminescence (H, N), representative luminescence from peripheral organs (I), and abdominal luminescence (J, K) from C. rodentium-infected db/db (G-I) and ob/ob mice (J-N). (0) CFUs in mesenteric lymph nodes of C. rodentium in bone marrow chimeras of db/db and WT mice. Means±SEM are plotted. n.s. not significant, * p<0.05, ** p<0.01, *** p<0.001, by ANOVA (0) or Mann-Whitney U-test (all other panels).

FIGS. 8A-O. Analysis of LepR-expressing populations involved in intestinal host defense. Abdominal luminescence measurements after C. rodentium infection of the indicated conditional LepR-deficient mice. The three experiments in G-O were gender- and vivarium-, but not age-matched. Means±SEM are plotted. n.s. not significant, * p<0.05, **** p<0.0001 by Mann-Whitney U-test.

FIGS. 9A-O. Analysis of CNS LepR-expressing populations involved in intestinal host defense. Abdominal luminescence measurements after C. rodentium infection of the indicated conditional LepR-deficient mice. n.s. not significant by Mann-Whitney U-test.

FIGS. 10A-J. Obesity per se does not explain susceptibility to enteric infection. (A) Body weight development of mice fed a high-fat diet (HFD). (B) CFUs recovered from mesenteric lymph nodes from HFD-fed mice infected with C. rodentium. (C) Body weight development of mice treated with leptin antagonist (LeptAnt). (D-F) Representative abdominal luminescence (D), CFUs recovered from spleens (E) and representative luminescence recordings from internal organs (F) of LeptAnt-treated mice infected with C. rodentium. (G-J) Serum glucose levels of the indicated conditional LepR-deficient mice. The three experiments in G-I correspond to FIGS. 2G-O. Means±SEM are plotted. n.s. not significant, * p<0.05, ** p<0.01 by Mann-Whitney U-test.

FIGS. 11A-E. Hyperglycemia predisposes to intestinal barrier dysfunction. Serum glucose levels (A), PRR stimulation by livers (B) and sera (C), 16S rDNA quantification in spleens (D) and intestinal lumen (E) from STZ-treated mice and controls. Means±SEM are plotted. n.s. not significant, * p<0.05 by Mann-Whitney U-test.

FIGS. 12A-J. Dysbiosis does not predispose to intestinal barrier dysfunction. (A-D) PCoA plots (A, C) and UniFrac distances (B, D) of microbiota samples from STZ-treated mice and controls, with or without additional insulin treatment. (E) PRR stimulation by the indicated tissues from germ-free mice receiving microbiota from either STZ-treated donors or controls. (F-J) Tissue CFUs (F-I) and tissue-adherent colonic luminescence (J) from C. rodentium-infected germ-free mice receiving microbiota from either STZ-treated donors or controls. Means±SEM are plotted. n.s. not significant, * p<0.05, *** p<0.001, **** p<0.0001 by ANOVA (D) or Mann-Whitney U-test (all other panels).

FIGS. 13A-E. Additional evidence for hyperglycemia-induced barrier dysfunction. (A-C) Serum glucose levels (A), splenic CFUs (B) and representative luminescence recorded from spleens and livers (C) of C. rodentium-infected Akita mice. (D) Serum glucose levels of STZ-treated mice, with or without insulin administration. (E) Representative E-cadherin staining of STZ-treated mice, with or without insulin administration. Scale bars, 25 μm. Means±SEM are plotted. * p<0.05, ** p<0.01 by ANOVA (D) or Mann-Whitney U-test (A).

FIG. 14 The impact of hyperglycemia on intestinal epithelial cell function. RNA-sequencing results obtained from intestinal epithelial cells from STZ-treated mice and controls are shown in a pathway schematic of N-glycan biosynthesis.

FIGS. 15A-D. Hyperglycemia does not alter epithelial turnover. (A, B) Ki67 staining (A) and quantification (B) of intestinal tissue obtained from STZ-treated mice and controls. Scale bars, 100 μm. (C, D) Representative flow cytometry recording (C) and cell viability (D) of intestinal epithelial cells isolated from STZ-treated mice and controls. Means±SEM are plotted. n.s. not significant by Mann-Whitney U-test.

FIGS. 16A-J. The impact of hyperglycemia on immune cell populations. Relative abundance of the indicated immune cell populations in the colonic lamina propria (A-E) and spleens (F-J) from STZ-treated mice and controls. Means±SEM are plotted. n.s. not significant, * p<0.05 by Mann-Whitney U-test.

FIGS. 17A-H. The impact of hyperglycemia on IL-22-dependent immunity. (A) H&E staining of colonic tissue from STZ-treated mice and controls. Scale bars, 200 μm. (B-E) Gene expression of the indicated cytokines in colonic tissue from STZ-treated mice and controls. (F) Heatmap of gene expression in intestinal epithelial cells from STZ-treated mice and controls. (G, H) Representative whole body luminescence at early states of infection (G) and mortality (H) of C. rodentium-infected STZ-treated IL-22-deficient mice and controls. Means±SEM are plotted. n.s. not significant by Mann-Whitney U-test.

FIGS. 18A-J. Comparison of different infection routes in hyperglycemic mice. (A-E) CFU of Salmonella Typhimurium at the indicated tissues after oral infection. (F-J) CFU of Salmonella Typhimurium at the indicated tissues after systemic infection. Means±SEM are plotted. n.s. not significant. * p<0.05, ** p<0.01 by Mann-Whitney U-test.

FIGS. 19A-I. The impact of 2-deoxyglucose (2-DG) on epithelial function. (A) Quantification of intestinal epithelial metabolites involved in glycolysis. (B, C) Similarity quantification of the epithelial transcriptome (B) and expression of Alg8 in colonic epithelial cells (C) from STZ-treated mice and controls, with or without additional 2-DG treatment. (D-G) Abdominal luminescence (D) and bacterial CFUs recovered from the indicated organs (E, F) and from luminal content (G) of STZ-treated C. rodentium-infected mice, with or without additional 2-DG treatment. (H, I) Representative luminescence recording (H) and quantification (I) of bacterial CFUs recovered from the livers of C. rodentium-infected db/db mice, with or without additional 2-DG treatment. Means±SEM are plotted. n.s. not significant. * p<0.05, ** p<0.01 by Mann-Whitney U-test (A) or ANOVA (all other panels).

FIGS. 20A-I. GLUT2 mediates hyperglycemia-induced epithelial barrier dysfunction. (A) PCA of epithelial gene expression in STZ-treated GLUT2^{ΔIEC} mice and controls. (B) Heatmap of differentially expressed genes in the epithelium of in STZ-treated GLUT2^{ΔIEC} mice and controls. (C, D) ZO-1 (C) and E-cadherin staining (D) of colonic tissue from STZ-treated GLUT2^{ΔIEC} mice and controls. Scale bars, 25 μm. (E) Ussing chamber recording of colon tissue from STZ-treated GLUT2^{ΔIEC} mice and controls. (F) Serum glucose levels in STZ-treated GLUT2^{ΔIEC} mice and controls. (G-I) Total body luminescence (G) and CFUs recovered from the liver (H) and mesenteric lymph nodes (H) from C. rodentium-infected STZ-treated GLUT2^{ΔIEC} mice and controls. Means±SEM are plotted. * p<0.05, ** p<0.01 by ANOVA.

FIGS. 21A-G. Correlation between glycemic control levels and influx of microbial products in humans. (A, B) Age (A) and BMI (B) distribution in the study cohort. (C-G) Correlations between the indicated serum parameters (C-E) and fecal 16S molecules (G) with HbA1c (C-E, G) and BMI (F).

FIG. 22. Schematic of model for hyperglycemia-mediated barrier disruption.

FIG. 23. Caco2 (and related) cells as a model to study intestinal epithelium barrier function in IBD.

Upper panel: Non-treated Caco2 cells and cells treated with known pro-inflammatory IBD mediators (50 ng/ml TNFa, 30 ng/ml IL1-b, 100 ng/ml LPS) were fixed and stained for cingulum (green) to visualize apical tight junctions. All pro-inflammatory molecules tested cause significant alteration in tight junctions morphology.

Bottom panel: Caco2 cells treated with 100 ng/ml of IL-22 (non-inflammatory cytokine), in addition to the indicated treatment with pro-inflammatory agents. IL-22 completely restored normal tight junction morphology for each of the three disruptive agents.

FIG. 24. Bacterial metabolites which were found to be increased in dysbiotic mouse intestine cause tight junction disruption and increase of focal adhesion. Caco2 cells were treated with a set of bacterial metabolites previously shown to be decreased in dysbiotic mouse intestine (Levy et al., Cell. 2015 Dec. 3; 163(6): 1428-1443) and labeled with antibodies against cingulin and paxilin to visualize tight junctions and focal adhesions. Each of the tested metabolites demonstrated distortion of tight junction morphology. At the same time these metabolites also caused increase of focal adhesions size. Bacterial metabolites were used in following concentrations: 10 mM of acetyl-proline, 1 mM of putrescine, 10 mM of histamine, 5 mM of spermine.

FIG. 25. Epithelial disruptors and stabilizers identified in in the screening of human secreted molecules library. Caco2 cell were cultured for 24 hours in the presence of human secreted molecules, fixed and stained with anti-cingulin antibodies for tight junctions visualization. Out of 297 tested molecules, 11 caused changes in TJ morphology similar to previously observed upon treatment with known pro-inflammatory mediators. 4 treatments resulted in improvement of TJ morphology in comparison to non-treated control (bottom panel) and were recognized as potential epithelial stabilizers. The following concentrations of secreted molecules were used: TNFa-50 ng/ml, Il-15-25 ng/ml, CCL-20-50 ng/ml, CCL-23-50 ng/ml, FGF-1-25 ng/ml, FGF-10-100 ng/ml, BMP-10-50 ng/ml, UTS-2-10 ng/ml, UCN-1-20 ng/m, EPO-10 ng/ml, UCN-3-20 ng/ml, IL-21-30 ng/ml, CCL-3-40 ng/ml, TIMP-2-100 ng/ml, FasLG-10 ng/ml.

FIGS. 26A-B. Quantitate analysis of changes in TJ morphology upon treatment with selected disrupted or stabilizing molecules from human secreted molecules library. Tortuosity was measured as a ration between physical length of single junction and Euclidean distance between its endpoints. Epithelial disrupting agents cause significant increase of junctions tortuosity, while epithelial stabilizers bring it down to almost perfectly straight line. (N≥200).

FIG. 27. Effect of epithelial disruptors and stabilizers on cell-matrix focal adhesions. Same experiment as in FIG. 25. Cells were analyzed using anti-cingulin and anti-paxilin antibodies (in order to visualize cell-matrix focal adhesions). Images of focal adhesions correspond to the same fields of view as ones for cingulin but were taken at the different focal plane. Note, that in most of the cases treatments resulting in disruption of tight junctions simultaneously caused enlargement of focal adhesions. In contrast, tight junction stabilizing molecules induced reduction of focal adhesions size and increase in their number.

FIG. 28. Bacterial metabolites are capable of tight junction stabilization. Caco2 cells were treated with a set of bacterial metabolites previously shown to be decreased in dysbiotic mouse intestine alone and in combination with bacterial LPS in order to test their capacity to restore tight junctions visualized by cingulin staining. Three out of 14 tested metabolites demonstrated the ability to abolish tight junction distortion caused by LPS treatment alone. Bacterial metabolites were used in following concentrations: 10 mM of taurine, 1 mM of tryptamine, 10 mM of L-homo-serine.

FIG. 29. Stabilizing bacterial metabolites are capable of reduction of focal adhesions size. Same experiment as for FIG. 28. Focal adhesions visualized any paxilin staining in the same fields of view for which cingulin staining is presented. Note, that two stabilizing metabolites tryptamine and L-homo-serine caused reduction in focal adhesions size in comparison to control non treated cells and are also capable to prevent enlargement focal adhesions caused by LPS treatment.

FIG. 30. Design of drug library screening aiming to identify novel IBD therapeutics. Disease-mimicking conditions were created by Caco2 cells treatment with combination of LPS and histamine (bacterial antigen and bacterial metabolite). Characteristic changes in tight junction morphology were seen upon these combined treatments. Cells treated with pharmacologically active compounds from the drug library were visually examined for restoration of normal tight junction phenotype.

FIG. 31 shows selected examples of pharmacologically active compounds exhibiting ability to restore damaged tight junctions at 10 μM concentration.

FIG. 32 shows selected examples of pharmacologically active compounds disruptive activity on epithelial tight junctions.

FIG. 33 illustrates how selected stabilizers are capable of preventing disrupting effect of known pro-inflammatory agents.

FIG. 34 illustrates that IL-21 is capable of preventing disrupting effect of novel disruptive agents.

FIG. 35. Quantification of IL-21 effect on TJ morphology upon co-treatment with disruptive agents. Scatter plot presenting TJ tortuosity measured for the experiment described for FIG. 34 (N≥200). It can be seen that combination of disruptive agents with IL-21 reduces tortuosity to the level of untreated control.

FIGS. 36A-B. Quantification of tight junctions tortuosity upon cell treatment with combinations of all novel epithelial disruptors with all novel stabilizers. FIG. 36A: delta mean tortuosity (deviation from 1) for all single and combined treatments presented in the heat-map format. FIG. 36B: statistical significance of the difference between control sample and each treatment quantified via measurement of Earth Mover's Distance, log p-values for each treatment are presented in the heat-map format. Note, that all individual treatment with disruptors and stabilizers results in the significant change in junctions tortuosity, while most of the tested combinations (disruptor+stabilizers) bring the tortuosity values back to the control level.

FIG. 37. Epithelial disruption correlates with formation of apical actin-enriched structures. Caco2 cells were either left untreated or treated with 10 mM histamine, fixed and labeled for ZO-1 to visualize tight junctions and for actin. It can be seen that histamine treatment in addition to distortion of tight junctions geometry also causes assembly of actin fibers in the same plane where tight junctions are located. These structures are not seen in non-treated cells.

FIG. 38. Inhibition of acto-myosin contractility prevents epithelial disruption. Caco2 cells were treated with either LPS or histamine alone or in combination with 20 μM blebbistatin and labeled for cingulin and paxilin to visualize tight junctions and focal adhesions. Note, that co-treatment with blebbistain abolishes disruptive effect of LPS and histamine on tight junctions and simultaneously causes reduction of focal adhesions which are enlarged upon disruptor only treatments.

FIG. 39. Epithelial disruptors increase, and epithelial stabilizers decrease forces applied to the substrate. Sparse islands of Caco2 cells were grown on the polyacrylamide hydrogels with embedded fluorescent beads. Upon 24 hours treatment with indicated compounds live cells and substrate beneath them were imaged, then cells were detached by trypsinization and substrate was imaged in relaxed state. Beads displacement was analyzed and used for quantification of forces applied to the substrate. Note, that epithelial disruptors histamine increases cell contractility, while stabilizer tryptamine as well as known contractility inhibitor blebbistatin cause cell relaxation.

FIG. 40. Average traction forces appalled by Caco2 cells upon indicated treatments. Data from at least 2 independent experiments are presented.

FIG. 41. Fecal extract from healthy mouse is capable of restoring epithelial disruption caused by bacterial metabolites or antigens. Caco2 cells were treated either with histamine or LPS alone or in combination with aqueous extract from healthy mouse feces enriched with stabilizing metabolites. Combination of histamine with LPS resulted in improvement of tight junction morphology in comparison with cells treated with disruptive agents alone.

FIG. 42. Dysbiotic fecal extract possess disrupting activity, which can be neutralized by stabilizing metabolites or by the fecal extract from healthy animals. Caco2 cells were treated either with aqueous fecal extract obtained from dysbioyic Ask knock-out mice alone or in combination with 1 mM tryptamine or fecal extract from healthy animals. Note, that Ask−/− extract causes tight junctions distortion similarly to LPS and other type 1 disruptors, and this effect is neutralized by either stabilizing bacterial metabolite tryptamine or with fecal extract from healthy animals.

FIGS. 43A-B. Newly identified stabilizing bacterial metabolites are capable of abolishing the disruptive effect of broad spectrum of host and microbiota-derived disruptors. Quantification of tight junctions tortuosity upon treatment with either disruptors and stabilizers alone or in combinations. 43A: delta mean tortuosity (deviation from 1) for all single and combined treatments presented in the heat-map format. 43B: statistical significance of the difference between control sample and each treatment quantified via measurement of Earth Mover's Distance, p-values for each treatment are presented in the heat-map format. Note, that most of the tested combinations (disruptor+stabilizers) bring the tortuosity values back to the control level.

FIG. 44. Simultaneous treatment with type 1 and 2 disruptors results in combined tight junction phenotype. Caco2 cells were treated with either histamine or spermine alone or in combination with LPS. Histamine and spermine induce dramatic change in epithelial geometry and “flower-like” phenotype. Combination of either histamine or spermine with LPS caused a double effect—change in cell geometry (type 2 disruption) and appearance of zigzag shaped tight junctions (type 1 disruption).

FIG. 45. Disruptive effect of histamine on epithelial tight junctions is transduced via type 2 histamine receptor. Caco2 cells were treated with either histamine alone or in combination with anti-histamine drugs specific to different histamine receptors. Ranitidine—H2 receptor antagonist prevents disruptive effect of histamine on tight junction morphology.

FIGS. 46A-B. Panoramic view of CaCo2 cells with no treatment (Normal epithelium, 46A) and after perturbation (46B).

FIGS. 47A-B illustrate the reversible effect of the epithelial tight junction disruptors on Caco2 cells.

FIGS. 48A-J illustrate the disruptive effect of putrescine in mice with DSS-induced colitis.

(A-D) Wild-type mice with or without oral gavage of putrescine were administered with 2% DSS in the drinking water for 5 days (N=8 mice in each group).

(A) FITC-dextran levels recovered from the serum 3 hr after oral gavage (80 mg/ml FITC-dextran) on day 5 after DSS administration. (B) Ussing chamber recording of short circuit current (Isc) across colon epithelial layer on day 5 after DSS administration. (C) Ussing chamber recording of trans-epithelial electrical resistance across colon epithelial layer on day 5 after DSS administration. (D) PRR stimulation by lymph node extracts from mice on day 5 after DSS administration.

(E-J) Acute DSS colitis (2% DSS in drinking water for 7 days) was induced in WT mice with or without oral gavage of putrescine (n=8-10 mice in each group). Weight loss (E), survival (F), representative colonoscopy images on day 7(G), colonoscopy severity score on day 7 (H), representative histology images on day 10 (I), and pathology scoring on day 10 (J).

FIGS. 49A-N. Disruptive effect of putrescine in mice with C. rodentium infection.

(A-N) Wild-type mice with or without oral gavage of putrescine were infected with C. rodentium (n=8-15 mice each group).

(A-C) CFUs recovered from stool (A), abdominal bioluminescence quantification (B) and imaging (C) during the post-infection course.

(D-F) ex vivo colonic bioluminescence imaging (D) and quantification (E) and CFUs recovered from colonic tissue (F) at day 8 post infection.

(G-H) CFUs recovered from spleens (G), livers (H) and lymph nodes (I) at day 8 post infection.

(J-L) FITC-dextran levels recovered from the serum 3 hr after oral gavage (80 mg/ml FITC-dextran) on day 5 (J), Ussing chamber recording of short circuit current (Isc) (K) and trans-epithelial electrical resistance across colon epithelial layer (L) on day 8 post infection.

(M-N) ZO-1 staining (M) and quantification (N) of colonic sections at day 8 post infection.

FIGS. 50A-K. Restoration of the disruptive effect of putrescine by taurine supplement in mice with C. rodentium infection.

(A-K) Wild-type mice treated with vehicle control, putrescine, putrescine plus taurine were infected with C. rodentium (results were pooled from 3 independent experiments).

(A-C) CFUs recovered from stool (A), abdominal bioluminescence quantification (B) and imaging (C) during the post-infection course.

(D-F) CFUs recovered from colonic tissue (D), ex vivo colonic bioluminescence imaging (E) and quantification (F) and at day 7 post infection.

(G-H) CFUs recovered from spleens (G), livers (H) and lymph nodes (I) at day 7 post infection.

(J-K) Ussing chamber recording of short circuit current (Isc) (J) and trans-epithelial electrical resistance across colon epithelial layer (K) on day 7 post infection.

FIGS. 51A-D illustrates the effect of putrescine on TJ integrity in an intestinal organ culture system.

(A) Pictures showing the ex vivo three-dimensional colon culture system used for the perfusion of different concentrations of putrescine through colon lumens.

(B) Representative histology images of ex vivo colon tissues cultured with different concentrations of putrescine solution for 2 hours.

(C-D) ZO-1 staining (C) and quantification (D) of ex vivo colon tissues cultured with and without putrescine (33.2 mM).

FIGS. 52A-H illustrates the disruptive effect of putrescine in mice with DSS-induced colitis. (A-D) Wild-type mice with or without oral gavage of putrescine were administered with 2% DSS in the drinking water for 5 days (N=8 mice in each group). (A) Schematic illustration demonstrating setting of the experiment. (B) Weight changes after DSS administration until day 5. (C-D) PRR stimulation by spleen (C) and liver (D) extracts from mice on day 5 after DSS administration. (E-H) Acute DSS colitis (2% DSS in drinking water for 7 days) was induced in WT mice with or without oral gavage of putrescine (n=8-10 mice in each group). (E) Schematic illustration demonstrating setting of the experiment. (F) comparison of daily DSS consumption between groups. (G-H) measurement of colon lengths on day 10 after DSS administration.

FIGS. 53A-J illustrate the disruptive effect of putrescine in mice with C. rodentium infection.

(A-J) Wild-type mice with or without oral gavage of putrescine were infected with C. rodentium (n=8-15 mice each group).

(A) Schematic illustration demonstrating setting of the experiment. (B-E) Ki-67 staining (B) and quantification (C), cleaved caspase 3 staining (D) and quantification (E) of colonic sections at day 8 post infection. (F-J) Flow cytometry enumeration of Th17 (RORgt+) subsets of hematopoietic cells (F), secretion of inflammatory cytokine IL-22 (G-H), IL-17 and Interferon-g (I-J) harvested from the lamina propria of mice at day 8 post infection.

### DESCRIPTION OF SPECIFIC EMBODIMENTS OF THE INVENTION

The present invention, in some embodiments thereof, relates to methods of identifying and using agents for treating diseases associated with intestinal barrier dysfunction and, more particularly, but not exclusively, to inflammatory bowel disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Serum glucose is among the most strictly controlled physiological variables of organismal homeostasis. Chronically elevated glucose levels, as observed in diabetes mellitus, obesity and associated metabolic disorders, such as non-alcoholic fatty liver disease, result from altered homeostatic set points of the tightly regulated normoglycemic levels. Longstanding hyperglycemia, in turn, leads to a myriad of potentially devastating biochemical and physiological consequences, such as the generation of advanced glycation end products, pancreatic glucose toxicity, macrovascular and microvascular complications impacting virtually every organ, risk of infection, and enhanced mortality.

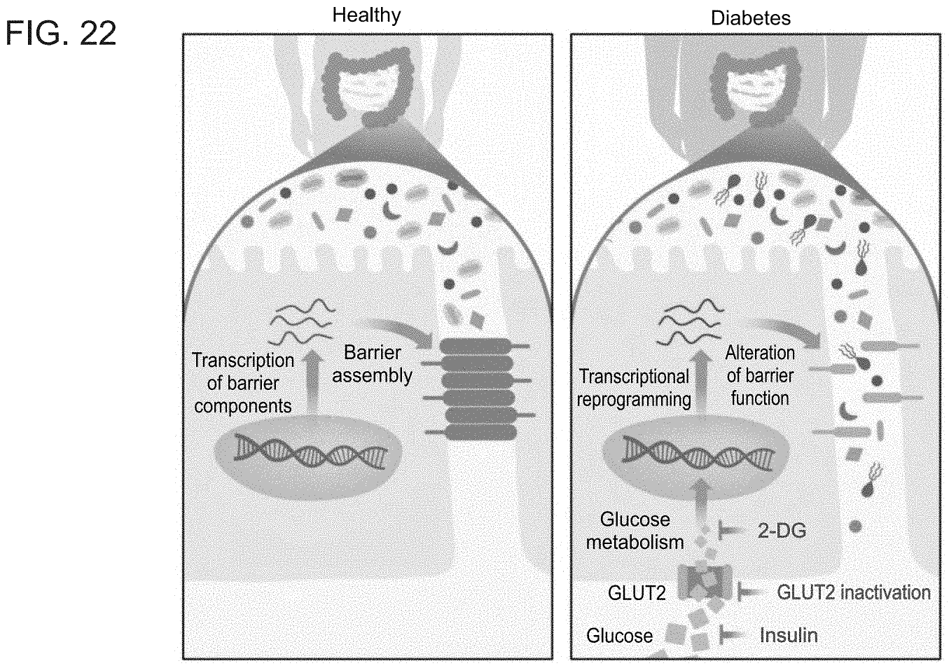

Using a novel ex-vivo screening platform, the present inventors have identified glucose (as well as additional agents) as an orchestrator of intestinal barrier function. Hyperglycemia markedly interfered with homeostatic epithelial integrity, leading to abnormal influx of immune-stimulatory microbial products and a propensity for systemic spread of enteric pathogens. The results indicate that hyperglycemia causes retrograde transport of glucose into intestinal epithelial cells via GLUT2, followed by alterations in intracellular glucose metabolism and transcriptional reprogramming (FIG. 22). One of the most strongly affected pathways by hyperglycemia in the present examples involves the N-glycosylation of proteins in the endoplasmic reticulum and Golgi apparatus, which has been implicated as a key regulator of a multitude of epithelial functions.

Collectively, the present findings provide a potential molecular explanation for altered barrier function in the context of the metabolic syndrome and the resultant enhanced mucosal infection noted in patients suffering from obesity and diabetes mellitus. Furthermore, the link highlighted by the present inventors between hyperglycemia and gut barrier alterations may provide a mechanistic basis for a variety of seemingly unrelated inflammatory manifestations, complications and associations of the metabolic syndrome (collectively termed ‘metaflammation’ or ‘para-inflammation’). Examples of these include adipose tissue inflammation driving exacerbated obesity and glucose intolerance, non-alcoholic fatty liver disease progressing to detrimental non-alcoholic steatohepatitis, inflammation contributing to atherosclerosis and associated cardiovascular disease and even recently suggested associations between the metabolic syndrome and neurodegeneration. Ultimately, the present results may present the starting point for harnessing glucose metabolism or other regulators of intestinal barrier integrity as potential therapeutic targets in the prevention and amelioration of enteric infection and gut-related systemic inflammation.

Thus, according to a first aspect of the present invention there is provided a method of identifying agents useful for treating a disease associated with intestinal barrier dysfunction of a subject, the method comprising:

(a) contacting tight junctions of epithelial cells with a disrupting agent that disrupts or destabilizes tight junctions of epithelial cells; and

(b) contacting the tight junctions of epithelial cells with a test agent; and

(c) analyzing the effect of the test agent on the tight junctions of epithelial cells,

wherein when the test agent prevents disruption of, or stabilizes the tight junctions, it is indicative of the test agent being useful for treating the disease associated with intestinal barrier dysfunction of the subject.

In one embodiment, the disease associated with intestinal barrier dysfunction is a disease of the gastrointestinal tract (GIT), examples of which include, but are not limited to inflammatory bowel disease, metabolic syndrome, gut infection, celiac disease, non-celiac gluten sensitivity, food allergy and gut autoinflammation.

In a particular embodiment, the disease is an inflammatory bowel disease.

Inflammatory bowel diseases (IBD) are severe gastrointestinal disorders characterized by intestinal inflammation and tissue remodeling, that increase in frequency and may prove disabling for patients. The major forms of IBD, ulcerative colitis (UC) and Crohn's disease are chronic, relapsing conditions that are clinically characterized by abdominal pain, diarrhea, rectal bleeding, and fever.

Another disease associated with intestinal barrier dysfunction includes nonalcoholic fatty liver disease (NAFLD).

Another disease associated with intestinal barrier dysfunction includes non-alcoholic steatohepatitis (NASH).

Additional diseases associated with intestinal barrier dysfunction include infectious diseases, more specifically enteric infectious diseases. Examples of bacteria that may bring about intestinal barrier dysfunction in a subject include Escherichia coli, Vibrio cholerae, and several species of Salmonella, Shigella, and anaerobic streptococci. The subject may be characterized by symptoms such as diarrhea, abdominal discomfort, nausea and vomiting.

Other diseases which are associated with intestinal barrier dysfunction include diseases associated with systemic inflammation. These include diseases such as cancer, aging and neurodegeneration.

Metabolic diseases are also associated with intestinal barrier dysfunction. Such diseases include, but are not limited to obesity, hyperglycemia, type II diabetes, insulin resistance, prediabetes and glucose intolerance.

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, pancreatic, skin, prostate, and ovarian.

The assay described in this aspect of the present invention is effected in-vitro or ex-vivo.

Examples of epithelial (or epithelial-like) cell lines which may be used in this assay include, but are not limited to CaCo-2 cells, DLD-1 cells, HT-29 cells, T-84 cells and LoVo cells.

The present invention also contemplates use of primary cells. The epithelial cells may be derived from a subject having a disease associated with intestinal barrier dysfunction.

The epithelial cells are cultured under conditions that promote tight junction formation between the cells.

The term “tight junction (TJ),” as used herein, describes the closely associated apical areas of two cells whose membranes join together by specialized transmembrane proteins forming a virtually impermeable barrier to fluid.

Preferably, the epithelial cells are cultured as a monolayer. Examples of media that can be used to culture the epithelial cells include, but are not limited to DMEM, EMEM and RPMI.

In one embodiment, the cells are cultured directly on a solid surface (e.g. plastic, glass etc.). Alternatively, the solid surface is coated with an adhesive matrix, such as an extracellular matrix protein. Examples of contemplated extracellular matrix proteins include, but are not limited to collagen (e.g. type I collagen), Matrigel™ or fibronectin.

Preferably, the cells are cultured for at least 12 hours, more preferably at least 24 hours so as to promote generation of coherent and uniform monolayers with highly organized tight junctions. When an extracellular matrix protein is used to coat the solid surface, the cells may be cultured such that stable focal adhesions are formed with the underlying ECM. The thickness of the underlying ECM coating may be adjusted for experiments aiming at the analysis of tight junctions and those used for testing focal adhesions.

The assay may be performed on a microtiter plate—e.g. 6, 12, 24, 48, 96, 384 or 1536 sample wells arranged in a 2:3 rectangular matrix.

Once the tight junctions are generated between the epithelial cells, a disrupting agent is used which is capable of disrupting or destabilizing the tight junctions.

As used herein, the term “disrupting agent” refers to an agent that is capable of disrupting or destabilizing tight junctions of epithelial cells as assayed by light microscopy compared to a control (i.e. absence of the agent) under identical conditions in less than 48 hours, more preferably less than 24 hours.

In one embodiment, the disrupting agent has a morphological effect similar to TNFα and LPS on epithelial cells when assayed by light microscopy. In another embodiment, the disrupting agent has a morphological effect similar to histamine and spermine on epithelial cells when assayed by light microscopy.

Examples of disruptors that can be used to disrupt/destabilize the tight junctions include those listed in Tables 5, 7 or 9, listed in the Examples section herein below.

Other disruptors that may be used include the bacterial surface antigen—LPS or the bacterial metabolite−histamine.

In another embodiment, the disruptor is comprised in a sample derived from the gastrointestinal tract of a subject having the disease associated with intestinal barrier dysfunction. In this way the assay may be used in a personalized fashion, tailoring the agent to the specific disease of the subject.

Thus, the present invention contemplates contacting tight junctions of epithelial cells with a sample derived from the GIT of the subject under conditions which are conducive to disruption or destabilization of tight junctions of epithelial cells.

The sample may be a fecal sample or an intestinal mucosal sample.

In some embodiments, the sample is a microbiota sample which is collected by any means that allows recovery of the microbes and without disturbing the relative amounts of microbes or components or products thereof of the microbiome. In some embodiments, the microbiota sample is a fecal sample. In other embodiments, the microbiota sample is retrieved directly from the gut—e.g. by endoscopy from the lower gastrointestinal (GI) tract or from the upper GI tract. The microbiota sample may be of the lumen of the GI tract or the mucosa of the GI tract.

According to one embodiment the microbiota sample (e.g. fecal sample) is frozen and/or lyophilized prior to analysis. According to another embodiment, the sample may be subjected to solid phase extraction methods.

During the assay, a test agent is also added to the cultured epithelial cells.

The test agent may be a pharmaceutical agent, a small molecule, a polypeptide, a polynucleotide, a bacteria, a bacterial metabolite, a carbohydrate, a lipid or a combination of the same.

“Small molecules” can be, for example, naturally occurring compounds (e.g., compounds derived from plant extracts, microbial broths, and the like) or synthetic organic or organometallic compounds having molecular weights of less than about 10,000 daltons, preferably less than about 5,000 daltons, and most preferably less than about 1,500 daltons.

In one embodiment, the test agent is one that downregulates an amount and/or activity of a component of a sample derived from the gastrointestinal tract (GIT) of a subject having the disease, the component being present in an amount in the sample which is up-regulated compared to the amount in a sample derived from the GIT of a healthy subject.

In still another embodiment, the test agent is one that upregulates an amount or activity of a component of a sample derived from the gastrointestinal tract (GIT) of a subject having the disease, the component being present in an amount in the sample which is down-regulated compared to the amount in a sample derived from the GIT of a healthy subject.

Samples derived from the GIT of subjects are described herein above. According to a particular embodiment, the sample is a microbiota sample derived from the GIT (e.g. fecal sample).

The present invention contemplates analyzing the samples and identifying components (e.g. bacteria, bacterial metabolites etc.) that are up- or down-regulated in the samples compared to samples derived from healthy subjects. Thus, for example, if a component is down-regulated in the subject having the disease, that agent (or an agent that increases its activity or amount) may be tested in the assay of the present invention to see if it prevents disruption of the tight junctions. If a component is up-regulated in the subject having the disease, an agent that decreases its activity or amount may be tested in the assay of the present invention to see if it prevents disruption of the tight junctions.

A component which is up-regulated in a sample of a diseased subject compared to in a sample of a healthy subject is one which is present in an amount which is at least 20%, 30%, 40%, 50%, 60%, 70%, 80%, 90% or even 100% higher than in the sample of the healthy subject.

A component which is down-regulated in a sample of a diseased subject compared to in a sample of a healthy subject is one which is present in an amount which is at least 20%, 30%, 40%, 50%, 60%, 70%, 80%, 90% or even 100% lower than in the sample of the healthy subject.

It will be appreciated that for comparison, the sample type of the healthy subject should be identical to the sample type of the diseased subject. Thus, for example if the sample type of the diseased subject is a fecal sample, then the sample type of the healthy subject should also be a fecal sample.

Quantifying Microbial Levels:

The abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, as further described herein below.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differ between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR) and then sequencing. In some embodiments, a level or set of levels of one or more DNA sequences is determined by amplifying DNA sequences using quantitative PCR. These and other basic DNA amplification procedures are well known to practitioners in the art and are described in Ausubel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation.

This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods.

According to one embodiment, the sequencing method allows for quantitating the amount of a microbe—e.g. by deep sequencing such as Illumina deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the level of microbes, the present invention also contemplates analyzing the level of microbial products.

Examples of microbial products include, but are not limited to mRNAs, polypeptides, carbohydrates and metabolites.

As used herein, a “metabolite” is an intermediate or product of metabolism. The term metabolite is generally restricted to small molecules and does not include polymeric compounds such as DNA or proteins. A metabolite may serve as a substrate for an enzyme of a metabolic pathway, an intermediate of such a pathway or the product obtained by the metabolic pathway.

According to a particular embodiment, the metabolite is one that alters the composition or function of the microbiome.

In preferred embodiments, metabolites include but are not limited to sugars, organic acids, amino acids, fatty acids, hormones, vitamins, oligopeptides (less than about 100 amino acids in length), as well as ionic fragments thereof. Cells can also be lysed in order to measure cellular products present within the cell. In particular, the metabolites are less than about 3000 Daltons in molecular weight, and more particularly from about 50 to about 3000 Daltons.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

Representative examples of metabolites that may be analyzed according to this aspect of the present invention include, but are not limited to bile acid components such as ursodeoxycholate, glycocholate, phenylacetate and heptanoate and flavonoids such as apigenin and naringenin.

In some embodiments, levels of metabolites are determined by mass spectrometry, as further described herein below. In some embodiments, levels of metabolites are determined by nuclear magnetic resonance spectroscopy, as further described herein below. In some embodiments, levels of metabolites are determined by enzyme-linked immunosorbent assay (ELISA). In some embodiments, levels of metabolites are determined by colorimetry. In some embodiments, levels of metabolites are determined by spectrophotometry, as further described herein below.

As mentioned, the test agent is contacted with the epithelial cells of the assay.

In one embodiment, the test agent is added concomitantly (i.e. essentially at the same time) with the disruptor agent.

In another embodiment, the test agent is added following addition of the disruptor agent (e.g. at least 6 hours following addition of the disruptor agent, at least 12 hours following addition of the disruptor agent, at least 24 hours following addition of the disruptor agent, at least 48 hours following addition of the disruptor agent). The test agent may be added once the disrupted or destabilized tight junctions are formed.

In yet another embodiment, the test agent is added prior to the addition of the disruptor agent (e.g. at least 6 hours prior to the addition of the disruptor agent, at least 12 hours prior to the addition of the disruptor agent, at least 24 hours prior to the addition of the disruptor agent, at least 48 hours prior to the addition of the disruptor agent. In this way, it may be able to know whether the test agent is capable of preventing formation of disrupted or destabilized tight junctions.

Following addition of the test agent and the disruptor agent, the tight junctions of the epithelial cells are analyzed. Preferably, the analyzing is carried out no more than 12 hours, 24 hours, 48 hours or three days following the addition of the test agent.

One method of analyzing the tight junctions is by measuring the structure of the disrupted or destabilized tight junctions—i.e. morphological analysis—this may be effected by immunohistochemical methods which involve detection of a substrate in situ in fixed cells by substrate specific antibodies. The substrate specific antibodies may be attached to detectable or reporter moieties. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

Examples of antibodies that can be used to analyze the structure of the tight junctions include those that bind to markers of tight junctions. Such markers include, but are not limited to cingulin, claudin-3, claudin-4, occluding and zonula occludens-1 (ZO-1).

Other examples of antibodies that can be used to analyze the structure of the tight junctions include those that bind to markers of focal adhesions. Such markers include, but are not limited to paxillin, zyxin, vincilin and tensin.

Various types of detectable or reporter moieties may be conjugated to the antibody of the invention. These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, U K. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody [e.g., horseradish peroxidase (HPR), beta-galactosidase, and alkaline phosphatase (A P)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule, which binds the affinity tag, can be fluorescently labeled or conjugated to enzyme as described above.

Various methods, widely practiced in the art, may be employed to attach a streptavidin or biotin molecule to the antibody. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson). Table 1 provides non-limiting examples of identifiable moieties which can be conjugated to the antibody of the invention.

TABLE 1

| | Amino Acid | Nucleic Acid |
|---|---|---|
| | sequence | sequence |
| | (GenBank | (GenBank |
| Identifiable Moiety | Accession No.) | Accession No.) |
| Green Fluorescent protein | AAL33912 | AF435427 |
| Alkaline phosphatase | AAK73766 | AY042185 |
| Peroxidase | CAA00083 | A00740 |
| Histidine tag | Amino acids | Nucleotides |
| | 264-269 of | 790-807 of |
| | GenBank | GenBank |
| | Accession No. | Accession No. |
| | AAK09208 | AF329457 |
| Myc tag | Amino acids | Nucleotides |
| | 273-283 of | 817-849 of |
| | GenBank | GenBank |
| | Accession No. | Accession No. |
| | AAK09208 | AF329457 |
| Biotin lygase tag | | |
| orange fluorescent protein | AAL33917 | AF435432 |
| Beta galactosidase | ACH42114 | EU626139 |
| Streptavidin | AAM49066 | AF283893 |

Other methods of detecting expression of tight junction markers and/or focal adhesion markers are listed herein below:

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to a particular embodiment, the analyzing is carried out quantitatively. Software may be used to quantitate the effect of the agent—e.g. based on advanced Watershed algorithms and/or basic MatLab tools.

Once an agent has been found that morphologically prevents disruption of tight junctions, it may be tested in other ways to corroborate its candidacy for treating the disease associated with intestinal barrier dysfunction. Thus, for example the agents may be tested functionally for preventing disruption of intestinal barrier dysfunction. Such tests include for example testing for trans-epithelial resistance using an Ussing chamber—e.g. as described in the Examples section herein below.

In vivo models may also be used. Exemplary in vivo animal models for testing agents for treating IBD are summarized in Hoffman et al., Pathobiology 2002-03; 70:121-130, and Kiesler et al, Cell Mol Gastroenterol; Hepatol 2015; 1:154-170 the contents of which are incorporated herein by reference.

It will be appreciated that the assay described herein may serve additional purposes—such as screening pharmaceutical agents for potential side effects on the intestinal barrier.

Thus, according to another aspect of the present invention there is provided a method of screening agents for potential side effect on the instestinal barrier, the method comprising:

(a) contacting tight junctions of epithelial cells with a disrupting agent that disrupts or destabilizes tight junctions of epithelial cells; and

(b) contacting the tight junctions of epithelial cells with a test agent; and

(c) analyzing the effect of the test agent on the tight junctions of epithelial cells, wherein when the test agent disrupts the tight junctions, it is indicative of the test agent has a side effect on the intestinal barrier (and therefore may be contraindicated).

As detailed herein above, the assay may be personalized using epithelial cells from a candidate subject. This may be particularly relevant in subjects who are known to have compromised epithelial intestinal barrier. When a test subject does not disrupt the tight junctions, it is indicative that the test agent is safe for treatment and may be provided to the subject.

The assay may also be used to identify agents that have a disrupting effect on the epithelial cells. Such agents may be used to increase the oral bioavailability of pharmaceutical agents, as further described herein below.

As mentioned, the assay of the present invention may comprise epithelial cells of the GIT of the subject and microbes derived from the GIT of the subject. Thus, the present inventors contemplate co-culture systems comprising cells of a tissue derived from the GIT of the subject and microbes derived from a microbiome of the GIT of the subject. The co-culture system may comprise media which allows the cells and the microbes to remain viable and optionally also propagate. Other co-culture systems are also contemplated comprising cells of a tissue derived from other organs and microbes derived from the microbiome of that organ.

Exemplary microbiomes contemplated by the present invention include, but are not limited to a skin microbiome, a gut microbiome, an intestinal microbiome, a mouth microbiome and a vaginal microbiome.

The co-culture system of the present invention may comprise more than one cell type derived from a tissue of the organ—for example two, three, four or more cell types. Additionally, or alternatively, the co-culture system may comprise more than one cell type derived from different tissues of the same organ.

Exemplary cells contemplated for use in the co-culture system include skin epithelial/endothelial cells, mouth epithelial/endothelial cells, vaginal epithelial/endothelial cells.

The present inventors have found a number of agents, which have been shown to prevent disruption of tight junctions and propose that such agents can be used to treat diseases associated with same.

Thus according to another aspect of the present invention there is provided a method of treating a disease associated with intestinal barrier dysfunction in a subject in need thereof, the method comprising administering to the subject a therapeutically effective amount of an agent which increases the amount and/or activity of at least one agent set forth in Table 2, thereby treating the disease associated with intestinal barrier dysfunction.

TABLE 2

| | |
|---|---|
| IL-21 | Interleukin-21 |
| CCL-3 | Chemokine ligand 3 |
| TIMP-2 | Tissue metalloprotease inhibitor 2 |
| tryptamine | Tryptophan Metabolism |
| L-homoserine | Glycine, Serine |
| Mitoxantrone Hydrochloride | DNA intercalating agent |
| Flavopiridol hydrochloride | CDK inhibitor |
| Dinaciclib (SCH727965) | CDK inhibitor |
| SNS-032 (BMS-387032) | CDK-2 inhibitor |
| AT7519 | multi-CDK inhibitor |
| Abemaciclib (LY2835219) | CDK4/6 inhibitor |
| CHIR-124 | Chk1 inhibitor |
| NH125 | eEF-2 kinase inhibitor |
| VX-702 | p38 MAPK inhibitor |
| CP 673451 | PDGFR inhibitor |
| PRT062607 (P505-15,) HCl | Syk inhibitor |
| GDC-0879 | Raf and ERK inhibitor |
| A-674563 | Akt1 inhibitor |
| GSK2126458 | PI3K inhibitor |
| RITA (NSC 652287) | MDM2 inhibitor |
| Acitretin | Retinoic acid derivate |
| Tazarotene (Avage) | Retinoic acid derivate |
| Bexarotene | Retinoic acid derivate |
| Tetrahydropapaverine hydrochloride | Phosphodiesterase (PDE) inhibitor |
| Alverine Citrate | Ca++ channel blocker |
| 5-hydroxymethyl tolterodine | muscarinic receptor antagonist |
| Lorcaserin HCl | 5-HT (Serotonin) Receptor agonist |
| Vortioxetine hydrobromide | 5-HT Receptor agonist/SSRI |
| L-Thyroxine | thyroid hormone |
| Carbimazole | thyroid peroxidase inhibitor |
| Tetrandrine (Fanchinine) | Ca++ channel blocker |
| Shikimic acid (Shikimate) | precursor of aromatic amino acids |
| Ketoconazole | antifungal drug |
| Enrofloxacin | bacterial topoisomerase inhibitor |
| Sesamin (Fagarol) | Food supplement |
| Vanillylacetone | Antioxidant |

In addition, the present inventors have found a number of agents that promote disruption of tight junction. Down-regulation of the amount and/or activity of such agents can be used to treat said diseases.

Thus, according to still another aspect of the present invention there is provided a method of treating a disease associated with intestinal barrier dysfunction in a subject in need thereof, the method comprising administering to the subject a therapeutically effective amount of an agent which decreases the amount and/or activity of at least one agent set forth in Table 3, thereby treating the disease associated with intestinal barrier dysfunction.

TABLE 3
disruptors

| | |
|---|---|
| IL-15 | Interleukin-15 |
| CCL-20 | Chemokine ligand 20 |
| CCL-23 | Chemokine ligand 23 |
| FGF1 | Fibroblast growth factor 1 (acidic) |
| FGF10 | Fibroblast growth factor 10 |
| BMP10 | Bone morphogenetic protein 10 |
| UTS2 | Urotensin 2 |
| UCN1 | Urocortin 1 |
| UCN3 | Urocortin 3 |
| EPO | erythropoietin |
| putrescine | Polyamine Metabolism |
| N-acetylproline | Urea cycle, arginine and proline |
| | metabolism |
| MLN8237 (Alisertib) | Aurora Kinase inhibitor |
| Danusertib (PHA-739358) | Aurora Kinase, Bcr-Abl, |
| | c-RET, FGFR inhibitor |
| Rigosertib (ON-01910) | PLK/Src inhibitor |
| KX2-391 | Src inhibitor |
| AT7867 | Akt inhibitor |
| GDC-0068 | Akt inhibitor |
| LY2109761 | TGFbeta inhibitor |
| PF-04691502 | PI3K/mTOR inhibitor |
| TG101348 (SAR302503) | JAK |
| | inhibitor |
| CEP33779 | JAK inhibitor |
| LY2784544 | JAK inhibitor |
| Baricitinib (LY3009104) | JAK |
| | inhibitor |
| NVP-AEW541 | IGF-1R inhibitor |
| NVP-ADW742 | IGF-1R inhibitor |
| PHA-848125 | CDK + Thropomyosin receptor |
| | kinase A inhibitor |
| CI994 (Tacedinaline) | HDAC inhibitor |
| GSK 269962 | ROCK inhibitor |
| RKI-1447 | ROCK inhibitor |
| Lexibulin (CYT997) | Mt depolymerization |
| NPI-2358 (Plinabulin) | Mt depolymerization |
| Epothilone A | Mt stabilization similar to taxanes |
| SCH 79797 | PARI antagonist |
| Gemcitabine | nucleoside analog |
| Puerarin (Kakonein) | 5-HT antagonist |
| Dioscin (Collettiside III) | Saponin |
| Indole-3-carbinol | food- |
| | supplement |
| Berberine Hydrochloride | plant |
| | alcaloid |

Subjects which can be treated according to any of the aspects of the present invention are typically mammalian—e.g. human.

Examples of contemplated diseases associated with intestinal barrier dysfunction which may be treated according to any of the aspects of the present invention are described herein above. According to a particular embodiment, the disease associated with intestinal barrier dysfunction is an acute disease or disorder. According to another embodiment, the disease associated with intestinal barrier dysfunction is a chronic disease or disorder

When the agent which appears in Table 3 is a polypeptide, the present invention contemplates downregulating expression thereof. It will be appreciated that downregulating activity of the polypeptides is also contemplated. Methods of downregulating expression of proteins are further described herein below.

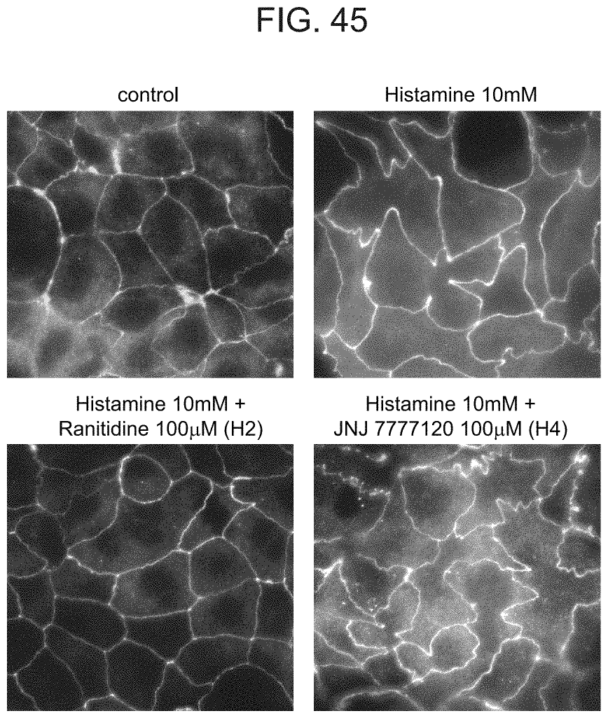

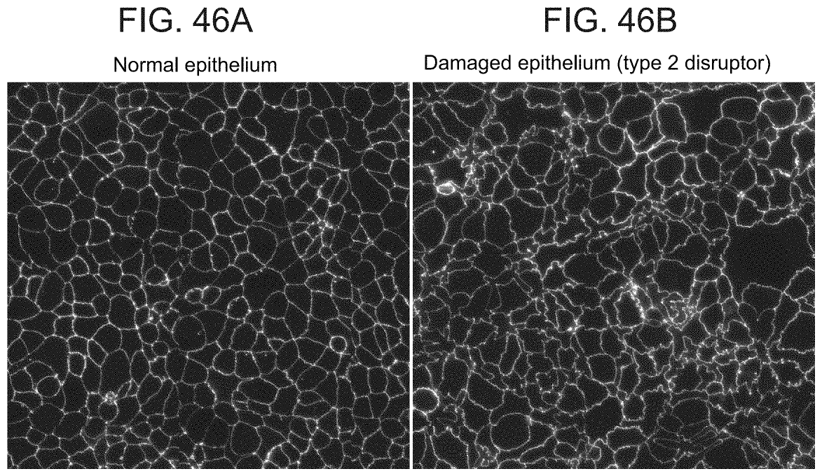

The present inventors have found that histamine receptor antagonists block the disruptive effects of histamine on the tight junctions of epithelial cells—see FIG. 45. Thus, the present inventors promote treating subjects with diseases associated with intestinal barrier dysfunction with histamine receptor antagonists. Examples of H2 receptor antagonists include, but are not limited to Cimetidine, Famotidine, Lafutidine, Nizatidine, Ranitidine, Roxatidine and Tiotidine.

The present inventors have also found that intracellular glucose promotes disruption of tight junctions and enhances intestinal barrier dysfunction.

Thus, according to yet another aspect of the present invention there is provided a method of treating a disease associated with intestinal barrier dysfunction in a subject in need thereof, the method comprising administering to the subject a therapeutically effective amount of an agent which downregulates the amount of glucose in intestinal cells, with the proviso that the disease is not Diabetes or obesity, thereby treating the disease associated with intestinal barrier dysfunction.

The agent of this aspect of the invention may block glucose entry into intestinal cells, and/or downregulate glucose levels within intestinal cells.

For this aspect of the present invention, the subject may or may not be suffering from a metabolic comorbidity such as obesity, hyperglycemia, type II diabetes, insulin resistance, coronary heart disease, glucose intolerance, cerebrovascular disease and/or high blood pressure.

Preferably, the agent downregulates the amount of intracellular glucose in intestinal cells to a greater extent than the agent downregulates the amount of glucose in non-intestinal cells.

According to one embodiment, the agent is an inhibitor of glucose metabolism.

In another embodiment, the agent is an inhibitor of a glycolytic enzyme, examples of which are summarized in Table 4 herein below.

TABLE 4

| Compound name | Target protein |
|---|---|
| 2-DG | Inhibits HK |
| 3-BP | Inhibits HK |
| Lonidamine | Inhibits mitochondrial HK2 |
| 3PO | Inhibits PFK2 |
| N4A, YZ9 | Inhibits PFK2 |
| PGMI-004A | Inhibits PGAM1 |
| MJE3 | Inhibits PGAM1 |
| TT-232 | Inhibits PKM2 |
| Shikonin/alkannin | Inhibits PKM2 |
| ML265 (TEPP-46) | Activates PKM2 |
| FX11 | Inhibits LDHA |
| Quinoline 3-sulfonamides | Inhibit LDHA |
| DCA | Inhibits PDK |
| 6-AN | Inhibits G6PD |
| Oxythiamine | Inhibits TKTL1 |

2-DG: 2-deoxyglucose;
3-BP: 3-bromopyruvate;
DCA: Dichloroacetate;
6-AN: 6-aminonicotinamide;
HK: Hexokinase;
PFK: Phosphofructokinase;
PGAM: Phosphoglycerate mutase;
PKM2: Pyruvate kinase M2;
LDH: Lactate dehydrogenase;
PDK: Pyruvate dehydrogenase kinase;
G6PD: Glucose-6-phosphate dehydrogenase;
TKTL1: Transketolase-like enzyme 1

According to one embodiment of this aspect of the present invention, the agent is an inhibitor of a glucose transporter.

As used herein, the term “glucose transporter” refers to a protein that transports compounds (whether glucose, glucose analogs, other sugars such as fructose or inositol, or non-sugars such as ascorbic acids) across a cell membrane and are members of the glucose transporter “family” based on structural similarity (e.g., homology to other glucose transport proteins). Glucose transporters also include transporter proteins that have a primary sugar substrate other than glucose. For example, the glucose transporter GLUTS is primarily a transporter of fructose, and is reported to transport glucose itself with low affinity. Similarly, the primary substrate for the glucose transporter HMIT is myo-inositol (a sugar alcohol). Examples of glucose transporter include, but are not limited to GLUT1-12, HMIT and SGLT1-6 transporters.

According to a particular embodiment, the glucose transporter is GLUT-2.

An example of a glucose transporter inhibitor (e.g. a GLUT-2 transporter) is a flavonoid, such as a flavonol (e.g. a quercetin selected from the group consisting of aglycone quercetin, quercetin glycoside, and isoquercetin).

Another example of an inhibitor of a glucose transporter contemplated by the present invention is a cell-permeable thiazolidinedione compound marketed by Calbiochem (catalogue number 400035).

According to a particular embodiment, the agent downregulates expression of the glucose transporter.

As used herein the phrase “downregulates expression” refers to downregulating the expression of a protein at the genomic (e.g. homologous recombination and site-specific endonucleases) and/or the transcript level using a variety of molecules which interfere with transcription and/or translation (e.g., RNA silencing agents).

For the same culture conditions the expression is generally expressed in comparison to the expression in a cell of the same species but not contacted with the agent or contacted with a vehicle control, also referred to as control.

Down-regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Down-regulation at the nucleic acid level is typically effected using a nucleic acid agent, having a nucleic acid backbone, DNA, RNA, mimetics thereof or a combination of same which hybridizes to the endogenous caspase-6 encoding sequence (DNA or RNA, depending on the particular agent) of the cell. The nucleic acid agent may be encoded from a DNA molecule or provided to the cell per se (i.e. the RNA molecule is delivered directly to the cell).

Following is a description of various exemplary methods used to downregulate expression of a gene of interest (e.g. glucose transporter) and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—

Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas System—

Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

In order to cut DNA at a specific site, Cas9 proteins require the presence of a gRNA and a protospacer adjacent motif (PAM), which immediately follows the gRNA target sequence in the targeted polynucleotide gene sequence. The PAM is located at the 3′ end of the gRNA target sequence but is not part of the gRNA. Different Cas proteins require a different PAM. Accordingly, selection of a specific polynucleotide gRNA target sequence (e.g., on the glucose transporter nucleic acid sequence) by a gRNA is generally based on the recombinant Cas protein used.

The gRNA comprises a “gRNA guide sequence” or “gRNA target sequence” which corresponds to the target sequence on the target polynucleotide gene sequence that is followed by a PAM sequence.

The gRNA may comprise a “G” at the 5′ end of the polynucleotide sequence. The presence of a “G” in 5′ is preferred when the gRNA is expressed under the control of the U6 promoter. The CRISPR/Cas9 system of the present invention may use gRNA of varying lengths. The gRNA may comprise at least a 10 nts, at least 11 nts, at least a 12 nts, at least a 13 nts, at least a 14 nts, at least a 15 nts, at least a 16 nts, at least a 17 nts, at least a 18 nts, at least a 19 nts, at least a 20 nts, at least a 21 nts, at least a 22 nts, at least a 23 nts, at least a 24 nts, at least a 25 nts, at least a 30 nts, or at least a 35 nts of the target glucose transporter DNA sequence which is followed by a PAM sequence. The “gRNA guide sequence” or “gRNA target sequence” may be at least 17 nucleotides (17, 18, 19, 20, 21, 22, 23), preferably between 17 and 30 nts long, more preferably between 18-22 nucleotides long. In an embodiment, gRNA guide sequence is between 10-40, 10-30, 12-30, 15-30, 18-30, or 10-22 nucleotides long.

Although a perfect match between the gRNA guide sequence and the DNA sequence on the targeted gene is preferred, a mismatch between a gRNA guide sequence and target sequence on the gene sequence of interest is also permitted as along as it still allows hybridization of the gRNA with the complementary strand of the gRNA target polynucleotide sequence on the targeted gene. A seed sequence of between 8-12 consecutive nucleotides in the gRNA, which perfectly matches a corresponding portion of the gRNA target sequence is preferred for proper recognition of the target sequence. The remainder of the guide sequence may comprise one or more mismatches. In general, gRNA activity is inversely correlated with the number of mismatches. Preferably, the gRNA of the present invention comprises 7 mismatches, 6 mismatches, 5 mismatches, 4 mismatches, 3 mismatches, more preferably 2 mismatches, or less, and even more preferably no mismatch, with the corresponding gRNA target gene sequence (less the PAM). Preferably, the gRNA nucleic acid sequence is at least 90%, 91%, 92%, 93%, 94%, 95%, 96%, 97%, 98% and 99% identical to the gRNA target polynucleotide sequence in the gene of interest (e.g., glucose transporter). Of course, the smaller the number of nucleotides in the gRNA guide sequence the smaller the number of mismatches tolerated. The binding affinity is thought to depend on the sum of matching gRNA-DNA combinations.

Any gRNA guide sequence can be selected in the target gene, as long as it allows introducing at the proper location, the patch/donor sequence of the present invention. Accordingly, the gRNA guide sequence or target sequence of the present invention may be in coding or non-coding regions of the glucose transporter gene (i.e., introns or exons).

The number of gRNAs administered to or expressed in a cell (or subject) or subject in accordance with the methods of the present invention may be at least 1 gRNA, at least 2 gRNAs, at least 3 gRNAs at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, at least 16 gRNAs, at least 17 gRNAs, or at least 18 gRNAs. The number of gRNAs administered to or expressed in a cell may be between at least 1 gRNA and at least 15 gRNAs, at least 1 gRNA to and least 10 gRNAs, at least 1 gRNA and at least 8 gRNAs, at least 1 gRNA and at least 6 gRNAs, at least 1 gRNA and at least 4 gRNAs, at least 1 gRNA to and least 3 gRNAs, at least 2 gRNA and at least 5 gRNAs, at least 2 gRNA and at least 3 gRNAs. Different or identical gRNAs may be used to cut the endogenous target gene of interest and liberate the donor/patch nucleic acid, when provided in a vector.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

The Cas protein that may be used in accordance with the present invention has a nuclease (or nickase) activity to introduce a double stranded break (DSB) (or two single stranded breaks (SSBs) in the case of a nickase) in cellular DNA when in the presence of appropriate gRNA(s).

In one embodiment, the Cas9 protein is a recombinant protein.

In another embodiment, the Cas9 protein is derived from a naturally occurring Cas9 which has nuclease activity and which function with the gRNAs of the present invention to introduce double stranded breaks in the targeted DNA.

In an embodiment, the Cas9 protein is a dCas9 protein (i.e., a mutated Cas9 protein devoid of nuclease activity) fused with a dimerization-dependent FokI nuclease domain. In another embodiment, the Cas protein is a Cas9 protein having a nickase activity.

Cas9 proteins are natural effector proteins produced by numerous species of bacteria including Streptococcus pyogene, Streptococcus thermophiles, Staphylococcus aureus, and Neisseria meningitides. Accordingly, in an embodiment, the Cas protein of the present invention is a Cas9 nuclease/nickase derived from Streptococcus pyogene, Streptococcus thermophiles, Staphylococcus aureus or Neisseria meningitides. In an embodiment, the Cas9 recombinant protein of the present invention is a human-codon optimized Cas9 derived from S. pyogenes (hSpCas9). In an embodiment, the Cas9 recombinant protein of the present invention is a human-codon optimized Cas9 derived from S. aureus (hSaCas9).

Non-limiting examples of viral vectors which can be used to express the Cas9 and/or gRNA include retrovirus, lentivirus, Herpes virus, adenovirus or adeno Associated Virus, as well known in the art. Herpesvirus, adenovirus, Adeno-Associated virus and lentivirus derived viral vectors have been shown to efficiently infect neuronal cells. Preferably, the viral vector is episomal and not cytotoxic to cells. In an embodiment, the viral vector is an AAV or a Herpes virus.

Downregulation of a glucose transporter can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

In one embodiment, the RNA silencing agents of the present invention (including gRNAs, which are further described herein above) are modified polynucleotides. Polynucleotides can be modified using various methods known in the art.

For example, the oligonucleotides or polynucleotides of the present invention may comprise heterocylic nucleosides consisting of purines and the pyrimidines bases, bonded in a 3′-to-5′ phosphodiester linkage.

Preferably used oligonucleotides or polynucleotides are those modified either in backbone, internucleoside linkages, or bases, as is broadly described hereinunder.

Specific examples of preferred oligonucleotides or polynucleotides useful according to this aspect of the present invention include oligonucleotides or polynucleotides containing modified backbones or non-natural internucleoside linkages. Oligonucleotides or polynucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Additionally, or alternatively the oligonucleotides/polynucleotide agents of the present invention may be phosphorothioated, 2-o-methyl protected and/or LNA modified. Oligonucleotides or polynucleotides of the present invention may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications.

The modified polynucleotide of the present invention may also be partially 2′-oxymethylated, or more preferably, is fully 2′-oxymethylated.

The RNA silencing agents (including gRNAs) designed according to the teachings of the present invention can be generated according to any oligonucleotide synthesis method known in the art, including both enzymatic syntheses or solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

According to an embodiment of the invention, the RNA silencing agent (including the gRNA described herein) is specific to the target RNA (e.g., glucose transporter) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi: 10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base-pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

According to another embodiment the RNA silencing agent may be a miRNA or a miRNA mimic.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above, that administration of a miRNA may be affected in a number of ways:
1. Administering the mature double stranded miRNA;
2. Administering an expression vector which encodes the mature miRNA;
3. Administering an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.
4. Administering an expression vector which encodes the pri-miRNA The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

Another agent capable of downregulating a polypeptide (e.g., glucose transporter) is a DNAzyme molecule capable of specifically cleaving an mRNA transcript or DNA sequence of the caspase. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNases see Khachigian, L M [Curr Opin Mol Ther 4:119-21 (2002)].

Downregulation of a polypeptide (e.g. glucose transporter) can also be effected by using an antisense polynucleotide capable of specifically hybridizing with an mRNA transcript encoding the glucose transporter.

Design of antisense molecules which can be used to efficiently downregulate a glucose transporter must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way, which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Another agent capable of downregulating a polypeptide (e.g. glucose transporter) is a ribozyme molecule capable of specifically cleaving an mRNA transcript encoding the glucose transporter. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of regulating the expression of a polypeptide (e.g. glucose transporter) gene in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the glucose transporter regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

As mentioned, disruptor agents (i.e. those which increase the permeability of the colonic epithelial barrier) may be used as agents which enhance the oral bioavailability of a therapeutically active agent.

According to a particular embodiment the disruptor agent may enhance absorption of a pharmaceutical agent.

Herein the term “therapeutically active agent” refers to the ingredient accountable for a therapeutic effect, as opposed, for example, to enhancement of absorption of the therapeutically active agent, which is effected by the disruptor agents.

Herein, the phrase “enhancing absorption” refers to causing an increase of at least 10% in levels (e.g., plasma levels) of absorbed agent.

In some embodiments of any one of the embodiments described herein, the therapeutically active agent has a molecular weight of at least 0.5 kDa. In some embodiments, the molecular weight is in a range of from 0.5 to 150 kDa. In some embodiments, the molecular weight is in a range of from 0.5 to 100 kDa. In some embodiments, the molecular weight is in a range of from 0.5 to 75 kDa. In some embodiments, the molecular weight is in a range of from 0.5 to 50 kDa. In some embodiments, the molecular weight is in a range of from 0.5 to 30 kDa. In some embodiments, the molecular weight is in a range of from 0.5 to 20 kDa. In some embodiments, the molecular weight is in a range of from 0.5 to 10 kDa. In some embodiments, the molecular weight is in a range of from 0.5 to 7.5 kDa. In some embodiments, the molecular weight is in a range of from 0.5 to 5 kDa.

In some embodiments of any one of the embodiments described herein, the therapeutically active agent has a molecular weight of at least 1 kDa. In some embodiments, the molecular weight is in a range of from 1 to 150 kDa. In some embodiments, the molecular weight is in a range of from 1 to 100 kDa. In some embodiments, the molecular weight is in a range of from 1 to 75 kDa. In some embodiments, the molecular weight is in a range of from 1 to 50 kDa. In some embodiments, the molecular weight is in a range of from 1 to 30 kDa. In some embodiments, the molecular weight is in a range of from 1 to 20 kDa. In some embodiments, the molecular weight is in a range of from 1 to 10 kDa. In some embodiments, the molecular weight is in a range of from 1 to 7.5 kDa. In some embodiments, the molecular weight is in a range of from 1 to 5 kDa.

In some embodiments of any one of the embodiments described herein, the therapeutically active agent has a molecular weight of at least 2 kDa. In some embodiments, the molecular weight is in a range of from 2 to 150 kDa. In some embodiments, the molecular weight is in a range of from 2 to 100 kDa. In some embodiments, the molecular weight is in a range of from 2 to 75 kDa. In some embodiments, the molecular weight is in a range of from 2 to 50 kDa. In some embodiments, the molecular weight is in a range of from 2 to 30 kDa. In some embodiments, the molecular weight is in a range of from 2 to 20 kDa. In some embodiments, the molecular weight is in a range of from 2 to 10 kDa. In some embodiments, the molecular weight is in a range of from 2 to 7.5 kDa. In some embodiments, the molecular weight is in a range of from 2 to 5 kDa.

In some embodiments of any one of the embodiments described herein, the therapeutically active agent has a molecular weight of at least 3 kDa. In some embodiments, the molecular weight is in a range of from 3 to 150 kDa. In some embodiments, the molecular weight is in a range of from 3 to 100 kDa. In some embodiments, the molecular weight is in a range of from 3 to 75 kDa. In some embodiments, the molecular weight is in a range of from 3 to 50 kDa. In some embodiments, the molecular weight is in a range of from 3 to 30 kDa. In some embodiments, the molecular weight is in a range of from 3 to 20 kDa. In some embodiments, the molecular weight is in a range of from 3 to 10 kDa. In some embodiments, the molecular weight is in a range of from 3 to 7.5 kDa. In some embodiments, the molecular weight is in a range of from 3 to 5 kDa.

In some embodiments of any one of the embodiments described herein, the therapeutically active agent has a molecular weight of at least 4 kDa. In some embodiments, the molecular weight is in a range of from 4 to 150 kDa. In some embodiments, the molecular weight is in a range of from 4 to 100 kDa. In some embodiments, the molecular weight is in a range of from 4 to 75 kDa. In some embodiments, the molecular weight is in a range of from 4 to 50 kDa. In some embodiments, the molecular weight is in a range of from 4 to 30 kDa. In some embodiments, the molecular weight is in a range of from 4 to 20 kDa. In some embodiments, the molecular weight is in a range of from 4 to 10 kDa. In some embodiments, the molecular weight is in a range of from 4 to 7.5 kDa. In some embodiments, the molecular weight is in a range of from 4 to 5 kDa.

In some embodiments of any one of the embodiments described herein, the therapeutically active agent has a molecular weight of at least 5 kDa. In some embodiments, the molecular weight is in a range of from 5 to 150 kDa. In some embodiments, the molecular weight is in a range of from 5 to 100 kDa. In some embodiments, the molecular weight is in a range of from 5 to 75 kDa. In some embodiments, the molecular weight is in a range of from 5 to 50 kDa. In some embodiments, the molecular weight is in a range of from 5 to 30 kDa. In some embodiments, the molecular weight is in a range of from 5 to 20 kDa. In some embodiments, the molecular weight is in a range of from 5 to 10 kDa. In some embodiments, the molecular weight is in a range of from 5 to 7.5 kDa.

In some embodiments of any one of the embodiments described herein, the therapeutically active agent has a molecular weight of at least 10 kDa. In some embodiments, the molecular weight is in a range of from 10 to 150 kDa. In some embodiments, the molecular weight is in a range of from 10 to 100 kDa. In some embodiments, the molecular weight is in a range of from 10 to 75 kDa. In some embodiments, the molecular weight is in a range of from 10 to 50 kDa. In some embodiments, the molecular weight is in a range of from 10 to 30 kDa. In some embodiments, the molecular weight is in a range of from 10 to 20 kDa.

In some embodiments of any one of the embodiments described herein, the therapeutically active agent has a molecular weight of at least 20 kDa. In some embodiments, the molecular weight is in a range of from 20 to 150 kDa. In some embodiments, the molecular weight is in a range of from 20 to 100 kDa. In some embodiments, the molecular weight is in a range of from 20 to 75 kDa. In some embodiments, the molecular weight is in a range of from 20 to 50 kDa. In some embodiments, the molecular weight is in a range of from 20 to 30 kDa.

In some embodiments of any one of the embodiments described herein, the therapeutically active agent has a molecular weight of at least 50 kDa. In some embodiments, the molecular weight is in a range of from 50 to 150 kDa. In some embodiments, the molecular weight is in a range of from 50 to 100 kDa. In some embodiments, the molecular weight is in a range of from 50 to 75 kDa.

Without being bound by any particular theory, it is believed that agents having a relatively high molecular weight (e.g., at least 0.5 kDa, at least 1 kDa, at least 2 kDa, at least 3 kDa, at least 4 kDa) tend to be less efficiently absorbed upon oral administration than relatively small molecules (e.g., molecules having a molecular weight of less than 0.5 kDa, or less than 1 kDa) and therefore, their absorption is particularly susceptible to enhancement by the disrupting agent.

In some embodiments of any one of the embodiments described herein, the therapeutically active agent is a hormone and/or cytokine (e.g., a hormone).

In some embodiments of any one of the embodiments described herein, the therapeutically active agent is a polypeptide.

Without being bound by any particular theory, it is believed that agents which are polypeptides tend to be poorly absorbed upon oral administration, for example, due to their polarity and/or relatively large molecular weight; and therefore, their absorption is particularly susceptible to enhancement by the disruptor agent.

In some embodiments, the polypeptide is a polypeptide hormone and/or cytokine, or a fragment thereof (e.g., a fragment exhibiting an activity of the hormone and/or cytokine), or a homolog of a polypeptide hormone and/or cytokine or fragment thereof.

Examples of polypeptides which may be utilized (per se or as fragments thereof and/or homologs thereof) as therapeutically active agents according to embodiments of the invention include, without limitation, insulin, a glucagon, a parathyroid hormone, an interferon, a growth hormone, an erythropoietin, a calcitonin, an omentin, a motilin, a leptin, a peptide YY, a GLP-1 (glucagon-like peptide-1), a GLP-2 (glucagon-like peptide-2), granulocyte-colony stimulating factor (G-CSF), an antibody (e.g., monoclonal antibody), an interleukin, an erythropoietin, a vasopressin, a vasoactive intestinal peptide, a pituitary adenylate cyclase-activating peptide (PACAP), a blood clotting factor, an endomorphin (e.g., endomorphin-1, endomorphin-2), a TNF inhibitor (e.g., infliximab, adalimumab, certolizumab, golimumab, etanercept), disitertide, octreotide (a somatotropin analog), davunetide, icatibant, glucocerebrosidase, a gonadotropin releasing hormone (GnRH), acyline (a GnRH antagonist), and a GLP-1 agonist such as exendin-4 (including exenatide and lixisenatide). Examples of growth hormones, include, without limitation, somatotropin (growth hormone 1), growth hormone 2, and growth factors (e.g., insulin-like growth factor 1 (IGF-1), fibroblast growth factor (FGF), ciliary neurotrophic factor).

Insulin, glucagon, parathyroid hormone, erythropoietin, calcitonin, motilin, leptin, peptide YY, GLP-1 (including derivatives thereof such as liraglutide, taspoglutide, albiglutide and dulaglutide), GLP-2, GnRH (including derivatives thereof such as leuprorelin, buserelin, histrelin, goserelin, deslorelin, nafarelin and triptorelin), vasopres sin (including derivatives thereof such as desmopressin), vasoactive intestinal peptide (including aviptadil), pituitary adenylate cyclase-activating peptide (PACAP), growth hormones (including axokine, a homolog of a fragment of ciliary neurotrophic factor) and G-CSF are non-limiting examples of polypeptide hormones.

Interferons, interleukins, erythropoietin and analogs thereof (e.g., darbepoetin), omentin and G-CSF are non-limiting examples of polypeptide cytokines.

It has been reported that therapeutically active agents which exhibit more than one of the following criteria tend to be poorly absorbed upon oral administration (when administered alone), a phenomenon referred to in the art as “Lipinski's rule of 5”:
(i) a total number of nitrogen-hydrogen bonds and oxygen hydrogen bonds (which are typically hydrogen bond donors) which is more than 5;
(ii) a total number of nitrogen and oxygen atoms (which are typically hydrogen bond acceptors) which is more than 5;
(iii) an octanol-water partition coefficient (log P) which is greater than 5; and/or
(iv) a molecular weight of at least 500 Da (0.5 kDa).

The abovementioned criteria (i) and (ii) are associated with hydrogen bonding and hydrophilicity; whereas criteria (iii) is associated with lipophilicity.

As described herein, therapeutically active agents poorly absorbed upon oral administration when administered alone are particularly suitable for being included in compositions described herein, in order to enhance their absorption.

In some embodiments of any one of the embodiments described herein, the therapeutically active agent meets at least one of the abovementioned criteria (i), (ii), (iii) and (iv). In some embodiments, the therapeutically active agent meets at least two of the abovementioned criteria (i), (ii), (iii) and (iv). In some embodiments, the therapeutically active agent meets at least three of the abovementioned criteria (i), (ii), (iii) and (iv). In some embodiments, the therapeutically active agent meets all four of the abovementioned criteria (i), (ii), (iii) and (iv).

In some embodiments of any one of the embodiments described herein, the therapeutically active agent has a molecular weight of at least 0.5 kDa, in accordance with any one of the embodiments described herein relating to a molecular weight of at least 0.5 kDa, and further meets at least one of the abovementioned criteria (i), (ii) and (iii). In some such embodiments, the therapeutically active agent meets at least two of the abovementioned criteria (i), (ii) and (iii).

Dihydroergotamine and fondaparinux are non-limiting examples of non-peptidic agents having a molecular weight of at least 0.5 kDa, which are poorly absorbed upon oral administration.

In some embodiments of any one of the embodiments described herein, the therapeutically active agent has a molecular weight of less than 0.5 kDa, and meets at least one of the abovementioned criteria (i), (ii) and (iii). In some such embodiments, the therapeutically active agent meets at least two of the abovementioned criteria (i), (ii) and (iii). In some such embodiments, the therapeutically active agent meets all three of the abovementioned criteria (i), (ii) and (iii).

In addition, ionic molecules tend to be poorly absorbed upon oral administration, generally due to a considerably reduced ability to cross lipid membranes. Whether a molecule is ionic or non-ionic often depends on pH, which varies according to location in the gastrointestinal tract. In general, it is believed that the more a therapeutically active agent is in ionic form in the gastrointestinal tract, the more likely it is to be poorly absorbed upon oral administration.

In some embodiments of any one of the embodiments described herein, the therapeutically active agent is ionic in an aqueous solution at a pH of 7.0.

In some embodiments of any one of the embodiments described herein, the therapeutically active agent is ionic in an aqueous solution at a pH of 6.0.

In some embodiments of any one of the embodiments described herein, the therapeutically active agent is ionic in an aqueous solution at a pH of 5.0.

In some embodiments of any one of the embodiments described herein, the therapeutically active agent is ionic in an aqueous solution at a pH of 4.0.

In some embodiments of any one of the embodiments described herein, the therapeutically active agent is ionic in an aqueous solution at a pH of 3.0.

In some embodiments of any one of the embodiments described herein, the therapeutically active agent is ionic in an aqueous solution at a pH of 2.0.

In some embodiments of any one of the embodiments described herein, the therapeutically active agent is ionic in an aqueous solution at a pH of 1.0.

Examples of such agents include, without limitation, compounds comprising at least one basic group (e.g., amine group) which is positively charged at a pH of 7.0 (or less).

Herein, a compound is considered “ionic” when it comprises at least one functional group which is charged in at least 50% of the molecules in a population of molecules of the compound under designated conditions (e.g., in an aqueous solution at a designated pH value or range of pH values). The skilled person will be readily capable of determining whether a functional group is charged in at least 50% of the molecules, for example, by determining a pKa value associated with the functional group. An ionic compound, as defined herein, may optionally have a net negative charge, optionally a net positive charge, and optionally an equal number of negatively charged functional groups and positively functional groups, resulting in no net charge.

In some embodiments of any one of the embodiments described herein, the therapeutically active agent is ionic in an aqueous solution at all pH values within a range of from 5.0 to 7.0. In some embodiments, the therapeutically active agent is ionic in an aqueous solution at all pH values within a range of from 5.0 to 8.0. In some embodiments, the therapeutically active agent is ionic in an aqueous solution at all pH values within a range of from 4.0 to 9.0. In some embodiments, the therapeutically active agent is ionic in an aqueous solution at all pH values within a range of from 3.0 to 10.0. In some embodiments, the therapeutically active agent is ionic in an aqueous solution at all pH values within a range of from 2.0 to 11.0.

In some embodiments of any one of the embodiments described herein, the therapeutically active agent is ionic at a pH value and/or range according to any one of the abovementioned embodiments, and further has a molecular weight of at least 0.5 kDa, in accordance with any one of the embodiments described herein relating to a molecular weight of at least 0.5 kDa. In some embodiments of any one of the embodiments described herein, the therapeutically active agent is ionic at a pH value and/or range according to any one of the abovementioned embodiments, and further has a molecular weight of less than 0.5 kDa.

Examples of ionic therapeutically active agents which tend to have a molecular weight of less than 0.5 kDa, and which tend to exhibit poor absorption upon oral administration, include, without limitation, bisphosphonates (e.g., for use in treating osteoporosis and related conditions) such as alendronate, clodronate, etidronate, ibandronate, neridronate, olpadronate, pamidronate, risedronate, tiludronate and zoledronate; and cromolyn (e.g., cromolyn sodium).

In some embodiments of any one of the embodiments described herein, the therapeutically active agent is a Class III agent according to the Biopharmaceutics Classification System (BCS), as provided by the U.S. FDA, that is, the therapeutically active agent is characterized by low permeability and high solubility.

In the context of the BCS, the phrase “low permeability” refers herein and in the art to absorption of less than 90% of a given agent upon oral administration in humans (in the absence of the disruptor agent), as determined by mass-balance determination and/or in comparison to an intravenous dose.

In some embodiments, absorption of a Class III therapeutically active agent is less than 50% upon oral administration (in the absence of the disruptor agent). In some embodiments, absorption is less than 20% upon oral administration (in the absence of the disruptor agent). In some embodiments, absorption is less than 10% upon oral administration (in the absence of the disruptor agent). In some embodiments, absorption is less than 5% upon oral administration (in the absence of the disruptor agent). In some embodiments, absorption is less than 2% upon oral administration (in the absence of the disruptor agent). In some embodiments, absorption is less than 1% upon oral administration (in the absence of the disruptor agent).

In the context of the BCS, the phrase “high solubility” refers herein and in the art to an amount of therapeutically active agent in an administered dose being soluble in 250 ml or less of water over a pH range of 1 to 7.5.

Exemplary disruptor agents that may be used for enhancing the bioavailability of therapeutically active agents include those set forth in Table 3, herein above. Additional disruptor agents include, but are not limited to Nocodazole and Vincristine, both of which are microtubule depolarizing agents, SCH 79797, Lenalidomide, Gemcitabine and Domperidone (Motilium).

The therapeutically active agent and the disruptor agent may be provided to the subject in a single formulation (i.e. co-formulated) or may be provided in separate formulations.

Preferably, the disruptor agent and the therapeutically active agents are administered orally as further described herein below.

The agents described in any of the aspects of the present invention may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially trans nasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

The agents described herein are administered such that they are capable of having a therapeutic effect in the intestine of the subject.

In one embodiment, the administration is such that the agents reach the intestine of the subject, but are not absorbed across the intestinal wall into the blood stream.

According to another embodiment, the agents are administered systemically, e.g. intravenously.

According to still another embodiment, the agents are administered intranasally.

According to yet another embodiment, the agents are administered orally.

Alternately, one may administer the pharmaceutical composition in a local rather than systemic manner, for example, via injection of the pharmaceutical composition.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions that can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The agents of the present invention may be comprised in particles (e.g. exosomes, microvesicles, nanvesicles, membrane particles, membrane vesicles, ectosomes and exovesicles). In other embodiments, the agents of the present invention may be comprised in synthetic particles (e.g. liposomes). The particles may be administered in any of the above mentioned ways including for example intranasal administration.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (down regulator of intracellular glucose) effective to prevent, alleviate or ameliorate symptoms of a disorder (e.g., IBD) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer, as further detailed below. Such information can be used to more accurately determine useful doses in humans.

Toxicity and therapeutic efficacy of the active ingredients described herein can be determined by standard pharmaceutical procedures in vitro, in cell cultures or experimental animals, as further detailed below. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to ensure blood or tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

According to a particular embodiment, the agent is administered for no more than 3 days, no more than 7 days, no more than two weeks, or no more than one month.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Hyperglycemia Drives Intestinal Barrier Dysfunction and Risk for Enteric Infection

Materials and Methods

Mice:

Wild-type C57Bl/6 mice were allowed to acclimatize to the animal facility environment for 2 weeks before used for experimentation. Germ-free C57Bl/6 mice were born in the Weizmann Institute germ-free facility and routinely monitored for sterility. The following genetically modified mice were used: B6.BKS(D)-Lepr^{db}/J, B6.Cg-Lep^{ob}/J, B6.Cg-Tg(Vil1-cre)1000Gum/J, B6.Cg-Tg(Alb-cre)21Mgn/J, B6.Cg-Tg(Nes-cre)1Kln/J, C57BL/6-Ins2^{Akita}/J, Sim1-Cre, SF1-Cre, ChAT-Cre, POMC-Cre, AgRP-Cre, LepR-flox mice and Villin-Cre:GLUT2^{fl/fl} mice on a C57BL/6 background.

In each experiment, all mice were littermates born and raised in the same vivarium. In all experiments, age- and gender-matched mice were used. All mice were maintained on a strict 12-h light-dark cycle (lights turned on at 6 am and turned off at 6 pm) and were housed in cages containing a maximum of five animals.

Before dietary interventions, mice were randomized to ensure that no incidental pre-diet differences in body weight existed between the different groups. Mice were exposed to high-fat diet for 12 weeks (Open Source Diets D12492) or normal chow diet (Teklad 2018) as indicated. In paired-feeding experiments, the food consumed by the wild-type group was weighted daily, and the same amount of food was made available to the db/db group for the subsequent day.

To induce hyperglycemia, streptozotocin (STZ, Sigma-Aldrich) was diluted freshly in PBS and injected i.p. at 100 mg/kg at two consecutive days. Control mice were injected with PBS. Serum glucose measurements were performed using glucose strips (Perrigo). Hyperglycemic mice were used for further experimentation 2-3 weeks after STZ injection). For continuous insulin administration, ALZET osmotic pumps were used. Pumps were implanted subcutaneously in the back of the mice for 4 weeks. Insulin was delivered at 0.25 U/day. 2-deoxyglucose (2-DG) was injected i.p. twice daily at 5 mg per injection as previously described (47). Injections were performed for 10 consecutive days. Leptin antagonist was administered daily by intraperitoneal injection of 25 mg/kg as previously described (18).

Fresh stool samples from mice were collected in tubes, immediately frozen in liquid nitrogen upon collection, and stored at −80° C. until DNA isolation. For antibiotic treatment, mice were given a combination of vancomycin (1 g/l), ampicillin (1 g/l), kanamycin (1 g/l), and metronidazole (1 g/l) in their drinking water (48). All antibiotics were obtained from Sigma Aldrich. For fecal transplantation experiments, microbiota samples were collected and homogenized under anaerobic conditions. Homogenized samples were filtered through a 70 μm mesh and administered to germ-free recipients by oral gavage of 200 μl.

Citrobacter rodentium Infection:

Mice were infected by oral gavage with 200 μl of an overnight culture of LB containing approximately 1×10⁹ colony-forming units (CFU) of a kanamycin-resistant, luciferase-expressing derivative of C. rodentium DBS100 (ICC180) as previously described (16). For in vivo bioluminescent imaging, mice were anesthetized with Ketamine/Xylazine. Bioluminescence was quantified using an IVIS2000 instrument and Living Image software (Perkin Elmer). For ex vivo luminescence analysis, colons were resected, extensively washed from all fecal matter and immediately imaged. For CFU counts, tissues were collected, weighed and homogenized in 1 ml of sterile PBS. Tissue homogenates were serially diluted in PBS and plated on to LB kanamycin plates, incubated overnight at 37° C., and bacterial colonies were enumerated the following day, normalizing them to the tissue weight.

Salmonella Typhimurium Infection:

For oral infections, mice were pretreated with 50 mg/ml streptomycin one day prior to oral gavage of 10⁸ CFU of SPI-II-deficient Salmonella Typhimurium (49). For systemic infections, mice were injected 10⁴ CFU of Salmonella Typhimurium by intraperitoneal injection as previously described (50). One week after infection, colonization was assessed by bacterial plating on LB streptomycin plates, incubated overnight at 37° C., and bacterial colonies were enumerated the following day, normalizing them to the tissue weight.

Quantification of Microbial Products at Systemic Sites:

The following PRR reporter cell lines were obtained from Invivogen (HEK-Blue TLR and NLR reporter cell lines): TLR2, TLR3, TLR4, TLR5, TLR7, TLR9, NOD1, NOD2. Extracts from spleen, liver, and serum were homogenized and added to reporter cell lines incubated with HEK-Blue detection medium (Invivogen) according to the manufacturer's instructions.

16S qPCR for Quantification of Bacterial DNA

DNA was extracted from samples using MoBio PowerSoil kit. DNA concentration was calculated using a standard curve of known DNA concentrations from E. coli K12. 16S qPCR using primers identifying different regions of the V6 16S gene was performed using Kappa SYBR fast mix. Absolute number of bacteria in the samples was then approximated as DNA amount in a sample/DNA molecule mass of bacteria. The same protocol was used to monitor sterility in germ-free mice.

Measuring Colonic Epithelial Barrier Permeability by FITC-Dextran:

On the day of the assay, 4 kDa fluorescein isothiocyanate (FITC)-dextran was dissolved in phosphate buffered saline (PBS) to a concentration of 80 mg/ml. Mice were fasted for 4 hours prior to gavage with 150 μl dextran. Mice were anesthetized 3 hours following gavage and blood was collected, centrifuged at 1,000×g for 12 min at 4° C. Serum was collected and fluorescence was quantified at an excitation wavelength of 485 nm and emission wavelength of 535 nm.

Purification of Intestinal Epithelial Cells for RNA Isolation:

Intestinal tissue was excised from mice, thoroughly rinsed with ice-cold PBS to clean the tissue from fecal matter, and opened longitudinally. The tissue was then cut into pieces of 1 cm length and incubated in HBSS containing 2 mM EDTA and 1 mM DTT at 37° C. for 30 mins while shaking at 130 rpm. Epithelial cells were collected, filtered, centrifuged, and subsequently stained with anti-EpCAM and anti-CD45 antibodies (Biolegend) for 30 mins on ice. Cells were then washed, resuspended and sorted into lysis/binding buffer (Life Technologies) using a FACS-FUSION cell sorter (BD).

RNA Isolation and RNA-Sequencing:

10⁴ cells from each population were sorted into 80 μl of lysis/binding buffer (Life Technologies). mRNA was captured with 12 μl of Dynabeads oligo(dT) (Life Technologies), washed, and eluted at 70° C. with 10 μl of 10 mM Tris-Cl (pH 7.5). A derivation of MARS-seq was used as described (51). An average of 1 million reads was sequenced per library (Illumina NextSeq). The reads were aligned to the mouse reference genome (NCBI 37, mm9) using TopHat v2.0.10 (52) with default parameters. Expression levels were calculated and normalized using ESAT software (garberlabdotumassmeddotedu/software/esat). Duplicate reads were filtered if they aligned to the same base and had identical UMIs. Expression levels were calculated and normalized for each sample to the total number of reads using HOMER software (hhomerdotsalkdotedu) with the command “analyzeRepeats.pl rna mm9-d [sample files]-count 3utr-condenseGenes” (53). KEGG analysis was done using DAVID (54).

Taxonomic Microbiota Analysis:

Frozen fecal samples were processed for DNA isolation using the MoBio PowerSoil kit according to the manufacturer's instructions. For the 16S rRNA gene PCR amplification, 1 ng of the purified fecal DNA was used for PCR amplification. Amplicons spanning the variable region V3/4 of the 16S rRNA gene were generated by using primers: Fwd 5′-GTGCCAGCMGCCGCGGTAA-3′ (SEQ ID NO: 1), Rev 5′-GGACTACHVGGGTWTCTAAT-3′ (SEQ ID NO: 2). The reactions were subsequently pooled and cleaned (PCR clean kit, Promega), and the PCR products were then sequenced on an Illumina MiSeq with 500 bp paired-end reads. The reads were then processed using the QIIME analysis pipeline as described (55, 56). In brief, fasta quality files and a mapping file indicating the barcode sequence corresponding to each sample were used as inputs, reads were split by samples according to the barcode, taxonomical classification was performed using the RDP-classifier, and an OTU table was created. Closed-reference OTU mapping was employed using the Greengenes database. Rarefaction was used to exclude samples with insufficient count of reads per sample. Sequences sharing 97% nucleotide sequence identity in the 16S region were binned into operational taxonomic units (97% ID OTUs). For beta-diversity, unweighted UniFrac measurements were plotted according to the two principal coordinates based on 10,000 reads per sample. For microbial distance measurements, unweighted UniFrac distances were compared.

Immunofluorescence Staining:

Colon samples were extensively washed and fixed and 4% paraformaldehyde. Samples were washed, paraffin-embedded and sectioned. Paraffin sections were de-paraffinized and antigen-retrieved in 10 mM sodium citrate, pH 6. Samples were incubated in PBS containing 20% (v/v) normal horse serum and 0.2% (v/v) Triton X-100 for 1 h; and then incubated over-night with rabbit anti-ZO-1 (Invitrogen 40-2200) primary antibody or rabbit anti E-cadherin (Cell Signaling 24E10). Sections were washed and incubated for 1 hour with alexa 488-conjugated donkey anti Rabbit antibody. Alternatively, sections were stained with Ki67 to indicate proliferating cells.

Flow Cytometry:

Colonic samples were extensively washed from fecal matter following by 2 mM EDTA dissociation at 37° C. for 30 mins. Following extensive shaking, the epithelial fraction was used for analysis of EpCAM⁺CD45⁻ cells. Colons were then digested using DNAaseI and Collagenase for lamina propria analysis. The isolated cells were washed with cold PBS and resuspended in PBS containing 1% BSA for direct cell surface staining. Single-cell suspensions were stained with antibodies for 30 min on ice against CD3, B220, CD11b, CD45, and CD127. Stained cells were analyzed on a BD-LSRFortessa cytometer and were analyzed with FlowJo software.

For viability measurements, isolated intestinal epithelial cells were stained with EpCAM, CD45 and propidium iodide. Cells were analyzed on a BD-LSRFortessa cytometer. All antibodies were obtained from Biolegend.

Ussing Chamber:

Epithelial resistance was measured using an Ussing chamber system (Warner Instruments P2300) according to the manufacturer's instructions. In brief, EasyMount chambers were calibrated, colonic tissue was excised from mice and immediately mounted, and voltage clamp recordings were performed. Tissues were maintained at 37° C. in physiological salt solution throughout the duration of the recording.

In Vitro Barrier Assessment:

Caco-2 cells were purchased from ATCC. Cells were routinely grown in regular tissue culture medium (DMEM, 10% FCS, penicillin/streptomycin, GlutaMAX, 1 g/l glucose). For experiments, cells were seeded onto 96-well glass-bottom plates coated with fibronectin 25 μg/ml 70 000 cells/well and allowed to grow for 48 hours to form confluent monolayer before treatment with indicated concentration of glucose for indicated time period. For immunofluorescent staining, cells were permeabilized with 0.5% Triton ×100 in 3% paraformaldehyde for 3 minutes and fixed with 3% paraformaldehyde for additional 30 minutes. Tight junctions labeling was performed with primary anti-ZO-1 mouse monoclonal antibodies (BD Transduction Laboratories) 1.25 μg/ml and secondary Alexa Fluor 488 goat anti-mouse antibodies (Invitrogen) 10 μg/ml. Cells were imaged using DeltaVision wide field fluorescent microscope (GE Healthcare) equipped with 40×UPlanFLN objective (Olympus). Cell images were segmented using Morphological Segmentation ImageJ plug-in and junction tortuosity was calculated as a ratio between junction length and Euclidian distance between its ends.

Human Cohort:

27 healthy volunteers were recruited for blood tests. The following variables were measured from each participant: height, waist circumference, weight, systolic and diastolic blood pressure, heart rate, and hip circumference. Obtained sera were subjected to PRR reporter detection assays (see above). Samples were additionally tested for the following parameters:

HbA1c, red blood cells, mean platelet volume, eosinophils ALT/GPT, lymphocytes, hematocrit, MCHC, hemoglobin, sodium, Red cell distribution width, creatinine, CRP, cholesterol, white blood cells, TSH, phosphorus, monocytes, MCH, HDL cholesterol, platelets, albumin, potassium, AST/GOT, neutrophils, MCV, chloride, basophils, and calcium. Pearson's correlation was used to compute correlations between different parameters.

sCD14 measurements:

Concentrations of sCD14 in the serum were measured using ELISA according to the manufacturer's instructions (DY383, R&D Systems). Briefly, plates were coated overnight with capture antibody, incubated with supernatant or serum, washed and incubated with anti-sCD14-biotin antibody and HRP-Avidin before quantification.

Statistics:

Data are expressed as mean±SEM. Comparisons between two groups were performed using Mann-Whitney U-test. ANOVA with Tukey's post-hoc test was used for comparison between multiple groups. K-means clustering based on Pearson's correlation was used to categorize elements in heatmaps. Survival was assessed by Mantel-Cox (log-rank) test. Linear regression was used to assess correlations between two data sets. P-values <0.05 were considered significant. * p<0.05; ** p<0.01; *** p<0.001; **** p<0.0001.

Results

Obesity is Associated with, but not Required for Intestinal Barrier Dysfunction

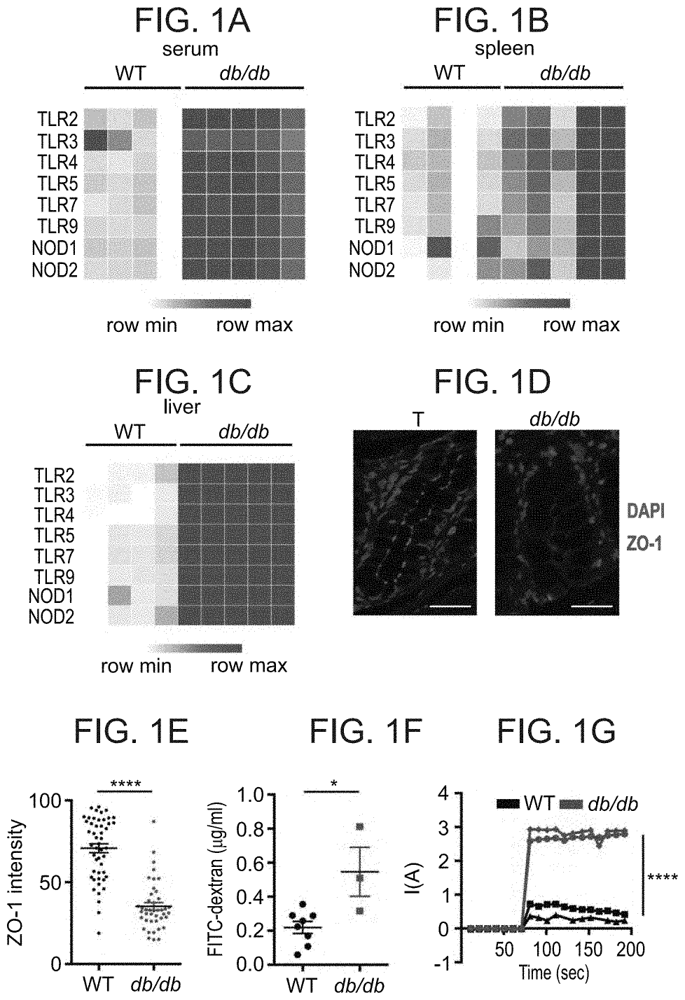

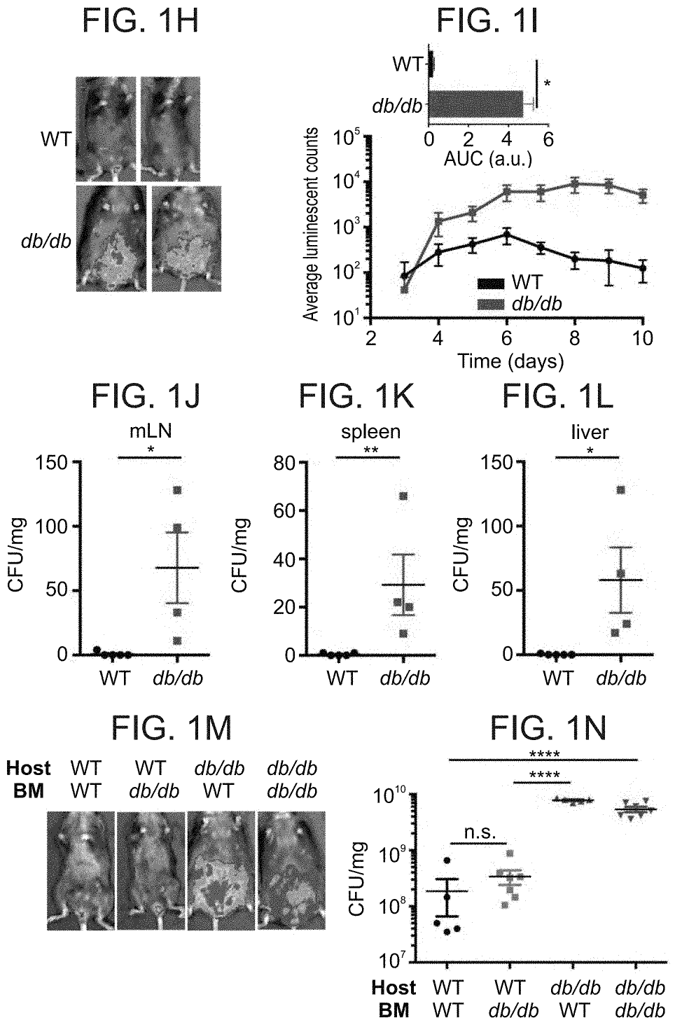

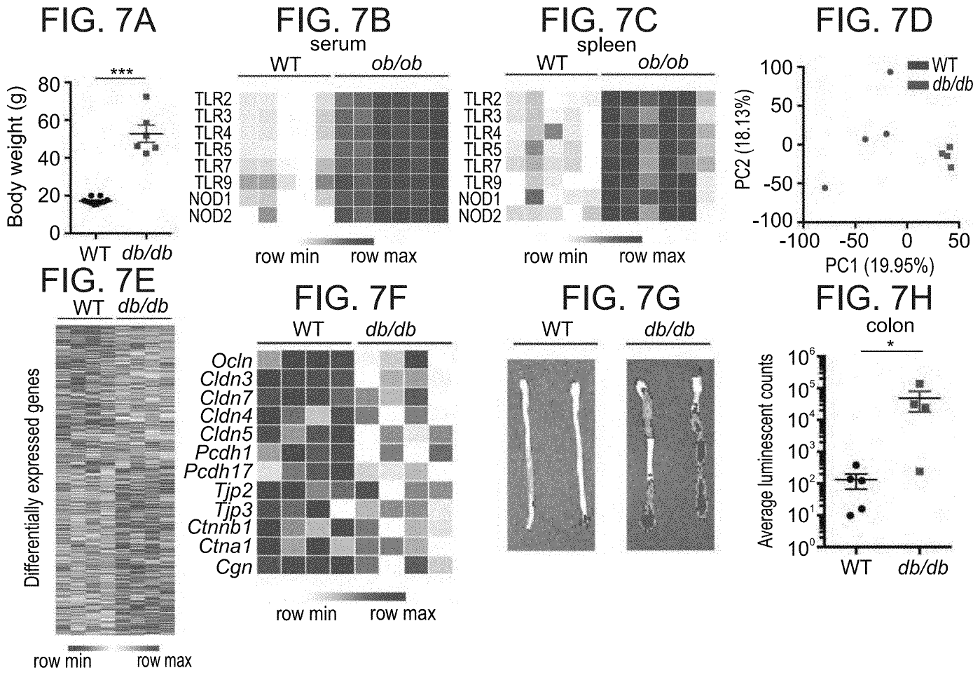

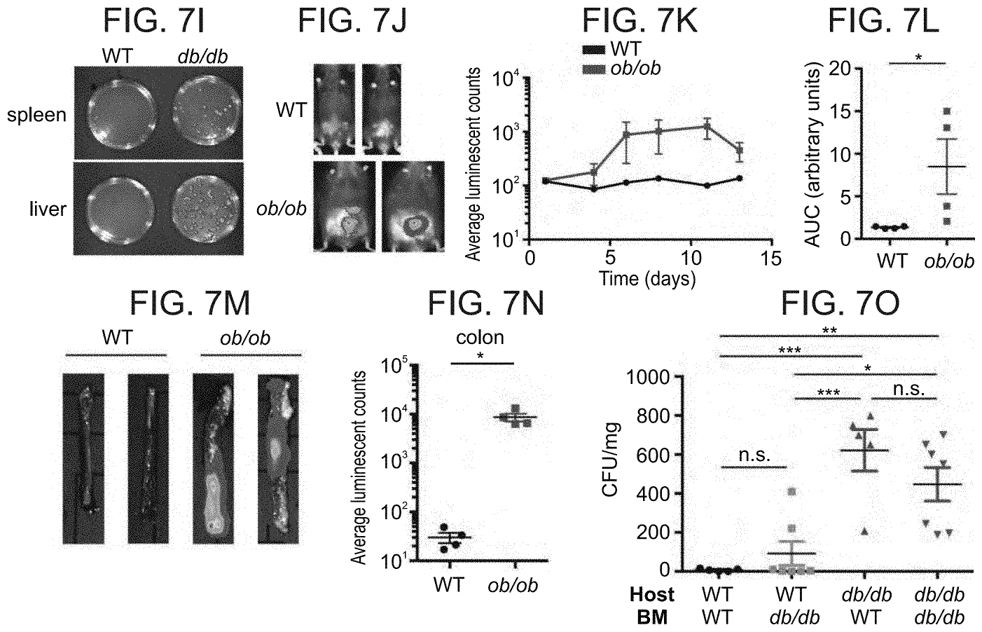

Adipokine leptin, a major orchestrator of mammalian satiety was analyzed to see whether it acted as an obesity-associated regulator of barrier integrity. Leptin deficiency and resistance to leptin signaling are strongly associated with morbid obesity in mice and humans, and both leptin deficiency and resistance were previously suggested to contribute to intestinal barrier dysfunction and susceptibility to enteric infection (10-13). In this example, a mouse model was used featuring genetic dysfunction of the leptin receptor (LepR), leading to hyperphagia and morbid obesity (db/db, FIG. 7A). Indeed, elevated levels of microbial pattern recognition receptor (PRR) ligands were detected at multiple systemic sites in leptin-unresponsive db/db mice (FIGS. 1A-C), indicative of enhanced influx of gut commensal-derived products. A similar phenomenon was observed in leptin-deficient mice (ob/ob, FIGS. 7B and 7C). To gain insight into the molecular signatures accompanying barrier dysfunction under aberrant leptin signaling, RNA-sequencing of colonic tissue, (obtained from db/db mice and their wild-type (WT) littermates under steady-state conditions) was performed. Leptin unresponsiveness was associated with global alterations of transcription (FIG. 7D), with several hundred genes featuring differential expression between both groups (FIG. 7E). Among the genes whose expression was most strongly abrogated in obese mice were members of the tight and adherence junction structures (FIG. 7F), protein complexes that inhibit para-cellular flux of intestinal molecules into the lamina propria (14). Consequently, tight junction integrity was compromised in db/db mice (FIGS. 1D and E), leading to enhanced influx of luminal molecules and electrical current measured across the epithelial layer (FIGS. 1F and G).

To determine the consequences of barrier dysfunction in leptin-resistant mice, the murine Citrobacter rodentium model was used simulating human enteropathogenic Escherichia coli infection (15). A bioluminescent variant of C. rodentium allowed for the noninvasive tracking of infection in vivo (16). In WT mice, C. rodentium caused a self-limiting, mainly gut-contained infection (FIG. 1H-L). In contrast, db/db mice did not clear the pathogen from their intestine (FIGS. 1H and I), in line with previous reports (12). Importantly, db/db mice also showed a significantly enhanced bacterial attachment to the intestinal wall (FIGS. 7G and H), and featured C. rodentium colonization at systemic sites (FIGS. 1J-L, and FIG. 71). Similar susceptibility to C. rodentium was noted for leptin-deficient ob/ob mice (FIGS. 7J-N).

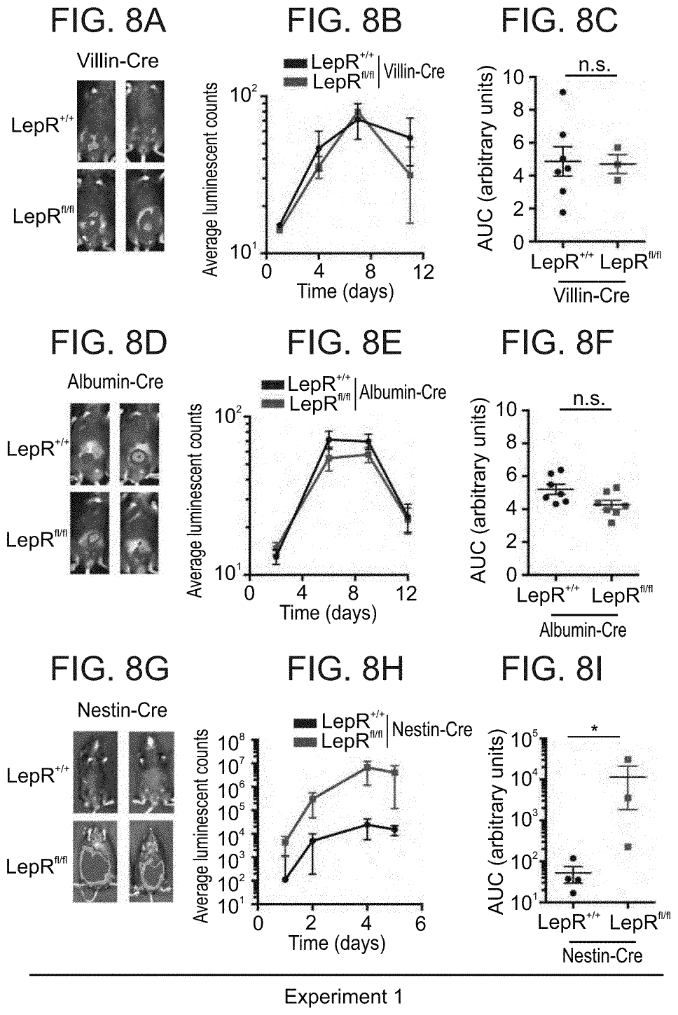

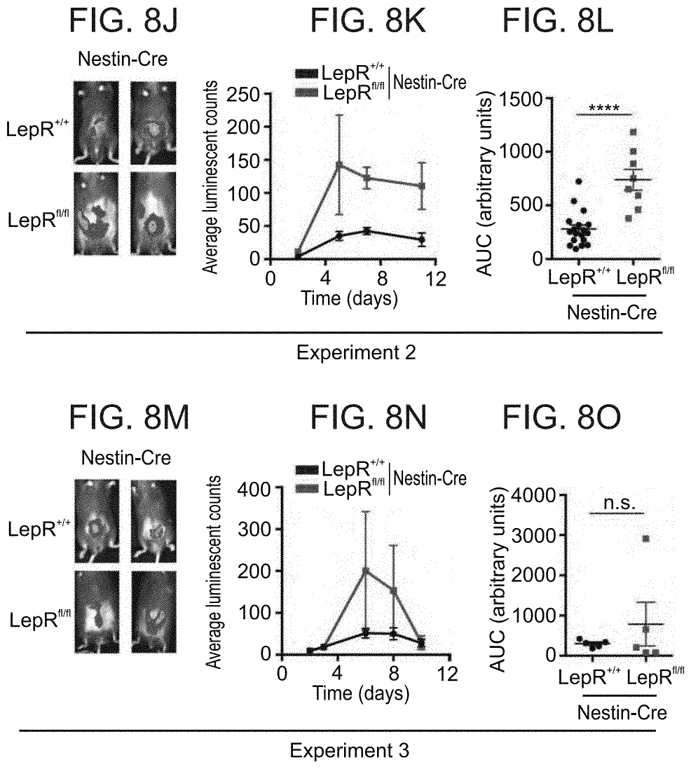

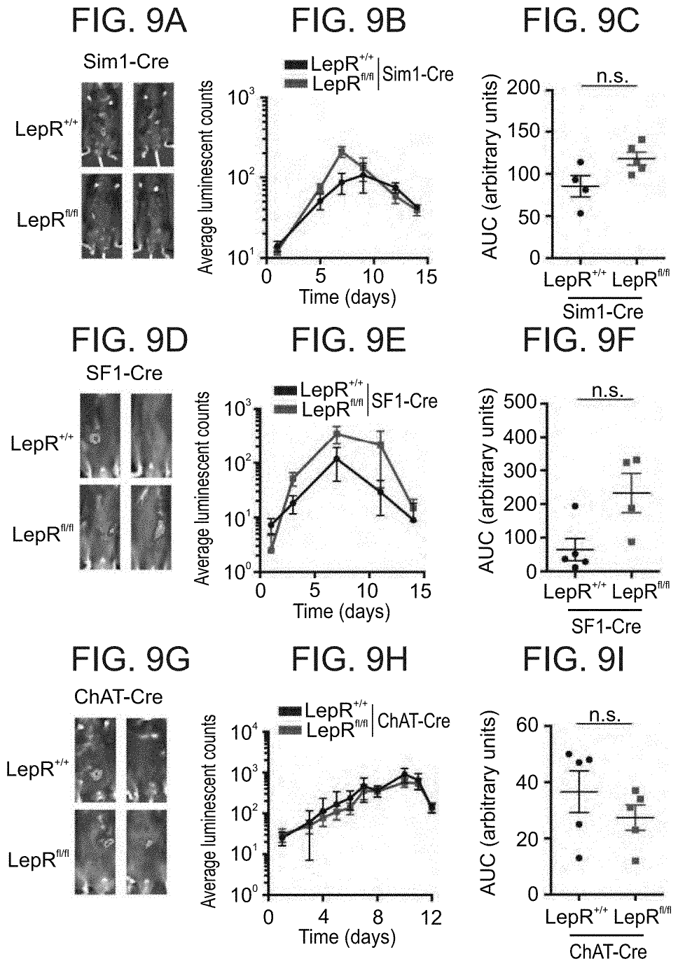

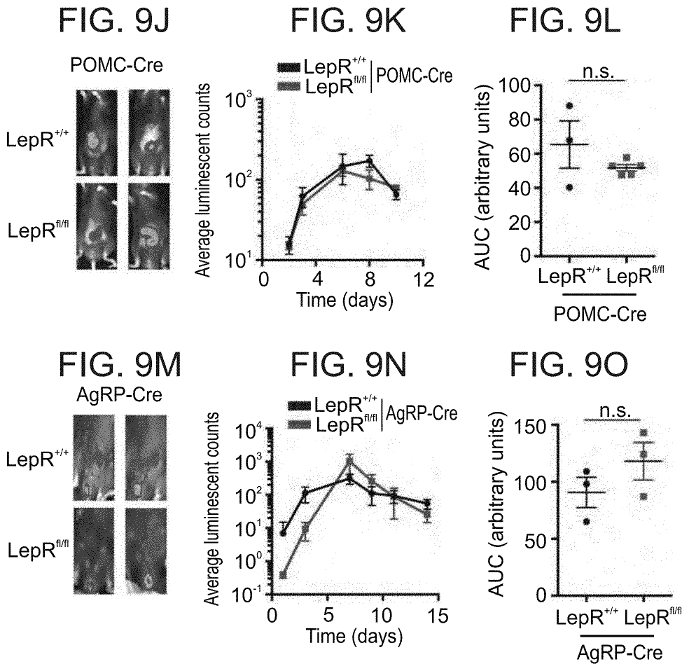

To understand which cell type was responsible for LepR-mediated protection from enteric infection, bone marrow chimeras were generated, in which WT and db/db mice were used as either recipients or donors of bone marrow transplanted into lethally irradiated mice. Exacerbated infection and systemic spread of C. rodentium was observed whenever the bone marrow recipient was LepR-deficient, regardless of the source of bone marrow (FIGS. 1M and N, and FIG. 70), indicating that the non-hematopoietic compartment mediated resistance against infection. LepR expression on non-hematopoietic cells has been reported in multiple tissues, including the gut, liver, and most prominently the nervous system (17). Mice lacking LepR in intestinal epithelial cells (Villin-Cre:LepR^{fl/fl}) or hepatocytes (Albumin-Cre:LepR^{fl/fl}) did not show any signs of enhanced susceptibility to C. rodentium infection (FIGS. 8A-F), while mice with LepR deficiency specifically in the nervous system (Nestin-Cre:LepR^{fl/fl}) featured an exacerbated (FIGS. 8G-I), yet highly variable (FIGS. 8J-0) bacterial growth. To further explore the possibility of neuronal leptin signaling driving barrier dysfunction and risk of infection, mice were generated with a specific deletion of LepR in the paraventricular hypothalamus (Sim1-Cre:LepR^{fl/fl}), the ventromedial hypothalamus (SF1-Cre:LepR^{fl/fl}), in cholinergic neurons (ChAT-Cre:LepR^{fl/fl}), and in the arcuate nucleus of the hypothalamus (POMC-Cre:LepR^{fl/fl} and AgRP-Cre:LepR^{fl/fl}). They were then infected with C. rodentium. However, none of these mice showed enhanced susceptibility to pathogenic invasion when compared to littermate controls (FIGS. 9A-O). Collectively, these results suggested that leptin deficiency per se might not provide a sufficient explanation to barrier dysfunction and enhanced risk of enteric infection.

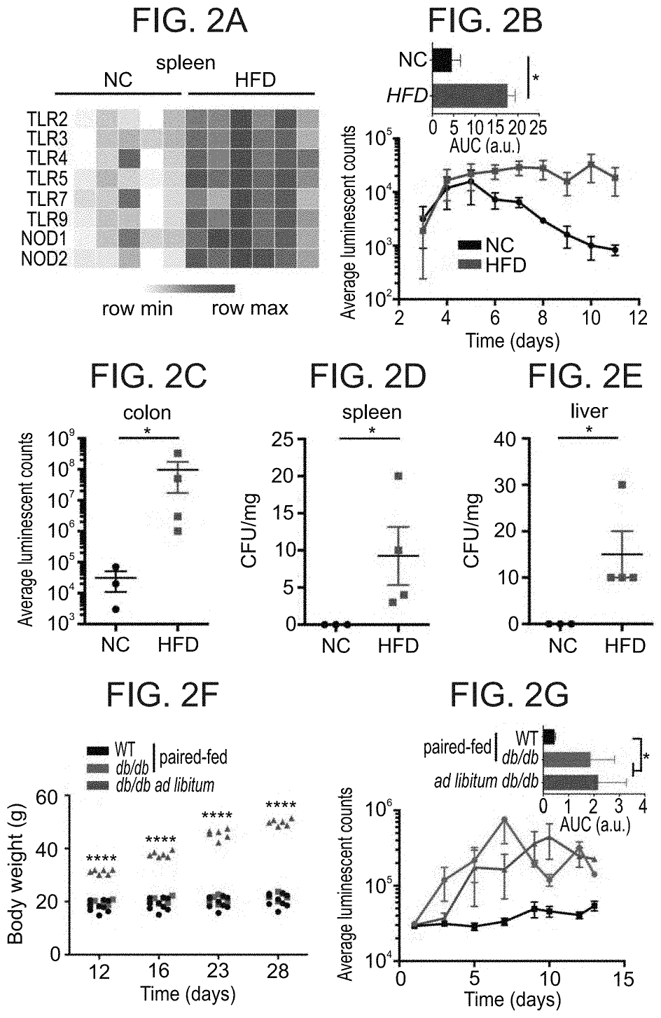

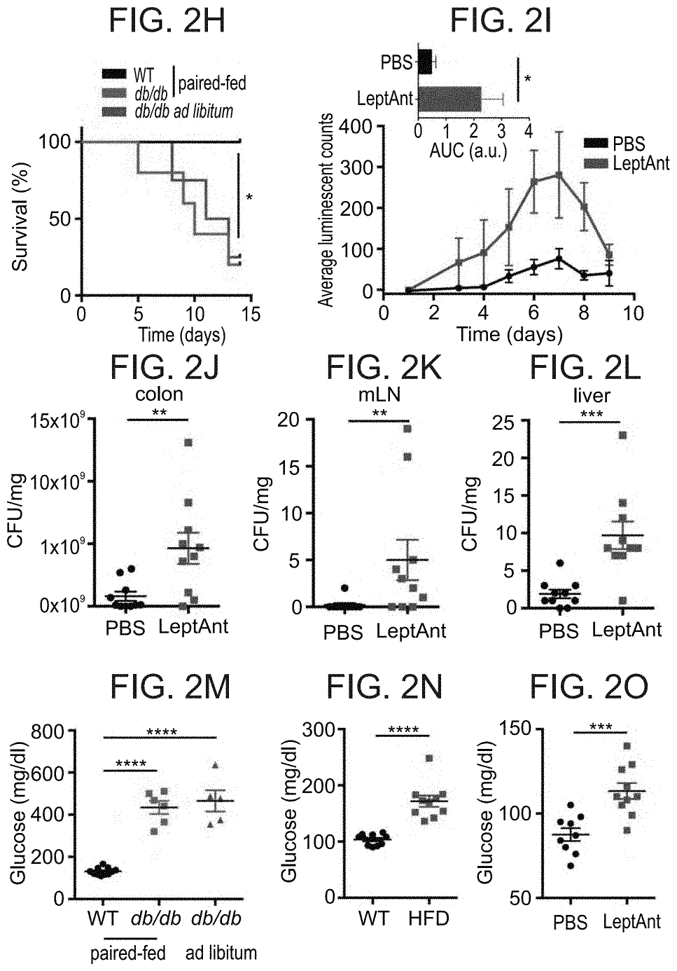

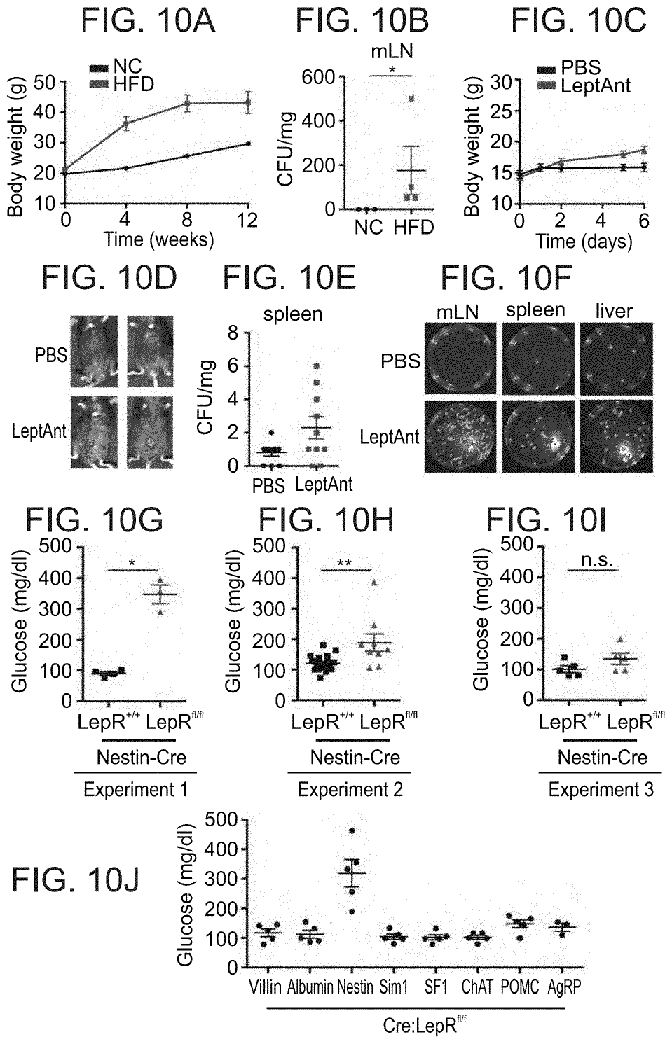

A feature common to all leptin- and LepR-deficient mice exhibiting an impaired barrier function and enhanced C. rodentium dissemination in the present examples (db/db, ob/ob and Nestin-Cre:LepR^{fl/fl}) was their tendency to develop obesity. The present inventors therefore hypothesized that an obesity-related factor distinct from leptin signaling may predispose these mice to impaired barrier function and exacerbated intestinal infection. Thus, to complement the above genetic models of obesity, WT mice were fed a high-fat diet (HFD) to induce weight gain (FIG. 10A). Similarly to obese leptin- and LepR-deficient mice, HFD-fed obese mice showed elevated steady-state systemic PRR ligand influx (FIG. 2A) as well as exacerbated C. rodentium infection and systemic dissemination (FIGS. 2B-E, and FIG. 10B). To further test whether obesity is the major driver for barrier dysfunction and impaired C. rodentium containment in LepR-deficient mice, paired-feeding experiments were performed, in which the food access for db/db mice was restricted to the amount consumed by their WT littermates, thereby equalizing body weight between both groups (FIG. 2F). Surprisingly, even after weight reduction to control levels, lean db/db mice were still unable to cope with C. rodentium infection (FIGS. 2G and 2H), ruling out that obesity per se was directly driving barrier dysfunction and risk for enteric infection in these mice. The lack of a direct causal relationship between obesity and barrier dysfunction was further supported by experiments using a chemical inhibitor of leptin signaling (18), which rendered WT mice susceptible to exacerbated infection and systemic bacterial spread even prior to the onset of marked obesity (FIGS. 2I-L, and FIGS. 10C-F). Together, these data indicated that neither leptin signaling nor obesity per se sufficiently explain the severity of barrier dysfunction and systemic enteric infection in mice with the metabolic syndrome.

In search of a unifying explanation for the above results in multiple mouse models of genetic and acquired obesity and leptin deficiency, the present inventors investigated other common features of the metabolic syndrome that could potentially contribute to barrier dysfunction. One such manifestation of the metabolic syndrome, typically accompanying obesity and potentially contributing to barrier dysfunction is glucose intolerance and resultant hyperglycemia. Interestingly, all mice featuring marked susceptibility C. rodentium infection, including obese db/db, pair-fed lean db/db mice, Nestin-Cre:LepR^{fl/fl}mice, mice fed a HFD, and mice treated with leptin antagonist showed elevated blood glucose levels (FIGS. 2M-O, and FIGS. 10G and 10H). In contrast, all mouse groups and models that did not develop enhanced C. rodentium susceptibility (Villin-Cre:LepR^{fl/fl}, Albumin-Cre:LepR^{fl/fl}, Sim1-Cre:LepR^{fl/fl}, SF1-Cre:LepR^{fl/fl}, ChAT-Cre:LepR^{fl/fl}, POMC-Cre:LepR^{fl/fl}and AgRP-Cre:LepR^{fl/fl}, as well as those Nestin-Cre:LepR^{fl/fl}mice that did not feature a tendency for severe infection) collectively showed normoglycemic levels (FIGS. 10I and 10J). Together, these results suggested that hyperglycemia, rather than obesity or alterations in leptin signaling, may predispose to barrier dysfunction leading to enhanced enteric infection in the setup of the metabolic syndrome in mice.

Hyperglycemia Drives Intestinal Barrier Disruption

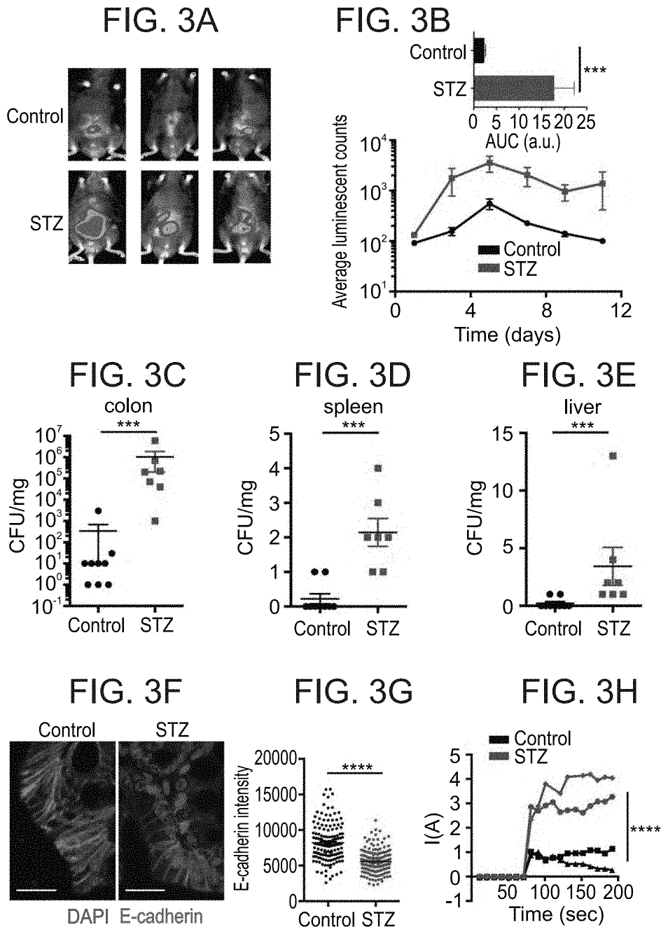

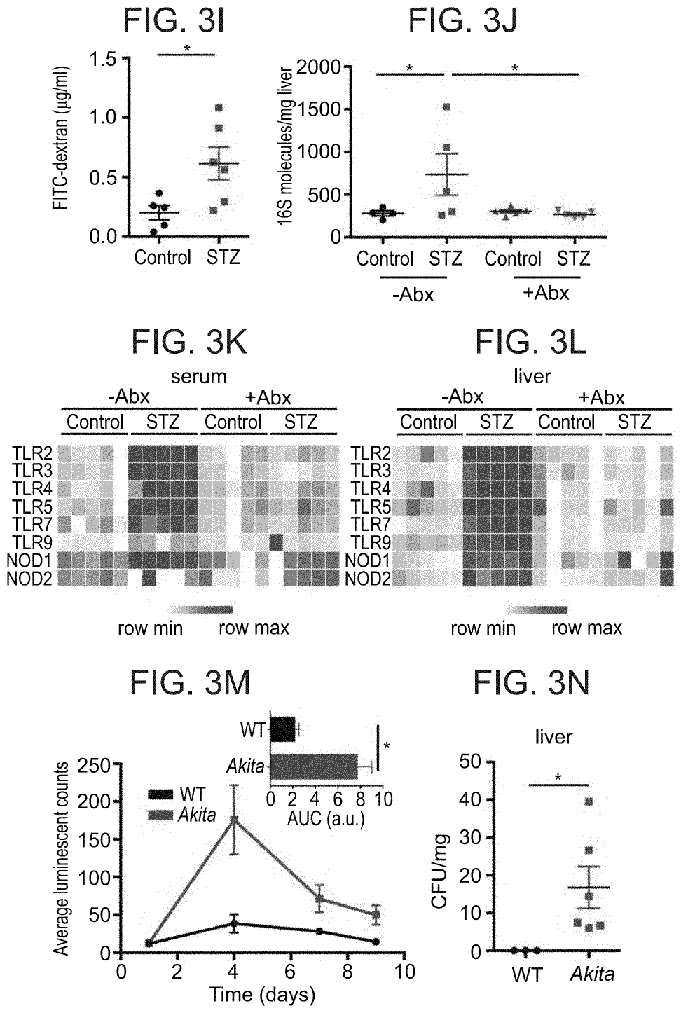

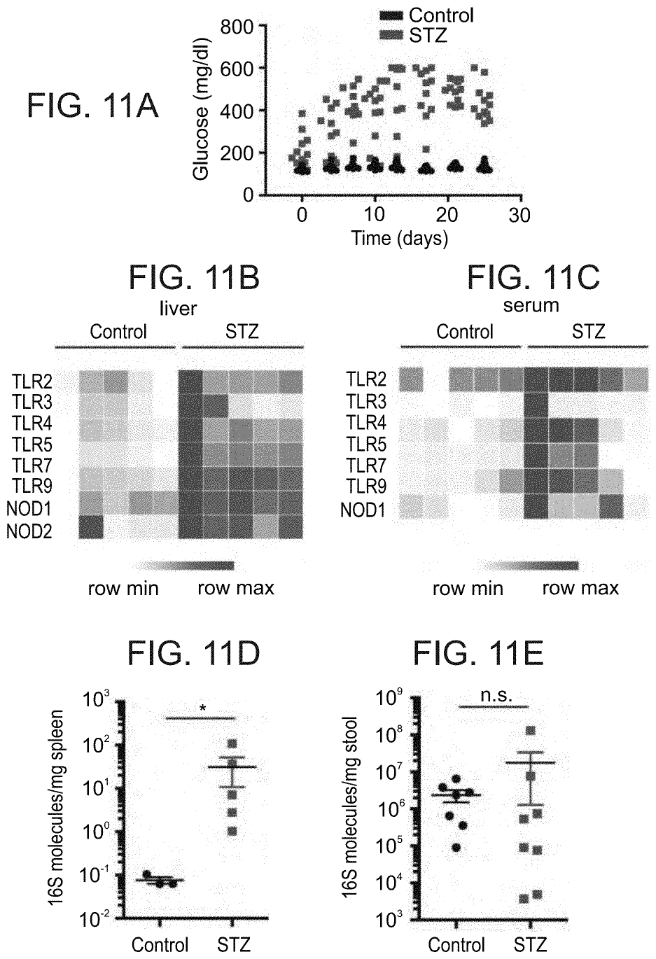

To test whether elevated glucose levels were causally involved in host defense against intestinal infection, hyperglycemia was induced in the absence of obesity in a mouse model of type 1 diabetes mellitus through administration of streptozotocin (STZ (19), FIG. 11A). Indeed, STZ-treated mice developed severe C. rodentium infection and systemic translocation, accompanied by enhanced bacterial growth, epithelial adherence, and systemic spread (FIGS. 3A-E). STZ treatment also resulted in dysfunction of intestinal epithelial adherence junctions under steady-state conditions (FIGS. 3F and 3G), coupled with systemic dissemination of microbial products (FIGS. 11B and 11C), and enhanced trans-epithelial flux (FIGS. 3H and 3I).

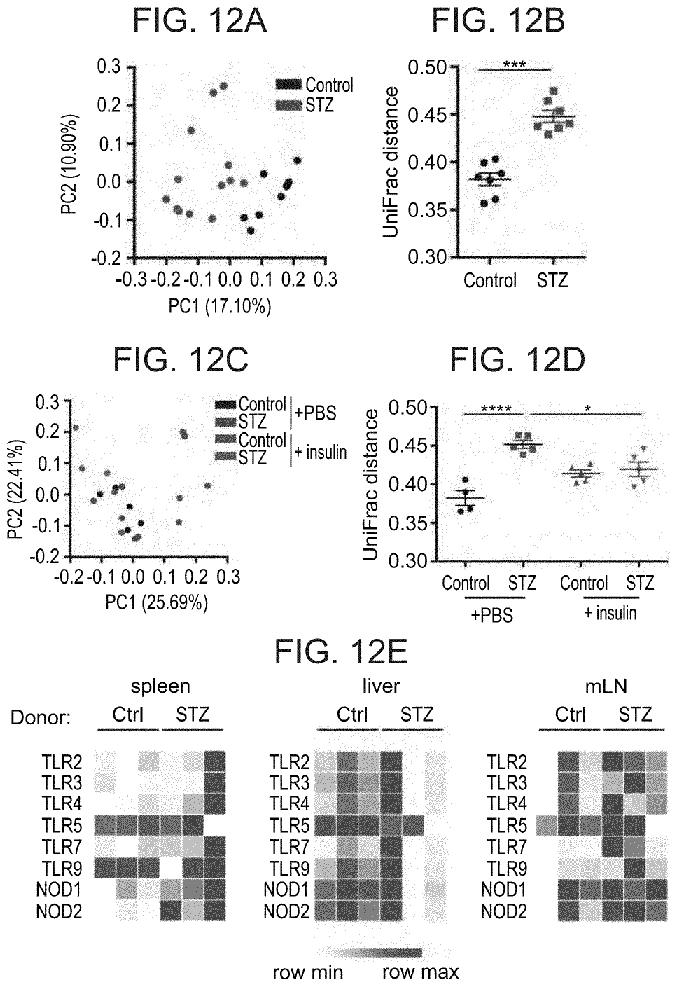

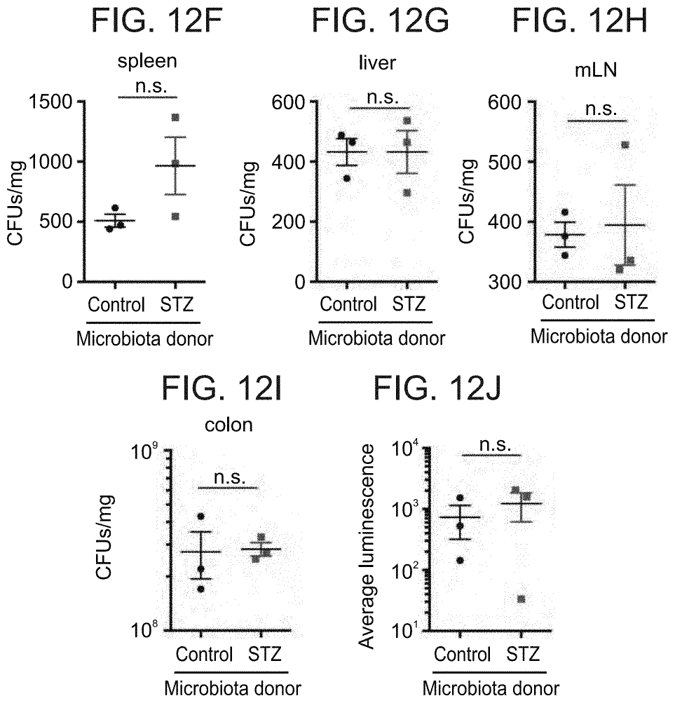

Oral antibiotic treatment prevented the detection of bacterial products at systemic sites in STZ-treated mice (FIGS. 3J-L), demonstrating that the intestinal microbiota was the probable source of disseminated microbial molecules. In contrast to the load of bacterial products at distal organs (FIG. 11D), the microbial load in the intestinal lumen was unaffected by hyperglycemia (FIG. 11E). The present inventors next sought to test the possibility that barrier dysfunction in STZ-treated mice was mediated by compositional microbiota alterations. Indeed, 16S rDNA sequencing revealed a taxonomic change in the configuration of the intestinal microbiota of hyperglycemic mice, which was corrected by insulin treatment and resultant normalization of serum glucose levels (FIGS. 12A-D). However, these compositional microbial changes did not seem to play a critical role in glucose-mediated barrier dysfunction, as microbiota transfer from STZ-treated donors and controls to normoglycemic germ-free mice neither induced dissemination of bacterial products to systemic sites (FIG. 12E) nor increased susceptibility to C. rodentium infection (FIGS. 12F-J). These data indicate that while the commensal microbiota serves as the reservoir of microbial molecules that translocate to the systemic circulation upon disruption of the intestinal barrier, compositional microbiota alterations arising under hyperglycemic conditions do not directly affect barrier integrity.

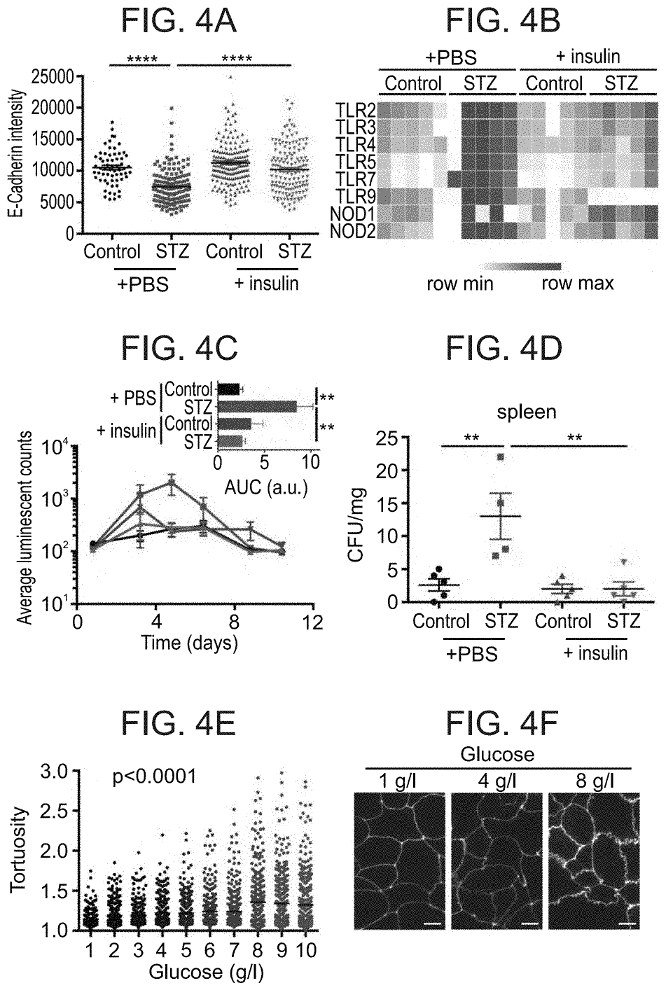

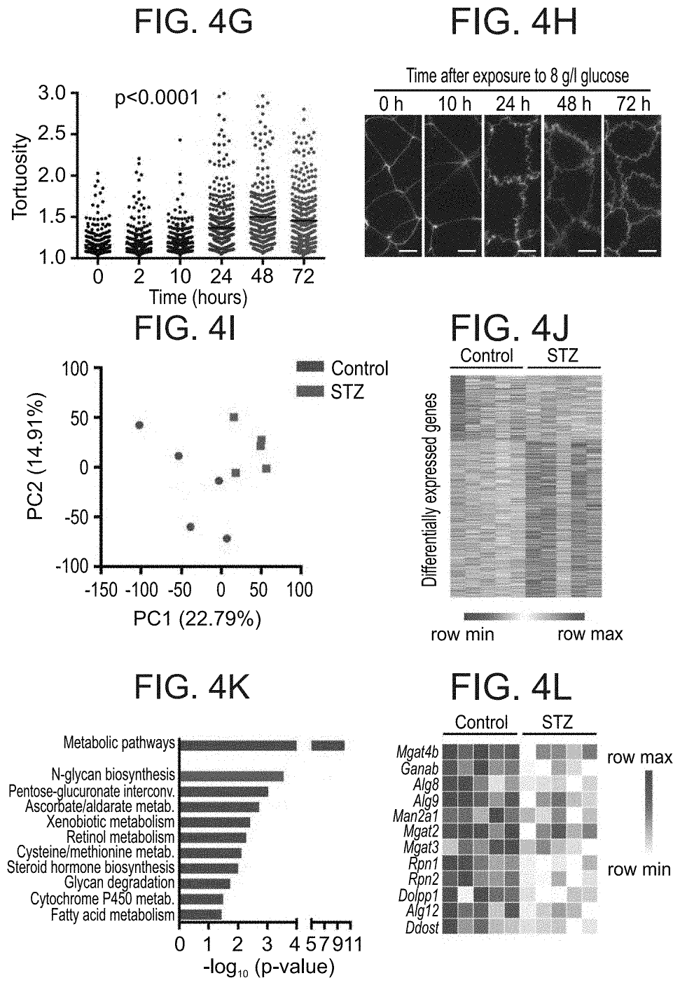

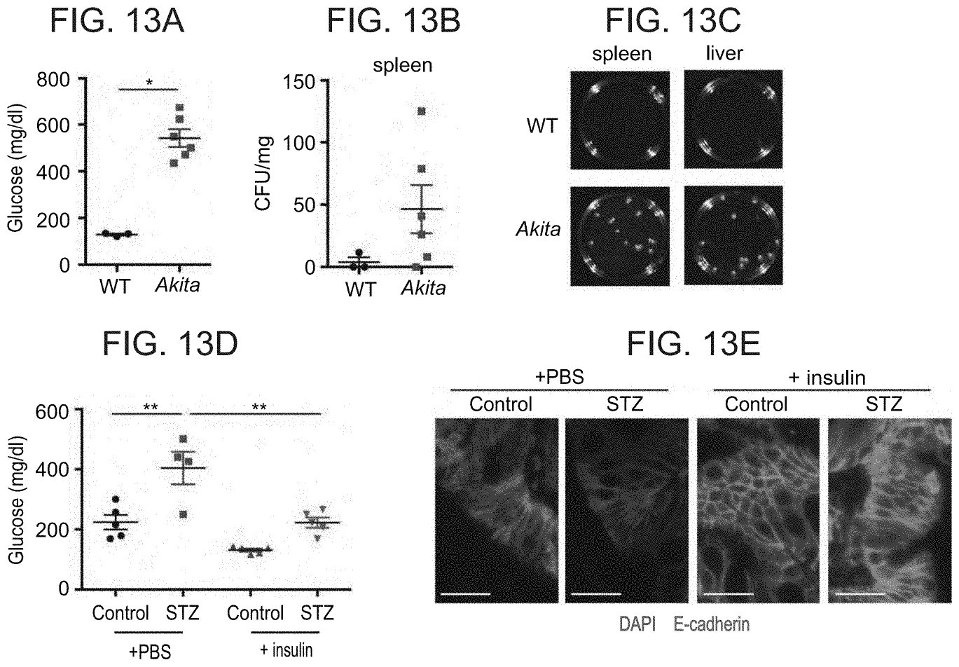

To corroborate the specificity of hyperglycemia as a driver of susceptibility to intestinal infection, hyperglycemic Akita mice were used (FIG. 13A), an STZ-independent model of type I diabetes mellitus that harbors a spontaneous mutation in the insulin 2 gene (20). As in STZ-treated mice, elevated C. rodentium growth and pathogenic translocation to systemic tissues was observed in this model (FIGS. 3M and 3N, and FIGS. 13B and 13C). To further validate the specific impact of hyperglycemia as a driver of the barrier dysfunction phenotype, 0.25 U per day of insulin was administered to STZ-treated mice via hyperosmotic pumps for 4 weeks, which restored normoglycemic levels (FIG. 13D). Treatment with insulin also prevented the loss of adherence junction integrity (FIG. 4A and FIG. 13E), systemic dissemination of microbial products (FIG. 4B), and enhanced C. rodentium growth and pathogenic translocation (FIGS. 4C and 4D). Together, these experiments establish hyperglycemia as a direct and specific cause for intestinal barrier dysfunction and susceptibility to enteric infection.

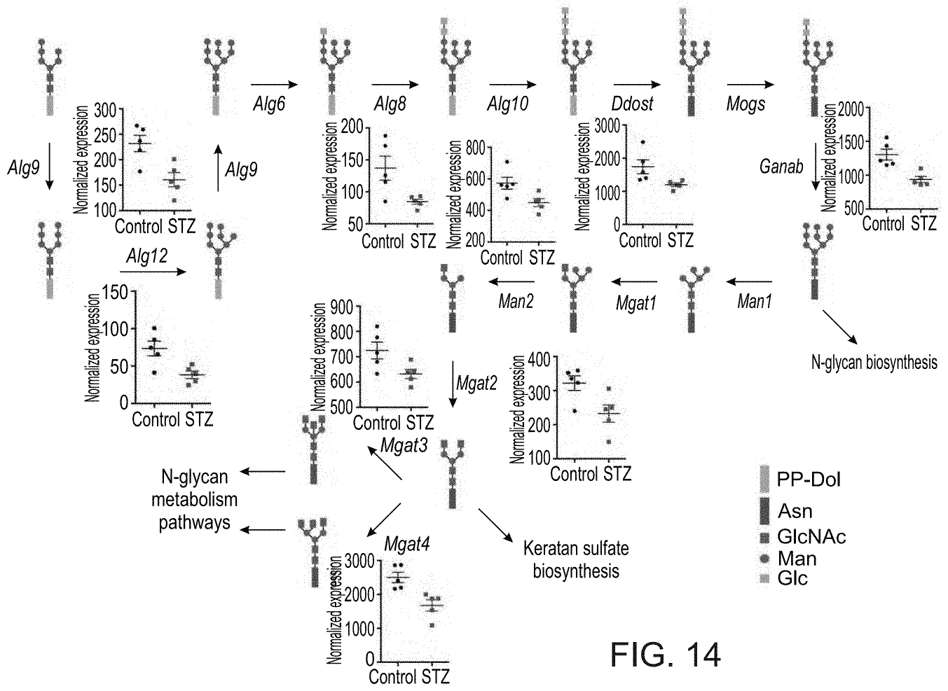

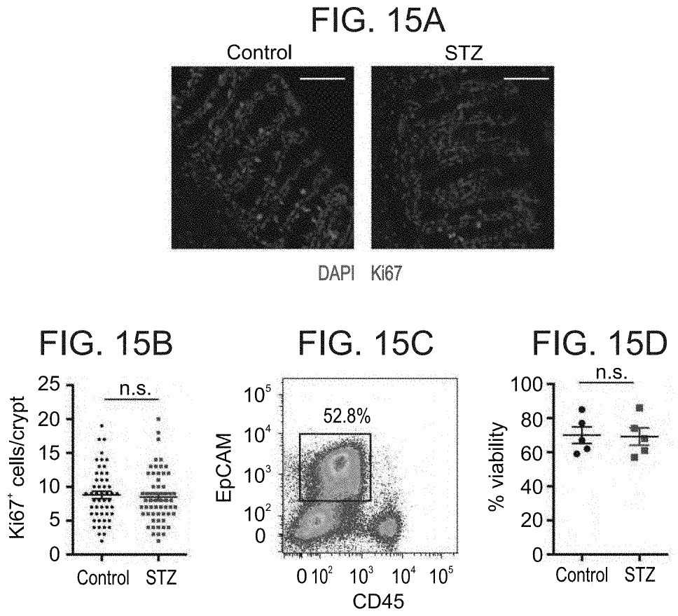

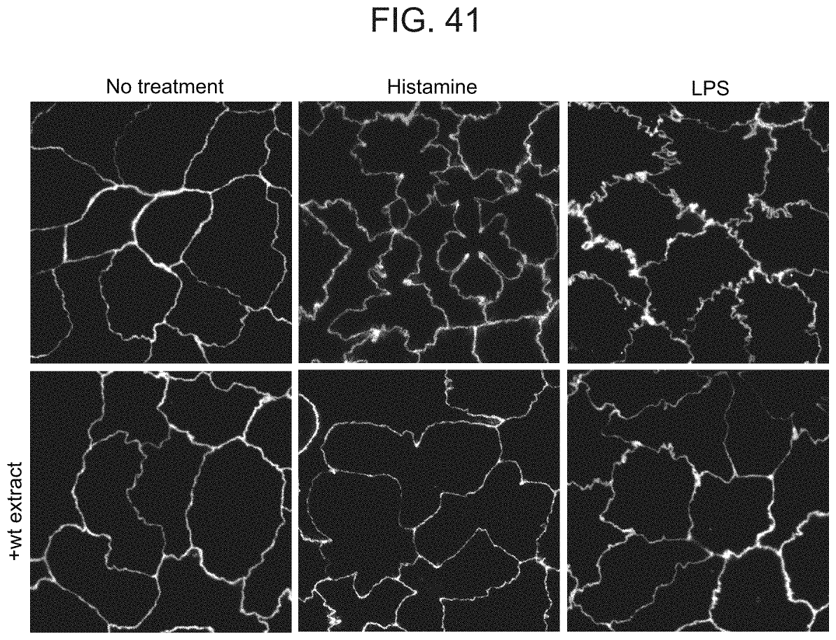

Hyperglycemia Reprograms Intestinal Epithelial Cells To determine whether glucose acted directly on intestinal epithelial cells to affect barrier function, an in vitro system of cultured intestinal epithelial (Caco-2) cells was used. The cells were exposed to different concentrations of glucose in the culture medium. Tight junction integrity was analyzed through automated high-throughput analysis of ZO-1 staining patterns. Indeed, glucose induced barrier alterations in a dose- and time-dependent manner, manifesting visually as increased tortuosity and altered appearance of cell-cell junctions (FIGS. 4E-H). To investigate the mechanisms by which elevated blood glucose levels compromise intestinal epithelial cell function in vivo, RNA-sequencing of purified intestinal epithelial cells from STZ-treated mice and controls was performed. Global reprogramming of the epithelial transcriptome was detected in hyperglycemic mice (FIG. 41), in which more than 1,000 genes were differentially expressed compared to vehicle-treated controls (FIG. 4J). These genes were predominantly involved in metabolic pathways, and specifically in N-glycan biosynthesis and pentose-glucuronate interconversion (FIG. 4K), two intracellular functions critically involved in the maintenance of epithelial barrier function (21-29). For example, hyperglycemia affected the entire pathway of protein N-glycosylation by provoking marked downregulation of central genes (FIG. 4L and FIG. 14). In contrast, epithelial proliferation or cell death was not affected by STZ treatment (FIGS. 15A-D).

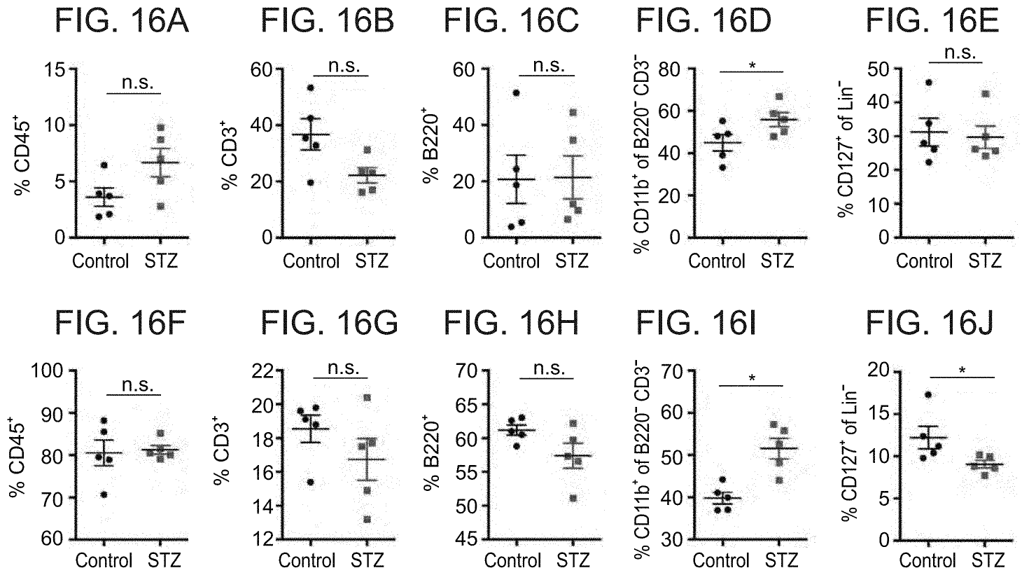

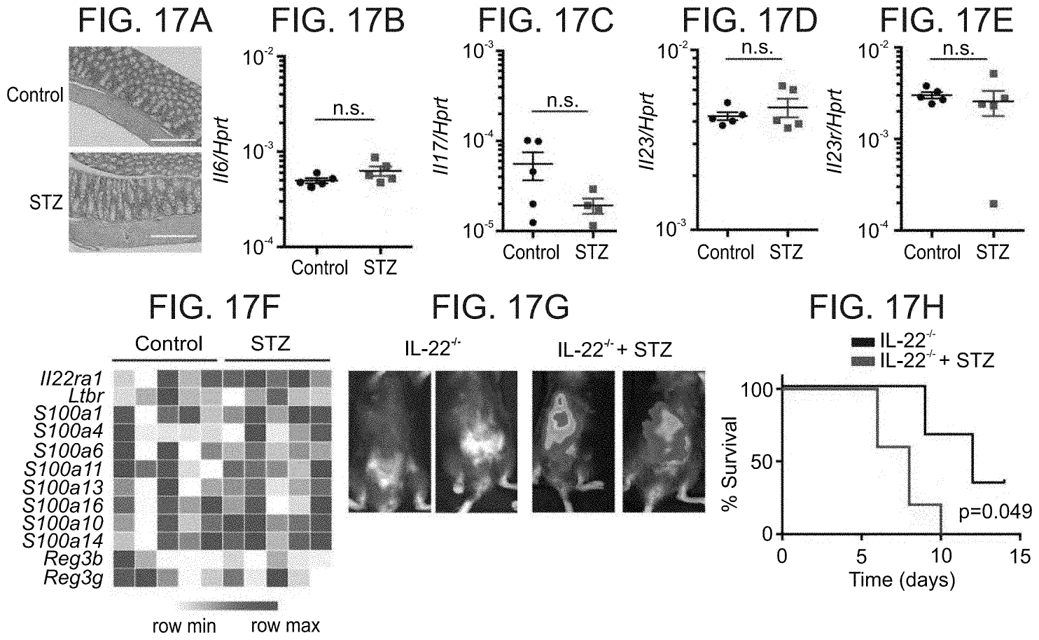

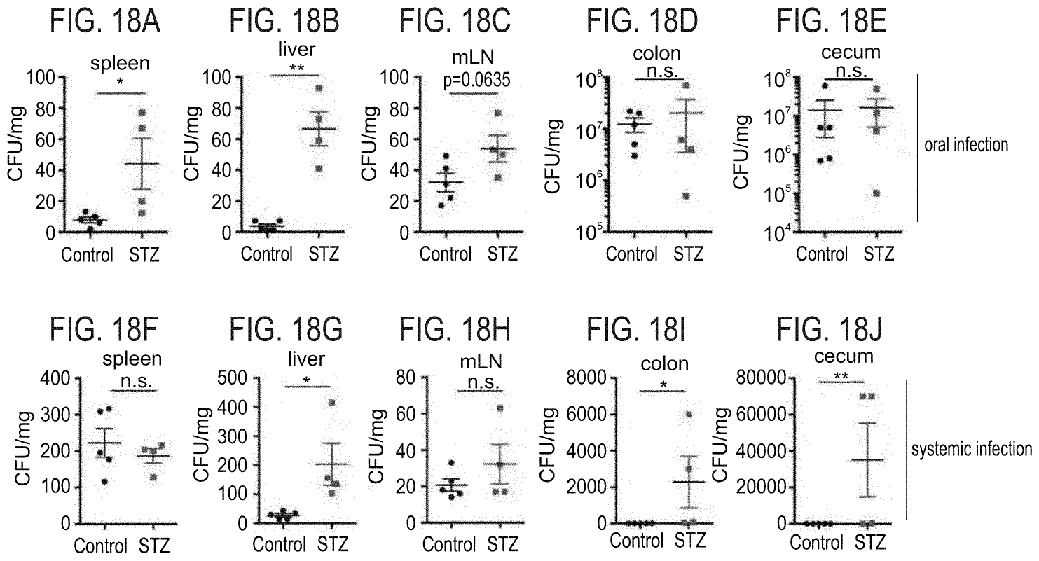

In addition to the above epithelial changes, hyperglycemia modestly affected the intestinal and splenic immune system, specifically by causing an increased representation of myeloid cells (FIGS. 16A-J), in line with previous reports (30). However, STZ treatment did not provoke an overt inflammatory state in the intestine (FIGS. 17A-E). In particular, cytokines involved in IL-22-mediated barrier function and host defense, which has been implicated in the susceptibility of obese mice to infection (12), were unaltered, as was the epithelial transcriptional response to IL-22 (FIG. 17F). In fact, hyperglycemia and IL-22 appeared to have additive effects in mediating host defense against C. rodentium, since STZ-treated IL-22-deficient mice featured accelerated bacterial growth and mortality when compared to IL-22-deficient controls (FIGS. 17G and 17H). The involvement of epithelial and immune cells in host defense against another gastrointestinal pathogen, Salmonella Typhimurium was analyzed. STZ-treated mice orally infected with Salmonella showed enhanced systemic colonization, while intestinal luminal growth was comparable to vehicle-treated controls (FIGS. 18A-E). In contrast to this marked susceptibility of STZ-treated mice to oral Salmonella Typhimurium infection, susceptibility of these mice to systemic infection was only apparent in the liver (FIGS. 18F-H). Interestingly, systemic infection with Salmonella caused enhanced intestinal colonization in STZ-treated mice, potentially indicative of retrograde spread of bacteria across a compromised barrier (FIGS. 181 and 18J).

Epithelial Reprogramming by Hyperglycemia Involves Glucose Metabolism and GLUT2

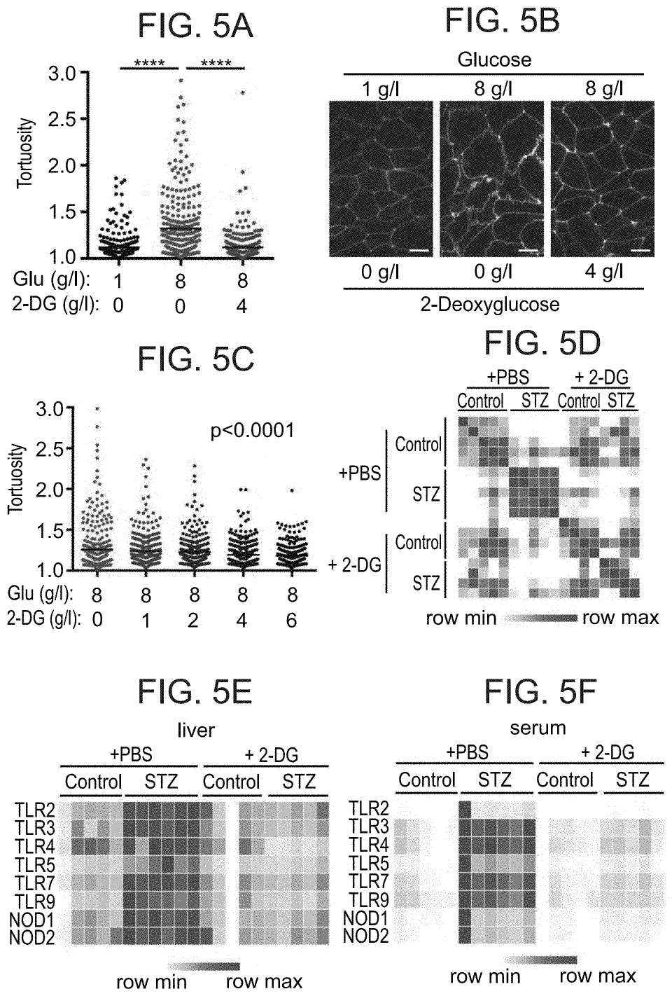

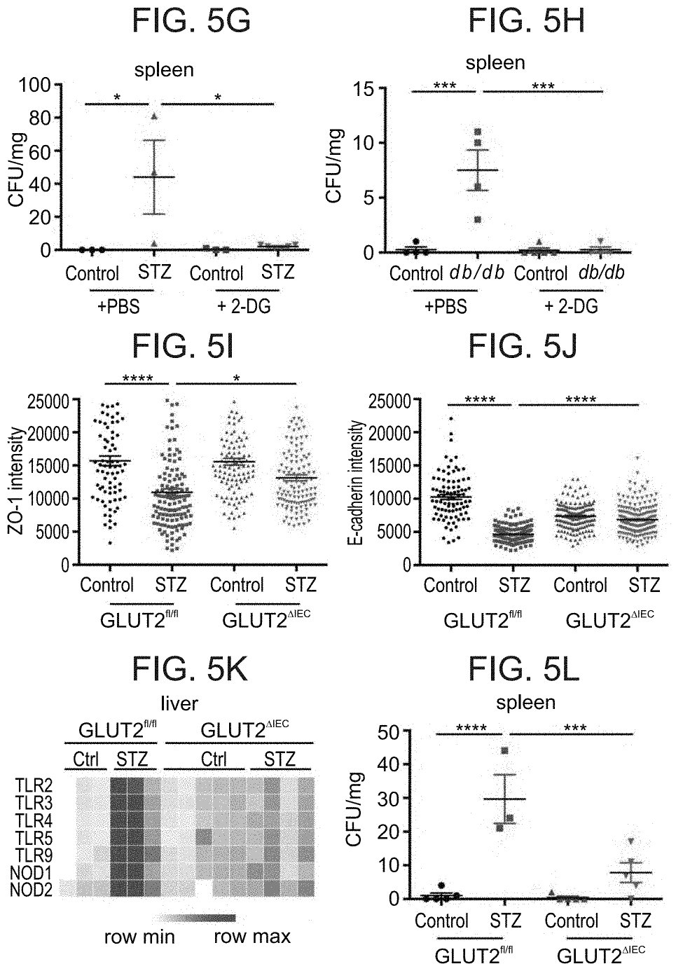

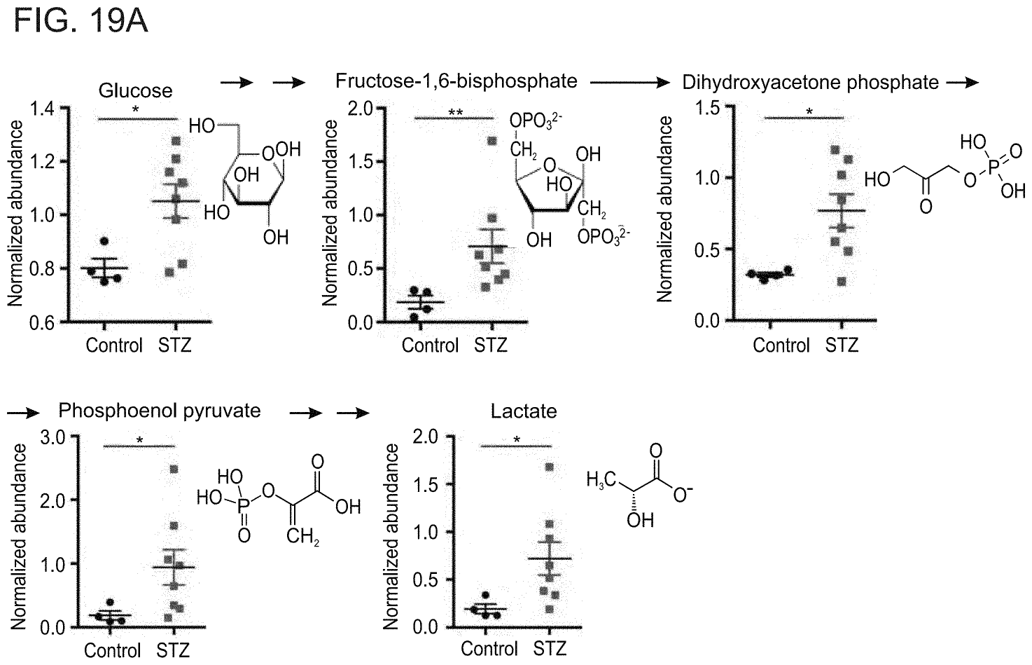

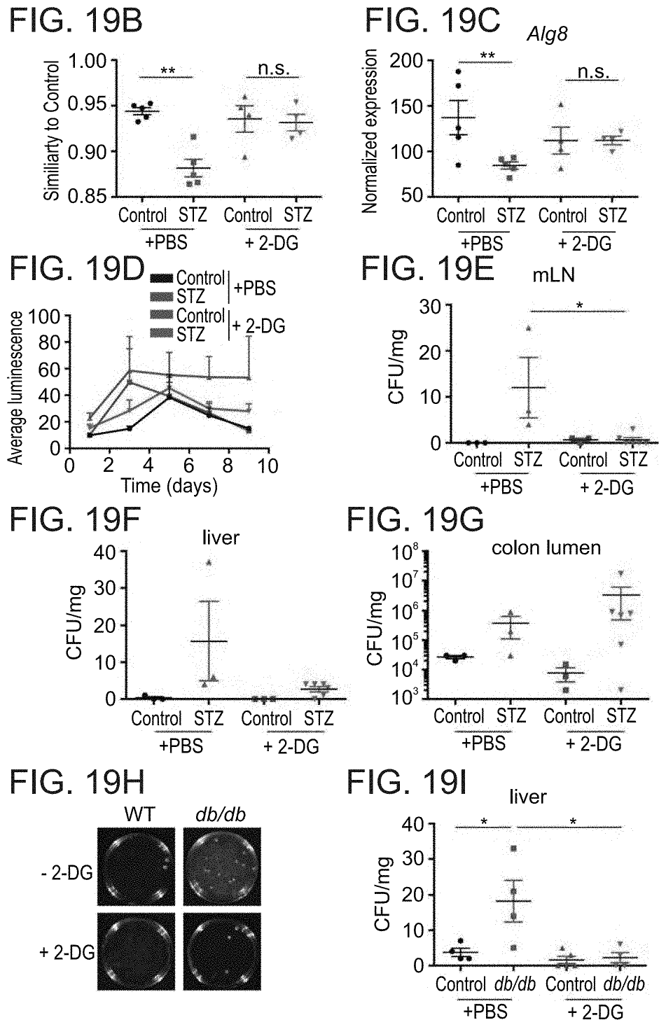

The present inventors next assessed whether epithelial glucose metabolism was involved in the transcriptional reprogramming of STZ-treated mice. Isolated intestinal epithelial cells from hyperglycemic mice featured elevated levels of metabolites along the glycolytic cascade (FIG. 19A). Inhibition of glucose metabolism via 2-deoxyglucose (2-DG), rescued glucose-induced barrier aberrations in vitro in a dose-dependent manner (FIGS. 5A-C). In addition, 2-DG administration blocked transcriptional reprogramming in STZ-treated mice (FIG. 5D and FIG. 19B), including the N-glycan pathway (FIG. 19C), prevented the systemic dissemination of microbial products (FIGS. 5E and 5F), and restored host defense against C. rodentium (FIG. 5G and FIGS. 19D-F). Bacterial growth in the intestinal lumen was unaffected by 2-DG treatment (FIG. 19G). To test whether 2-DG could be used to counteract hyperglycemia-mediated loss of barrier integrity beyond the STZ model, 2-DG was administered to C. rodentium-infected db/db mice and its impact on systemic dissemination of the pathogen was assessed. Notably, the detectable pathogen load in the mesenteric lymph nodes, spleens and livers of 2-DG-treated db/db mice was strongly reduced under 2-DG treatment (FIG. 5H and FIGS. 19H and 191). Together, these data suggest that glucose-mediated reprogramming of epithelial cell metabolic function leads to transcriptional alterations, abrogation of the intestinal barrier, and impaired host defense against enteric infection.

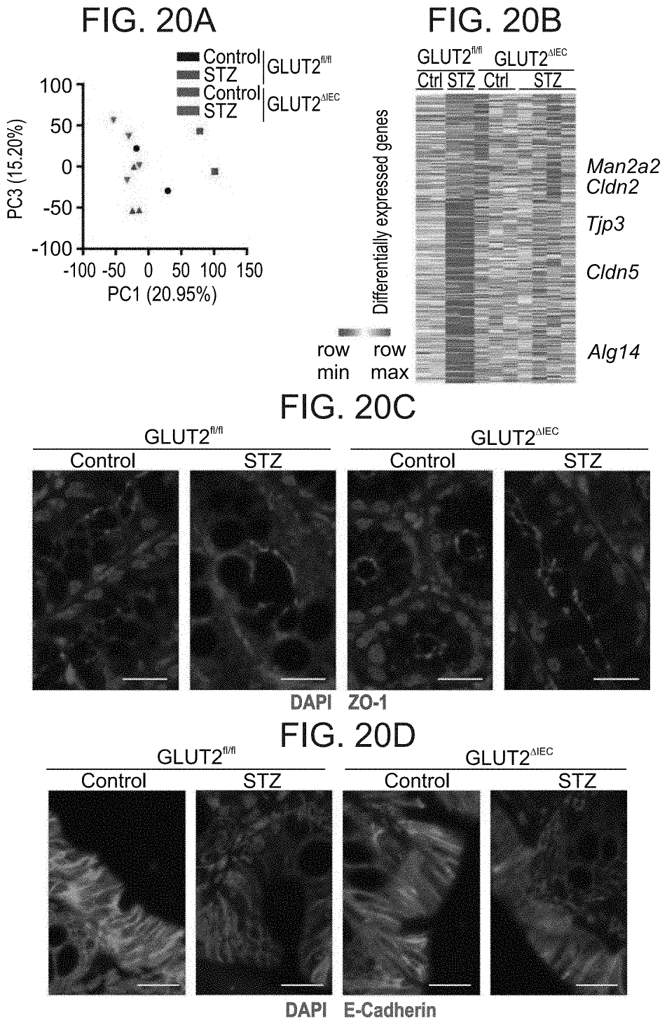

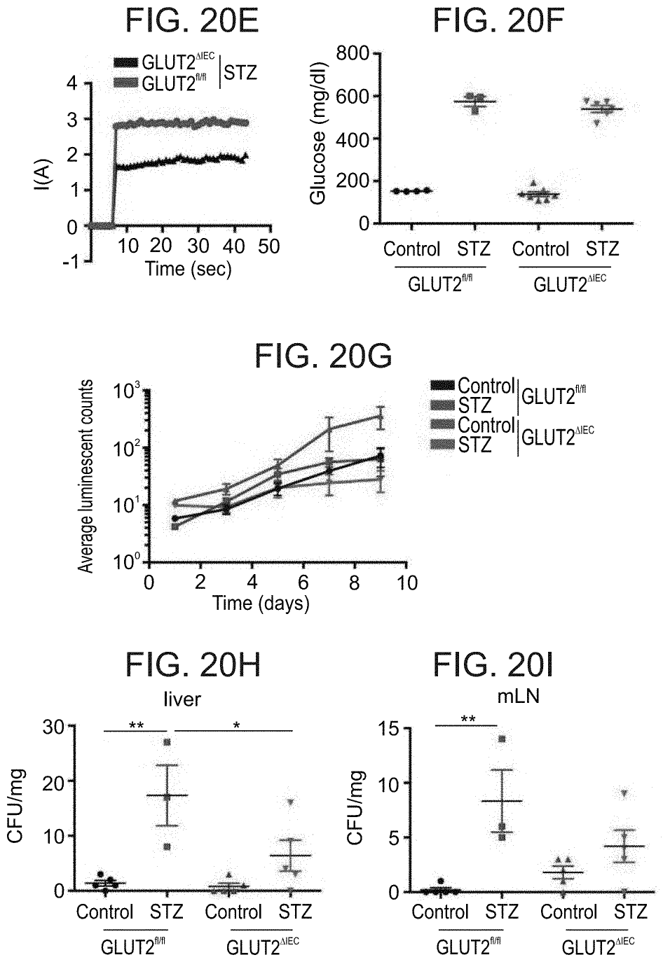

Glucose transport between the intestinal epithelium and circulation is mediated by the bi-directional glucose transporter GLUT2 (31). To determine the role of this transporter in hyperglycemia-mediated epithelial reprogramming, mice selectively lacking GLUT2 in intestinal epithelial cells (GLUT2^{ΔIEC}) (32) were used and hyperglycemia was induced in these mice by STZ administration. Indeed, GLUT2^{ΔIEC} mice were resistant to STZ-induced transcriptional reprogramming and retained epithelial transcriptomes similar to controls (FIGS. 20A and 20B). GLUT2^{ΔIEC} mice also retained intact tight and adherence junction complexes (FIGS. 5I and J, and FIGS. 20C and 20D), reduced transepithelial flux (FIG. 20E), and intestinal containment of microbial PRR ligands (FIG. 5K), despite sustained STZ-induced hyperglycemia (FIG. 20F). Ablation of GLUT2 also ameliorated the STZ-induced susceptibility to C. rodentium growth and systemic dissemination (FIG. 5L, and FIGS. 20G-I). Collectively, these results indicate that GLUT2 is involved in the hyperglycemia-induced metabolic and transcriptional alterations in intestinal epithelial cells, resulting in barrier dysfunction and microbial translocation to the systemic circulation.

Blood Glucose Levels are Associated with Microbial Product Influx in Humans

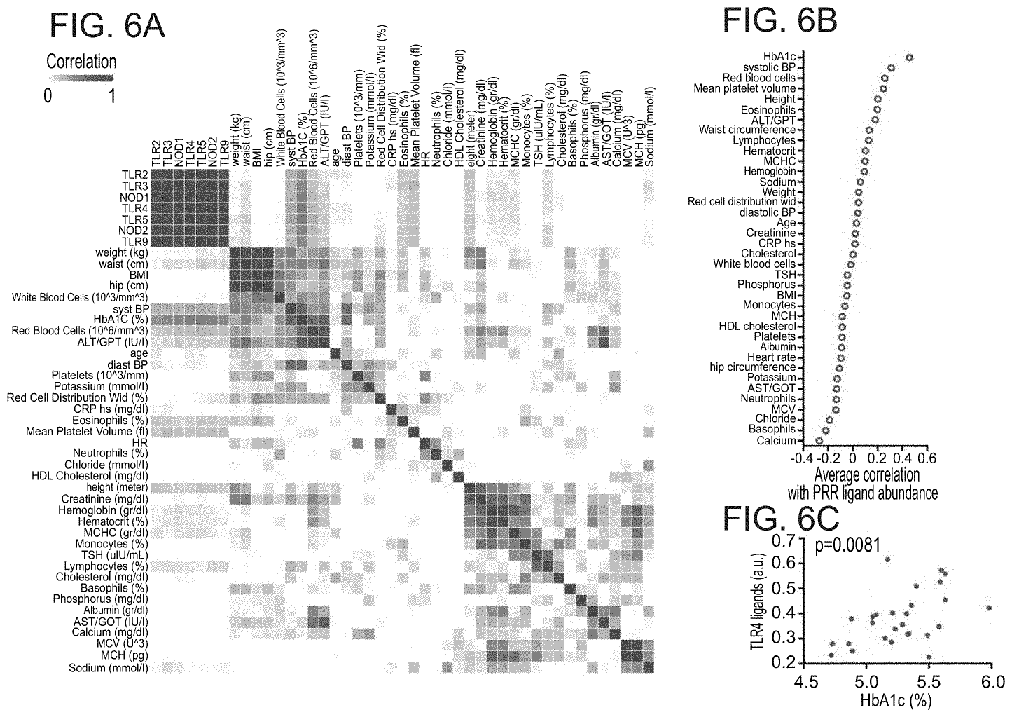

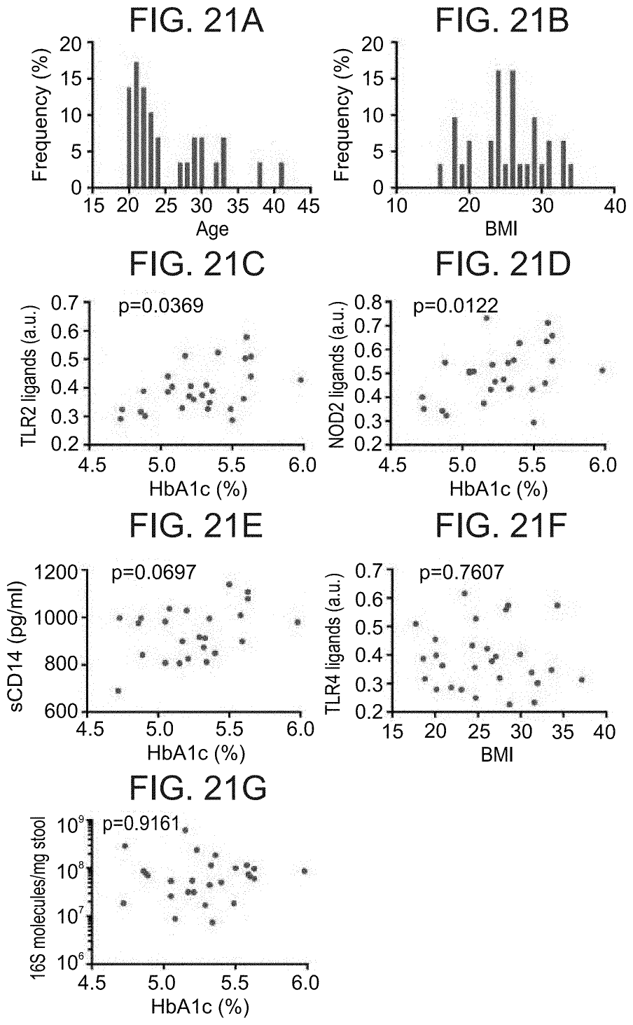

Finally, the present inventors sought to determine whether glycemic levels similarly correlate with intestinal barrier function in humans. To this end, 27 healthy individuals were recruited (FIGS. 21A and 21B) and measurements of multiple serum parameters and microbial products in the circulation were taken. Of all variables measured, hemoglobin A1c (HbA1c), indicative of an individual's three-month average plasma glucose concentration, showed the strongest correlation with serum levels of PRR ligands (FIGS. 6A-C, and FIGS. 21C-E). In contrast, high BMI and other hallmarks of metabolic disease did not significantly associate with the influx of microbial products (FIGS. 6A and 6B, FIG. 21F). Total stool bacterial content did not correlate with HbA1c levels (FIG. 21G). Together, these data suggest that similarly to mice, serum glucose levels, rather than obesity, may associate with or potentially even drive intestinal barrier dysfunction in humans.

### Example 2

### Development of an Ex-Vivo IBD Model, Enabling High Throughput Screening for Modulators of Tight Junction Integrity

Materials and Methods

Cell Culture:

CaCo-2 cells were purchased from ATCC. Cells were routinely grown in regular tissue culture medium (DMEM, 10% FCS, penicillin/streptomycin, GlutaMAX). For experiments with differing glucose concentrations, cells were routinely cultured in low-glucose DMEM (1 g/l glucose). Cells were seeded onto 96-well glass-bottom plates coated with either fibronectin or collagen 70 000 cells/well and allowed to grow for 24 hours to form confluent monolayer before indicated treatments.

Cell Substrate Preparation:

Collagen gels were prepared from 3 mg/ml rat tail collagen solution (Roche) via cross-linking with 1-ethyl-3-(3-dimethylaminopropyl) carbodiimide (EDC) and N-hydroxysuccinimide (NHS) as previously described (Revach et al. Exp Cell Res. 2016). 20 μl of collagen/EDC/NHS mixture were applied on the bottom of each well in 96 well coverglass bottom plate, allowed to polymerize overnight at room temperature, equilibrated with cell culture medium for 2 hours at 37° C. before cell plating. For fibronectin coating, multiwell coverglass bottom plates were incubated with 50 μl per well of bovine fibronectin (Biological Industries) solution 25 μg/ml in PBS for 1 hour at 37° C. and rinsed 3 times with PBS before cell plating.

Cell Labeling:

For immunofluorescent staining, cells were permeabilized with 0.5% Triton ×100 in 3% paraformaldehyde for 3 minutes and fixed with 3% paraformaldehyde for an additional 30 minutes. Tight junction labeling was performed with either rabbit anti-cingulin antibodies or with anti-zo1 mouse monoclonal antibodies (BD Transduction Laboratories). Focal adhesions were labeled with either anti-paxillin mouse monoclonal antibodies (BD Transduction Laboratories) or with anti-zyxin rabbit polyclonal antibodies. Adherens junctions were labeled with anti-β-catenin rabbit polyclonal antibodies (Sigma).

The following secondary, fluorescently labeled antibodies (Invitrogen) were used: Alexa Fluor 488 goat anti-mouse or anti-rabbit antibodies for tight junction visualization and Alexa Fluor 647 goat anti-mouse or anti-rabbit antibodies for focal adhesions or adherens junctions visualization. Actin was labeled with TRITC-phalloidin (Sigma) and nuclei with DAPI.

Imaging:

For high resolution images of manually selected representative fields, a DeltaVision wide field fluorescent microscope (GE Healthcare) equipped with 40×UPlanFLN or 60×/1.42 PlanApo N oil objective (Olympus) was used. For multi-well plate scanning, the following automated imaging stations were used: DeltaVision microscope with plate scanning regiment, Hermes imaging station (IDEA bio-Medical) or ImagExpress microXL imaging station (Molecular Devices) equipped by 40× air objectives. In all cases in order to cover the curvature of apical surface of epithelial monolayer 5 μm thick z-stack was acquired around the tight junction plane (identified by best focus algorithm) and resulting image was generated as a maximum projection of all optical sections.

Cell images were segmented using Morphological Segmentation ImageJ plug-in and junction tortuosity was calculated as a ratio between junction length and Euclidian distance between its ends.

Traction Force Microscopy:

SoftTrac 6 well plates with pre-made collagen coated 12 kPa hydrogels containing embedded 0.2 μm fluorescent beads were purchased from Matrigen. 200,000 cells per well were plated and allowed to grow for 24 hours to form epithelial islands, treated with the indicated metabolite for another 24 hours and live-imaged using DeltaVision microscope equipped with environmental chamber. After acquisition of cell and fluorescent bead images in steady state conditions, cells were detached with trypsin and imaging of beads was repeated in a relaxed state. Displacement map generation and force calculations were performed using MatLab script described in Butler et al. Am J Physiol Cell Physiol. 2002.

Robotic Experimental Design for High Throughput Screening:

2956 biologically active compounds (SelleckChem) were tested for their ability to damage or to protect tight junctions organization. In order to create disease-mimicking conditions, cells were treated with culture medium containing 300 ng/ml LPS and 10 mM Histamine (bacterial surface antigen and bacterial metabolite) in combination with bioactive compounds.

Cell plating, drug treatment, fixation and labeling procedures in high throughput format were performed using the following equipment:
(i) Multidrop Combi Reagent Dispenser (Thermo Fisher Scientific) was used for fibronectin coating, cell dispensing, primary and secondary labeling mixture dispensing.
(ii) Bravo Automated Liquid Handling Platform (Agilent) was used for addition of drug-containing medium, permeabilization with PFA/Triton and PFA fixation.
(iii) EL406 Washer Dispenser (BioTek) was used for liquid aspiration and washes between fixation and labeling steps.

Results

Effect of IBD-Related Cytokines and Metabolites in TJ Integrity

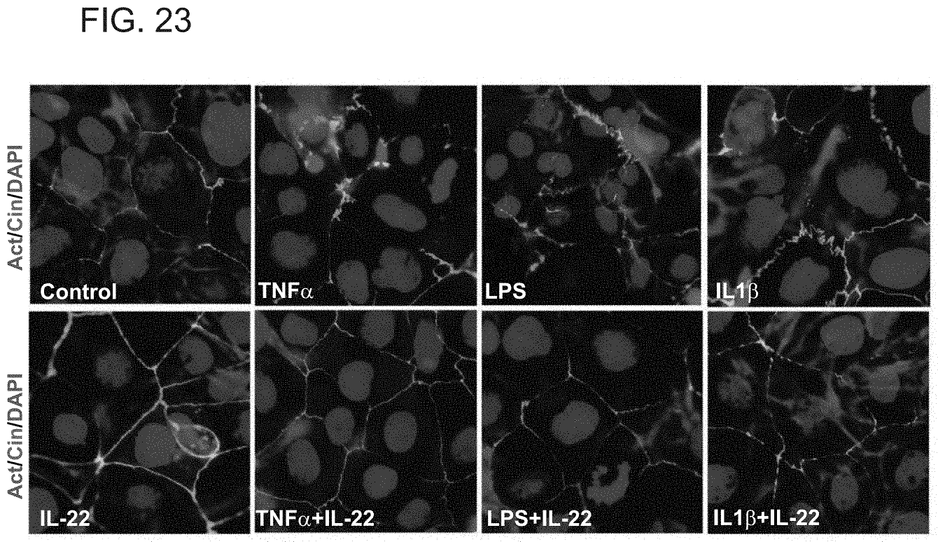

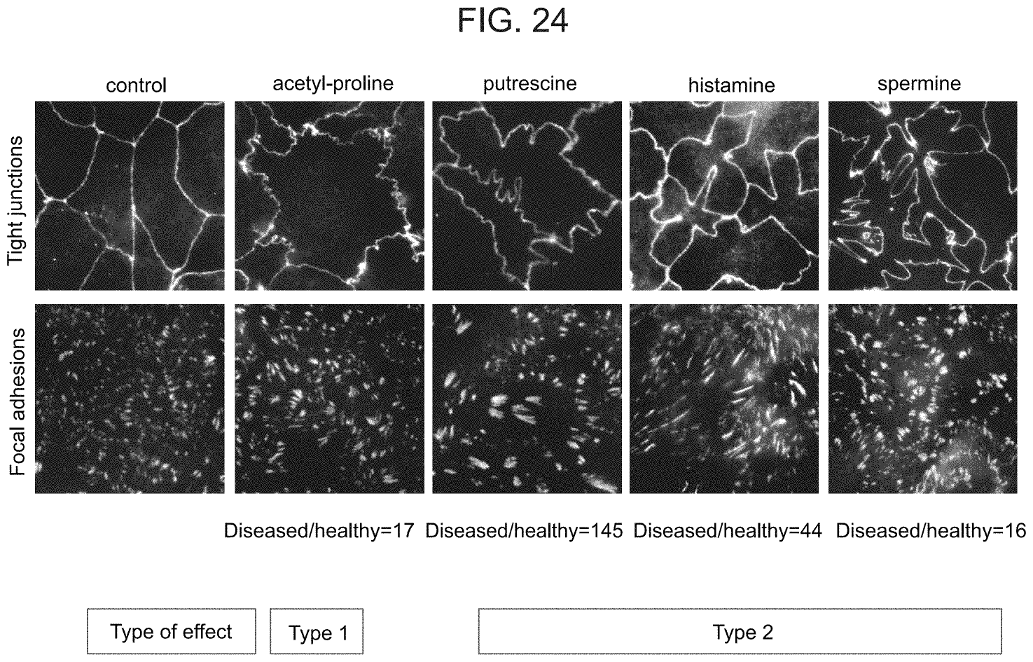

Caco2 cells were treated with several known IBD mediators of host and bacterial origin. Treatments included: cytokines TNFα and Il-1β, bacterial surface antigen LPS (FIG. 23), and the bacterial metabolites histamine and spermine (FIG. 24). In these proof-of-concept experiments, the morphological effects of known IBD mediators were characterized. Two main phenotypes were identified, referred to as type 1 (“zipper-like”, see TNFα and LPS treatments), and type 2 (“flower-like”, see histamine and spermine treatments).

Simultaneous treatment with type 1 and 2 disruptors results in a combined tight junction phenotype.

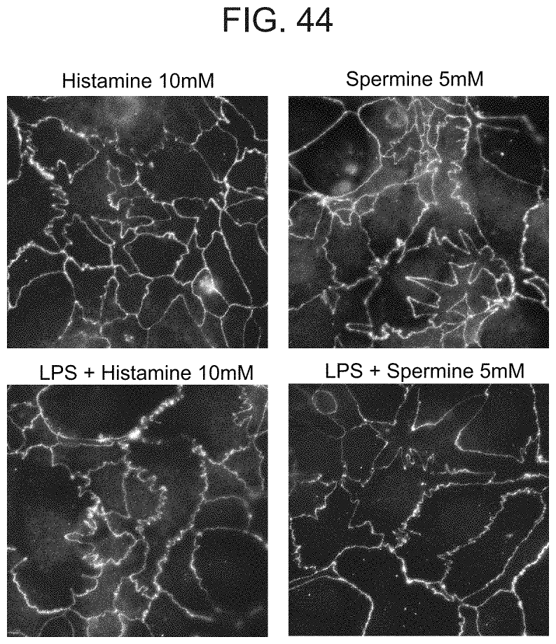

Caco2 cells were treated with either histamine or spermine alone or in combination with LPS. Histamine and spermine induce dramatic change in epithelial geometry and “flower-like” phenotype—see FIG. 44.

Combination of either histamine or spermine with LPS caused a double effect—change in cell geometry (type 2 disruption) and appearance of zigzag shaped tight junctions (type 1 disruption)—see FIG. 44.

FIG. 45 illustrates that the disruptive effect of histamine on epithelial tight junctions is transduced via type 2 histamine receptor.

In addition to IBD mediators, two molecules which in vivo exhibit anti-inflammatory and anti-colitogenic properties were tested: human cytokine IL-22 (FIG. 23) and bacterial metabolite taurine (FIG. 24) in combination with the above-mentioned epithelial disruptors.

As seen in FIG. 23, a very tight correlation between junction rupture ex vivo and IBD in vivo was found. IBD-related cytokines disrupted TJ, and this effect was blocked by the non-inflammatory cytokine IL-22.

Secreted Molecule Screen for Tight Junction (TJ) and Focal Adhesion (FA) Modulators

A protein library, consisting of 295 human secreted proteins was used for identification of potential modulators of the barrier function of human intestinal epithelial cells.

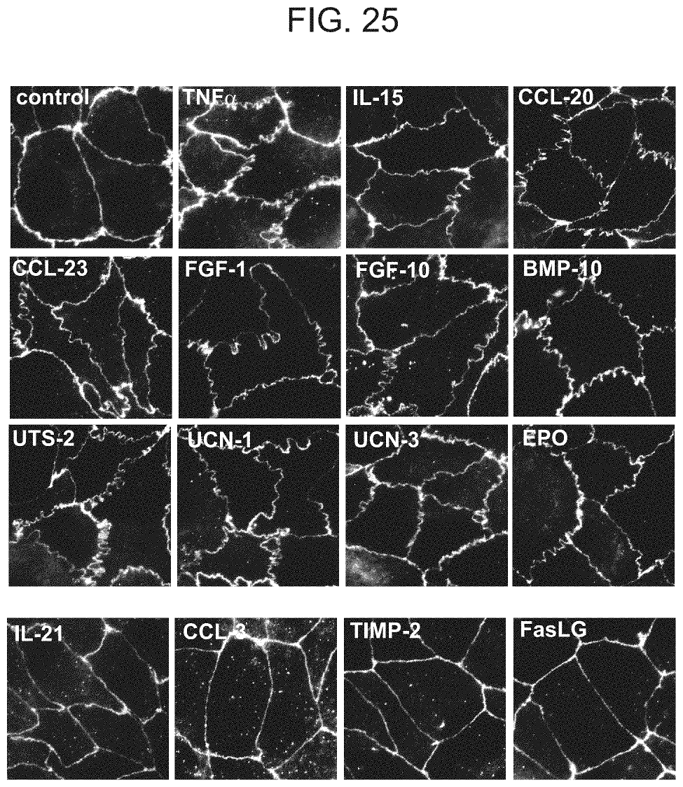

11 secreted molecules were shown to induce loss of normal structure of tight junctions. 4 secreted proteins displayed a “stabilizing” phenotype, with uniform and smooth TJs, and a capacity to inhibit the effect of selected disruptors (FIG. 25).

The full list of disruptors and stabilizers identified in human secreted molecules library is presented in Tables 5 and 6, herein below.

TABLE 5

| molecule | Epithelial disruptors | functional group |
|---|---|---|
| IL-15 | Interleukin-15 | mediators of inflammation |
| CCL-20 | Chemokine ligand 20 | |
| CCL-23 | Chemokine ligand 23 | |
| FGF1 | Fibroblast growth | regulators of proliferation |
| | factor 1 | and morphogenesis |
| FGF10 | Fibroblast growth factor 10 | |
| BMP10 | Bone morphogenetic protein 10 | |
| UTS2 | Urotensin 2 | Vasoconstrictor |
| UCN1 | Urocortin 1 | neuropeptides |
| UCN3 | Urocortin 3 | |
| EPO | erythropoietin | inductor of erythropoiesis |

| molecule | Epithelial stabilizers | functional group |
|---|---|---|
| IL-21 | Interleukin-21 | mediators of inflammations |
| CCL-3 | Chemokine ligand 3 | |
| TIMP-2 | Tissue metalloprotease | Regulator of |
| | inhibitor 2 | ECM remodelling |
| FasLG | Fas ligand | Inductor of apoptosis |

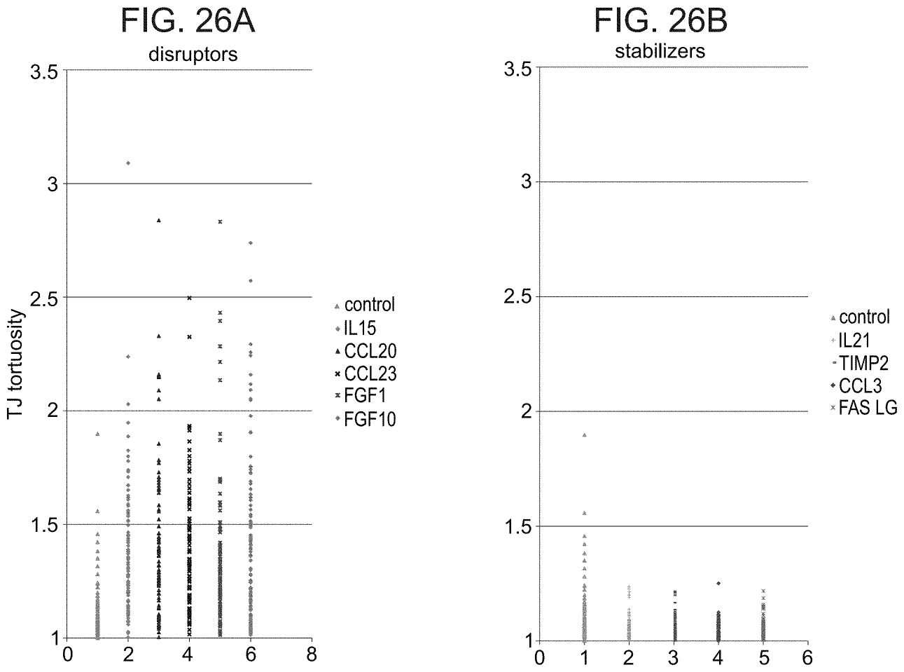

Quantification of TJ morphology upon treatments leading to disruption or stabilization is presented in FIGS. 26A-B.

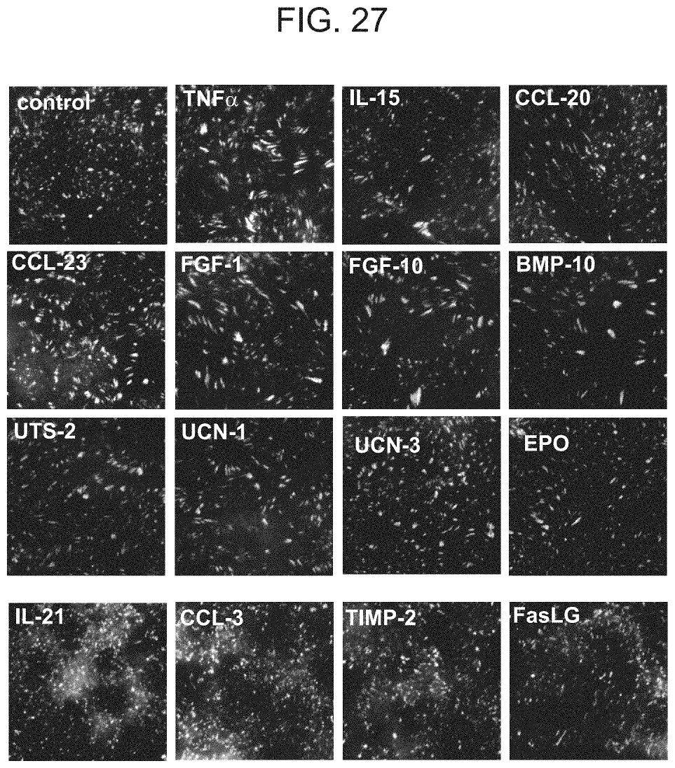

Examination of the basal aspects of the tested cells (same cells as shown in FIG. 25), revealed that the organization of focal adhesions to the ECM were inversely correlated to the effect on TJ integrity—FIG. 27. Thus, treatments that disrupted TJs, augmented FA formation, and those that enhanced TJ stability, disrupted FA formation. This observation is in line with the interesting possibility that loss of epithelial hallmarks in IBD might be driven by epithelial-mesenchymal transition (EMT), a common process associated with cancer progression, whereby TJs and adherens junctions (cadherin dependent) are down-regulated, while mesenchymal features become more apparent. This is further in line with the reduction in FA formation, induced by all TJ-stabilizers, suggesting a possible “MET effect (mesenchymal-epithelial transition).

Metabolite Screen for TJ Focal Adhesion (FA) Modulators

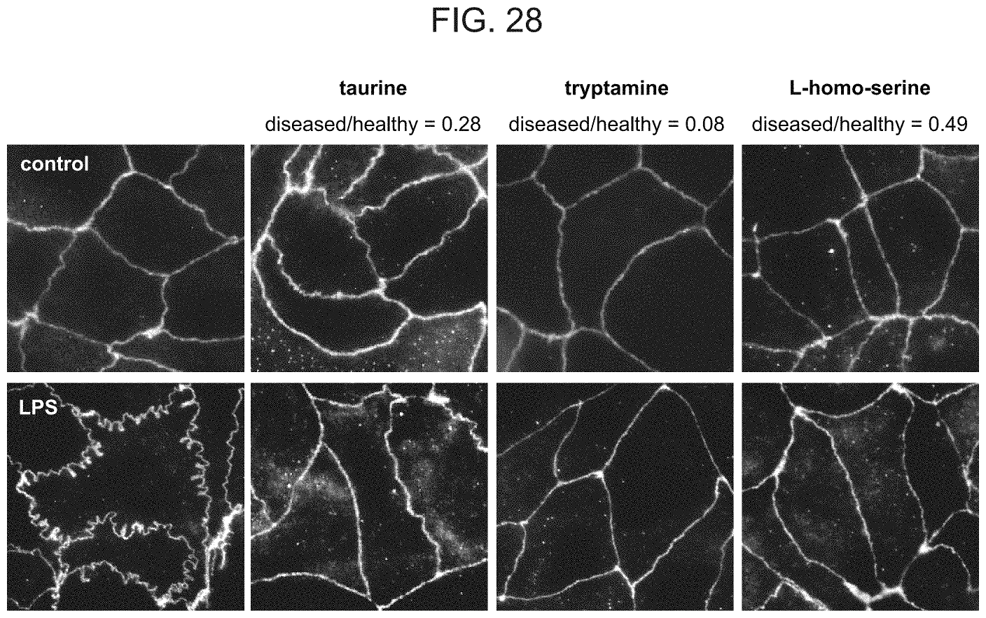

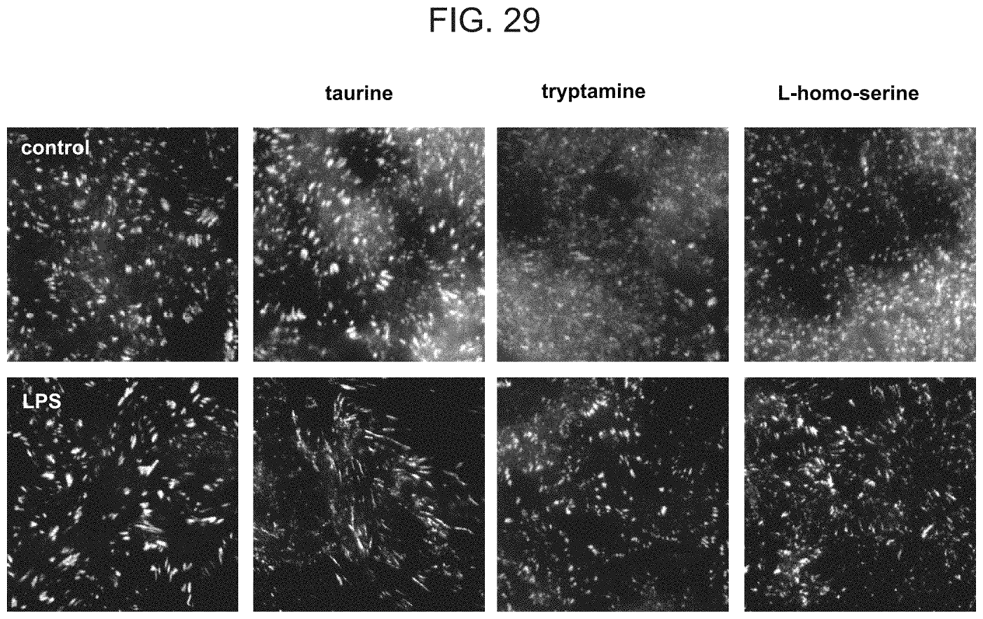

Metabolites used in this screen were based on the relative prominence of specific molecules in healthy and diseased mice (Levy M. et al. Cell 2015). 4 disrupting and 3 stabilizing molecules were identified—see FIGS. 24, 28 and 29 and Tables 7 and 8, herein below.

TABLE 7

| | Epithelial disruptors | diseased/ | |
|---|---|---|---|
| Molecule | Biosynthetic pathway | healthy ratio | |
| putrescine | Polyamine Metabolism | 145.2 | Disruption |
| histamine | Histidine Metabolism | 44.18 | Disruption |
| spermine | Polyamine Metabolism | 15.7 | Disruption |
| N-acetylproline | Urea cycle; | 16.58 | Disruption |
| | Arginine and | | |
| | Proline Metabolism | | |

TABLE 8

| | Epithelial stabilizers | diseased/ | |
|---|---|---|---|
| Molecule | Biosynthetic pathway | healthy ratio | |
| tryptamine | Tryptophan Metabolism | 0.08 | Stabilization |
| taurine | Methionine, Cysteine, | 0.28 | Stabilization |
| | SAM and | | |
| | Taurine Metabolism | | |
| L-homoserine | Glycine, Serine | 0.49 | Stabilization |
| | and Threonine | | |
| | Metabolism | | |

Interestingly, disrupting and stabilizing metabolites caused changes in cell-matrix focal adhesions (FA) similar to those observed upon treatment with disrupting and stabilizing human secreted molecules, i.e. disruption of tight junctions correlated with increase of focal adhesions while TJ stabilization coincided with FA reduction (FIGS. 24, 28 and 29).

Compound Library (SelleckChem, Bioactive Compound Library) Screen for TJ Modulators

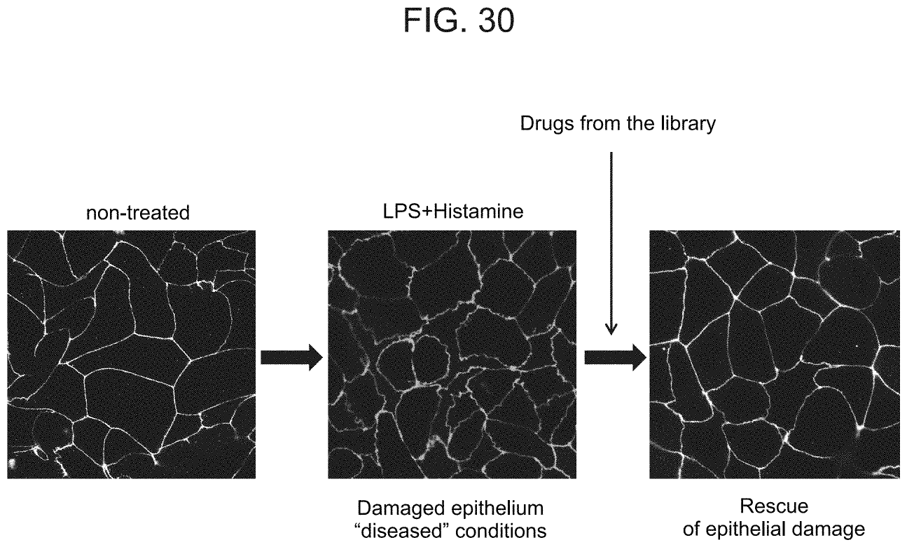

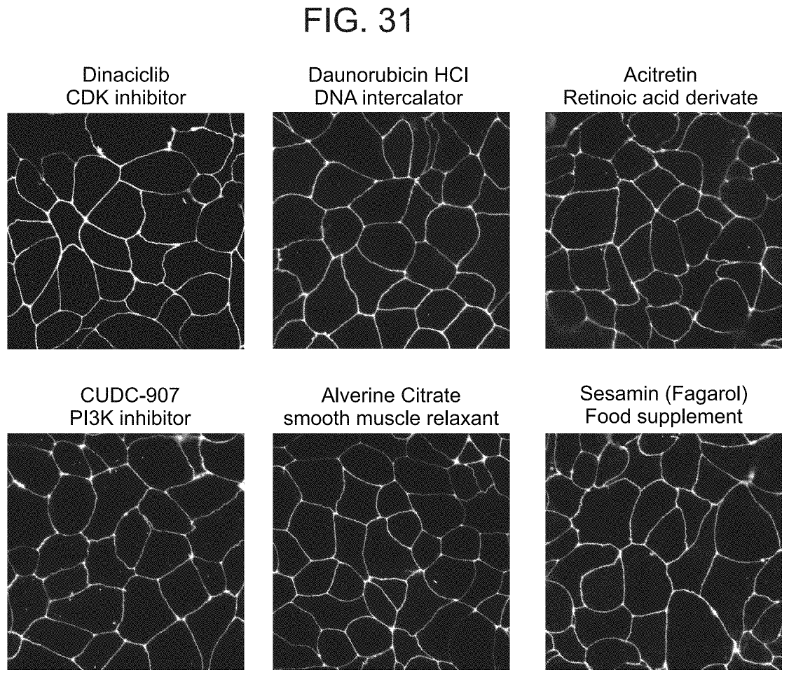

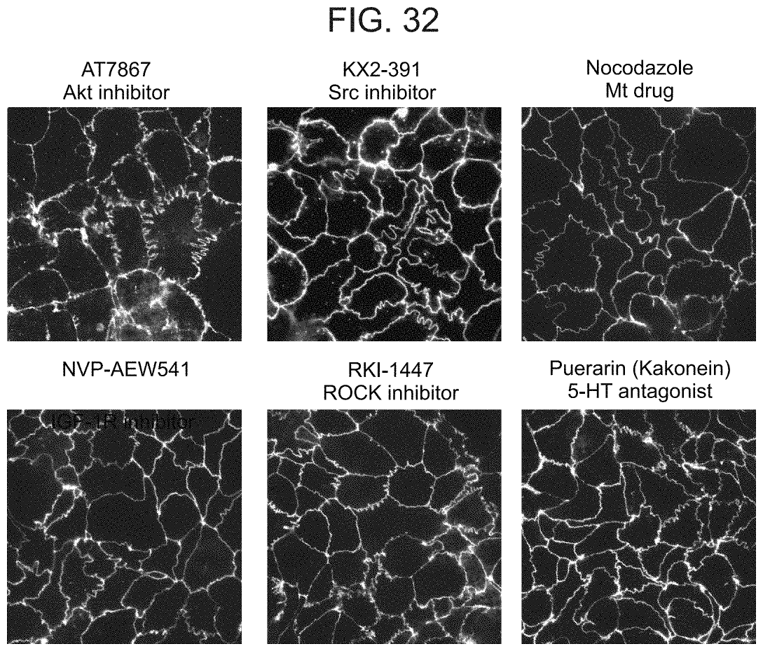

The Selleck Chemical library with >2,500 compounds, many of which are FDA-approved drugs or broadly characterized inhibitors of major intracellular signaling pathways was tested on Caco2 cells that were treated with a combination of disruptors in order to create disease mimicking conditions. FIG. 30 illustrates how the assay was carried out. Table 9 summarizes the compounds that disrupted tight junctions and Table 10 summarizes the compounds that stabilize tight junctions. Representative results are provided in FIGS. 31 and 32.

TABLE 9

| | |
|---|---|
| MLN8237 (Alisertib) | Aurora Kinase inhibitor |
| Danusertib | Aurora Kinase, Bcr-Abl, c-RET, FGFR |
| (PHA-739358) | inhibitor |
| Rigosertib (ON-01910) | PLK/Src inhibitor |
| KX2-391 | Src inhibitor |
| AT7867 | Akt inhibitor |
| GDC-0068 | Akt inhibitor |
| LY2109761 | TGFbeta inhibitor |
| PF-04691502 | PI3K/mTOR inhibitor |
| TG101348 | JAK |
| (SAR302503) | inhibitor |
| CEP33779 | JAK inhibitor |
| LY2784544 | JAK inhibitor |
| Baricitinib (LY3009104) | JAK inhibitor |
| NVP-AEW541 | IGF-1R inhibitor |
| NVP-ADW742 | IGF-1R inhibitor |
| PHA- | CDK + Thropomyosin receptor |
| 848125 | kinase A inhibitor |
| CI994 (Tacedinaline) | HDAC inhibitor |
| GSK 269962 | ROCK inhibitor |
| RKI-1447 | ROCK inhibitor |
| Lexibulin (CYT997) | Mt depolymerization |
| NPI-2358 (Plinabulin) | Mt depolymerization |
| Epothilone A | Mt stabilization similar to taxanes |
| Nocodazole | Mt depolymerization |
| Vincristine | Mt depolymerization |
| SCH 79797 | PARI antagonist |
| Lenalidomide | Transcriptional regulation |
| Gemcitabine | nucleoside analog |
| Domperidone (Motilium) | dopamine receptor antagonis |
| Puerarin (Kakonein) | 5-HT antagonist |
| Dioscin (Collettiside III) | Saponin |
| Indole-3-carbinol | food-supplement |
| Berberine Hydrochloride | plant alcaloid |

TABLE 10

| | |
|---|---|
| DNA damage | |
| Mitoxantrone | DNA intercalating |
| Hydrochloride | agent |
| Daunorubicin HCl | DNA intercalating |
| (Daunomycin HCl) | agent |
| Epirubicin | DNA intercalating |
| Hydrochloride | agent |
| Idarubicin | DNA intercalating |
| HCl | agent |
| Topotecan | topoisomerase |
| HCl | inhibitor |
| (S)-(+)- | Topoisomerase |
| Camptothecin | inhibitor |
| 10- | Topoisomerase |
| Hydroxycamptothecin | inhibitor |
| AZD2461 | PARP inhibitor |
| Mycophenolic acid | inhibitor of purine synthesis |
| Cell cycle arrest | |
| Flavopiridol hydrochloride | CDK inhibitor |
| Dinaciclib (SCH727965) | CDK inhibitor |
| SNS-032 (BMS-387032) | CDK-2 inhibitor |
| AT7519 | multi-CDK inhibitor |
| Abemaciclib (LY2835219) | CDK4/6 inhibitor |
| CHIR-124 | Chk1 inhibitor |
| NH125 | eEF-2 kinase inhibitor |
| Tyrosin kinases inhibitor | |
| VX-702 | p38 MAPK inhibitor |
| CP 673451 | PDGFR inhibitor |
| PRT062607 | Syk inhibitor |
| (P505-15,) HCl | |
| JAK3 Inhibitor VI | |
| GDC-0879 | Raf and ERK inhibitor |
| NF-kB inhibitors | |
| Triptolide | NF-κB inhibitor |
| Bardoxolone Methyl | IκB kinase/NF-kB inhibitor |
| A-674563 | Akt1 inhibitor |
| PIK-75 | PI3K inhibitor |
| GSK2126458 | PI3K inhibitor |
| RITA (NSC 652287) | MDM2 inhibitor |
| Retinoic acid derivates | |
| Acitretin | Retinoic acid derivate |
| Tazarotene (Avage) | Retinoic acid derivate |
| Bexarotene | Retinoic acid derivate |
| All-trans Retinoic Acid (Tretinoin) | Retinoic acid |
| Smooth muscle relaxants | |
| Tetrahydropapaverine hydrochloride | Phosphodiesterase (PDE) inhibitor |
| Icariin | PDE 5 inhibitor |
| Alverine Citrate | Ca++ channel blocker |
| 5-hydroxymethyl tolterodine | muscarinic receptor antagonist |

Others (pharmacologically active)

| | |
|---|---|
| Salicin (Salicoside) | COX inhibitor |
| Lorcaserin HCl | 5-HT (Serotonin) Receptor agonist |
| Vortioxetine hydrobromide | 5-HT Receptor agonist/SSRI |
| L-Thyroxine | thyroid hormone |
| Carbimazole | thyroid peroxidase inhibitor |
| Tetrandrine (Fanchinine) | Ca++ channel blocker |
| Shikimic acid (Shikimate) | precursor af aromatic amino acids |
| Ketoconazole | antifungal drug |
| Enrofloxacin | bacterial topoisomerase inhibitor |

Poorly characterised/plant-derived/food supplements

| | |
|---|---|
| Apocynin (Acetovanillone) | NADPH oxidase inhibitor |
| Naringin | Flavonoid, P450 inhibitor, |
| (Naringoside) | antioxidant |
| Quercetin dihydrate | Flavonoid, |
| (Sophoretin) | antioxidant |
| Chrysin | Flavonoid |
| Kaempferol | Flavonoid |
| Sesamin (Fagarol) | Food supplement |
| Vanillylacetone | Antioxidant |

Inhibition of TJ Disruption, by TJ Stabilizers

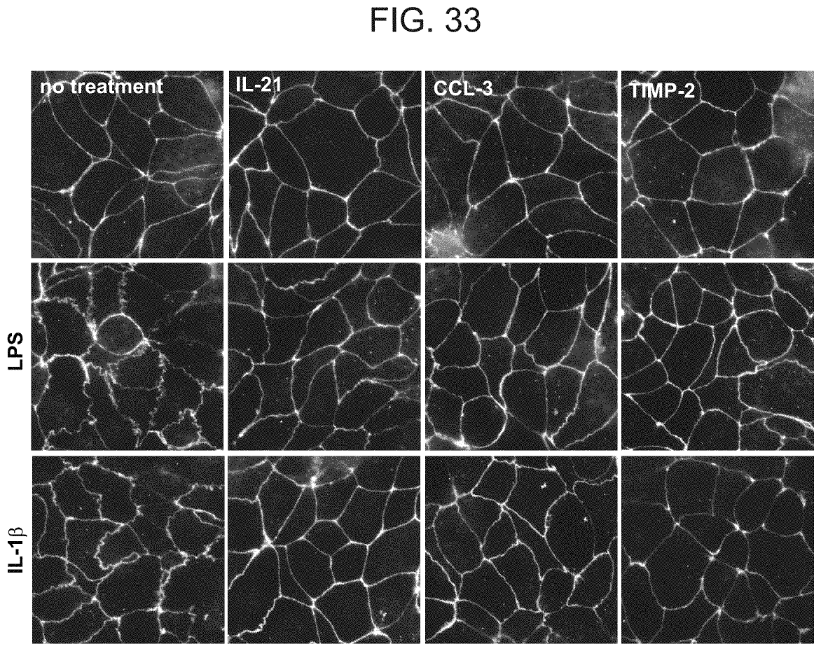

Caco2 cells were treated with ether single disrupting or stabilizing agent or with their combination, fixed and labeled for cingulin. In all cases combination of newly identified stabilizers with disrupting agents abolished distortion of tight junctions observed in the treatment with disruptors alone—FIG. 33.

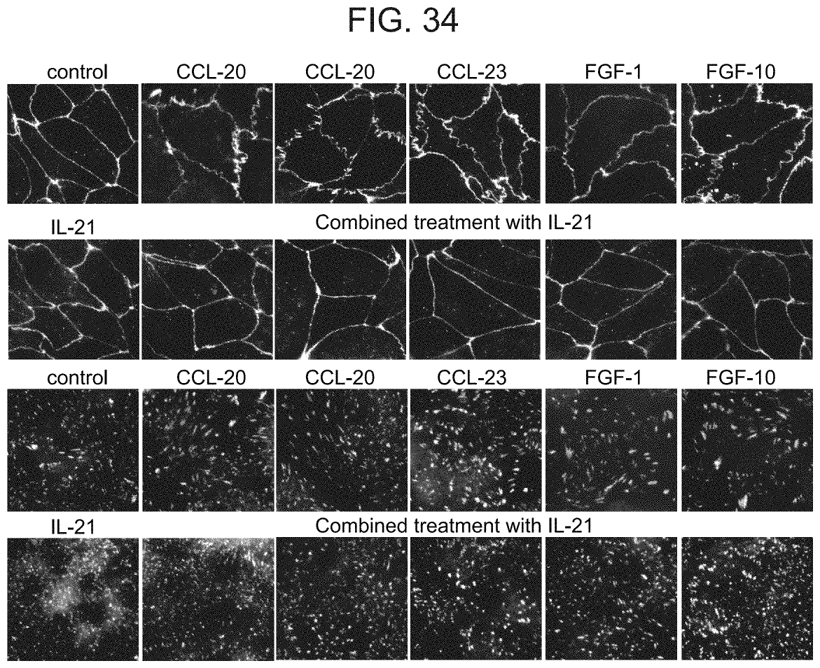

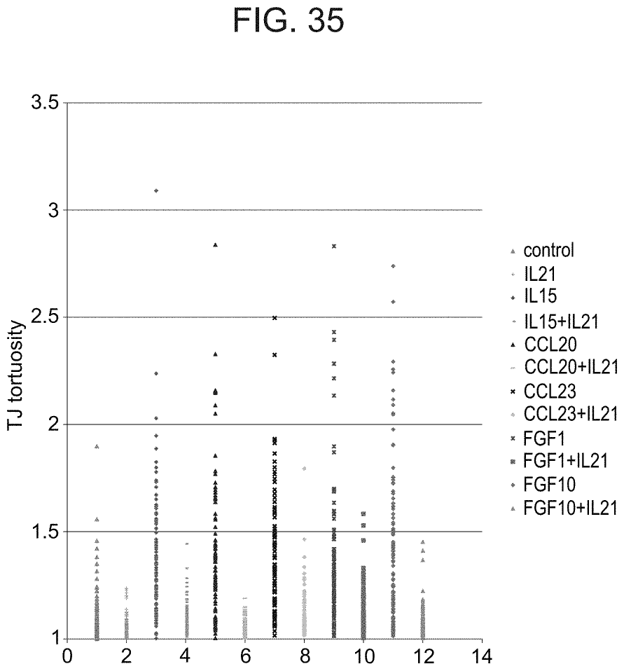

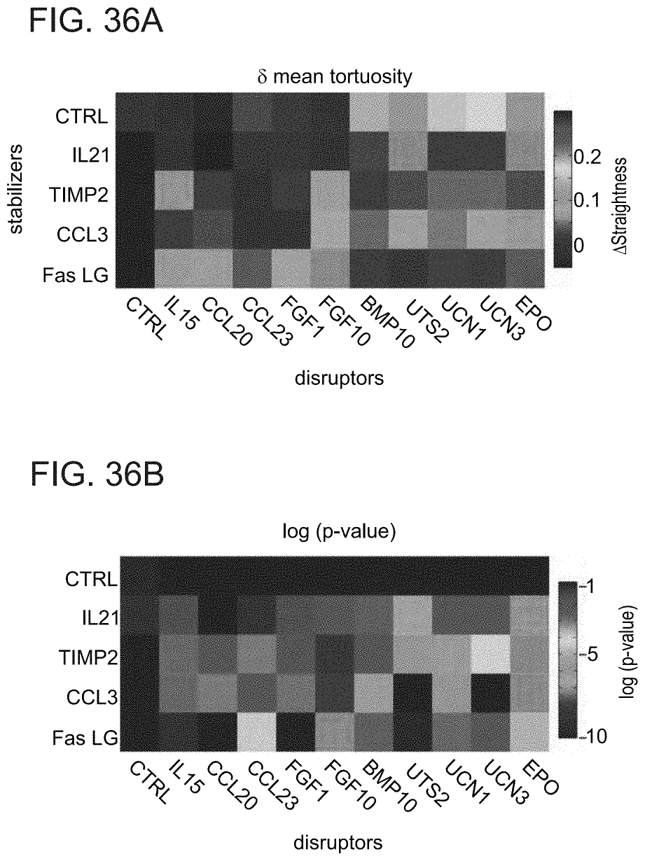

Caco2 cells were treated with a disruptor or with IL-21 or with a combination of disruptor plus IL-21, and labeled for cingulin and paxilin. As illustrated in FIG. 34, upper 2 panels show tight junctions and bottom 2 panels show focal adhesion in the same fields of view. The combination of disrupting agents with IL-21 abolished distortion of tight junctions observed in the treatment with disruptors alone. In addition co-treatment with IL-21 also prevents enlargement of focal adhesions caused by treatments with disruptors only. FIGS. 35 and 36A-B quantitate the level of disruption and stabilization by each of the agents.

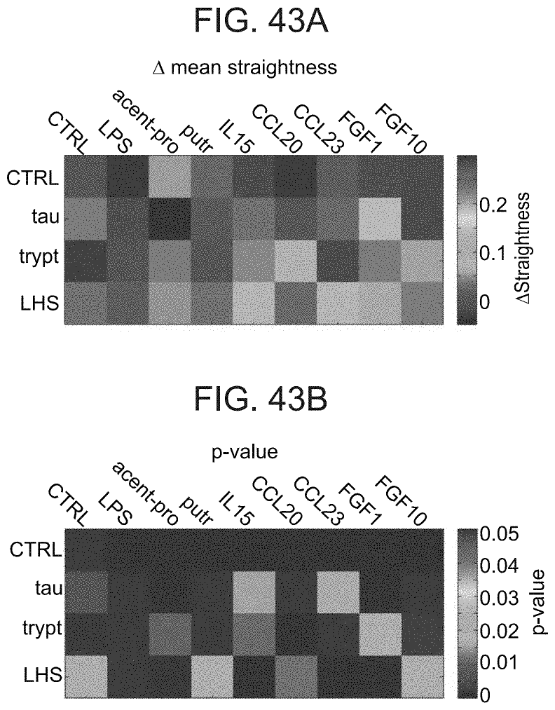

The effect of stabilizing metabolites against disruptive metabolites and selected secreted molecules is illustrated in FIG. 43A-B.

Reversibility of Tight Junction (TJ) Disruption Effect, in the Presence or Absence of Stabilizers:

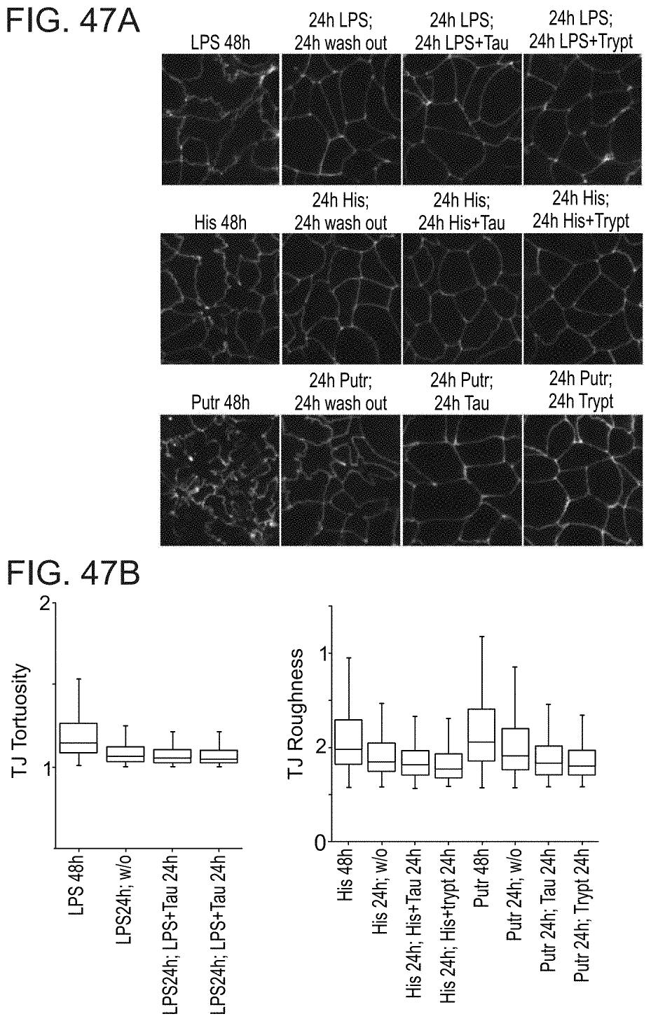

To determine the mode and time scale of TJ disruption, and the capacity of CaCo2 cells to restore normal TJ structure, CaCo2 cells were treated with 3 disruptors, cultured under the conditions described in the materials and methods, with 3 ‘disruptors’ (LPS (300 ng/ml), Histamine (10 mM) and putrescine (1 mM) for 24 hrs. This treatment, resulted in pronounced TJ disruption (FIG. 47A). Replacement of the disruptors by regular culture medium for 24 hours (‘washout’) resulted in nearly complete recovery of the TJs, in cells pretreated with LPS or histamine, and partial recovery following putrescine pretreatment (second panels from left). Replacement of the three disruptors with stabilizers (taurine, third panels from left, or tryptamine, fourth panels from left) even in the presence of the particular disruptors, induced full recovery of TJ structure.

Acto-Myosin Contractility Inhibitors as Potential TJ Stabilizers

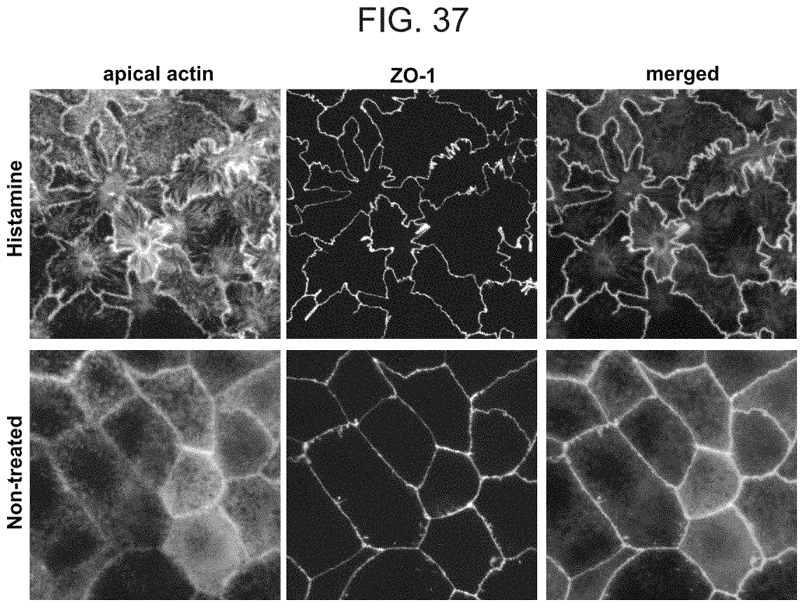

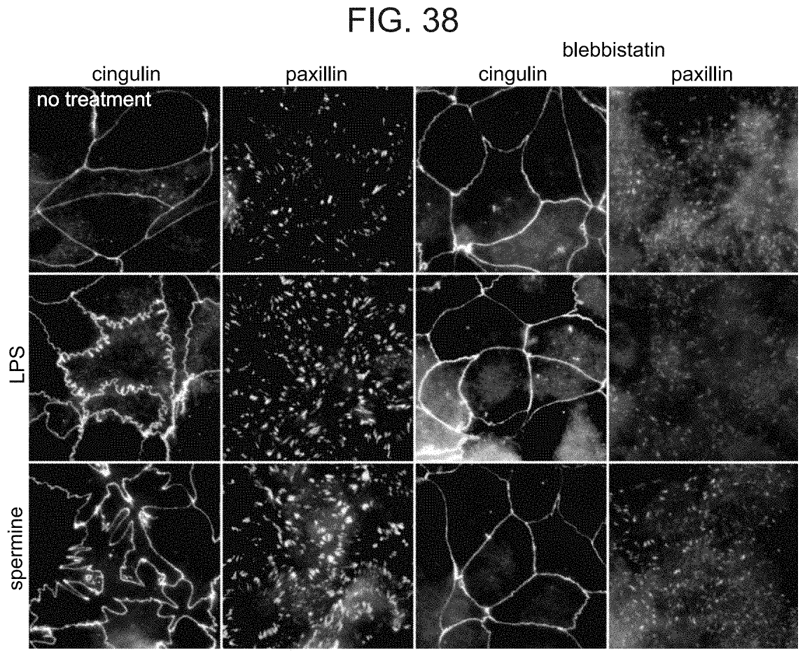

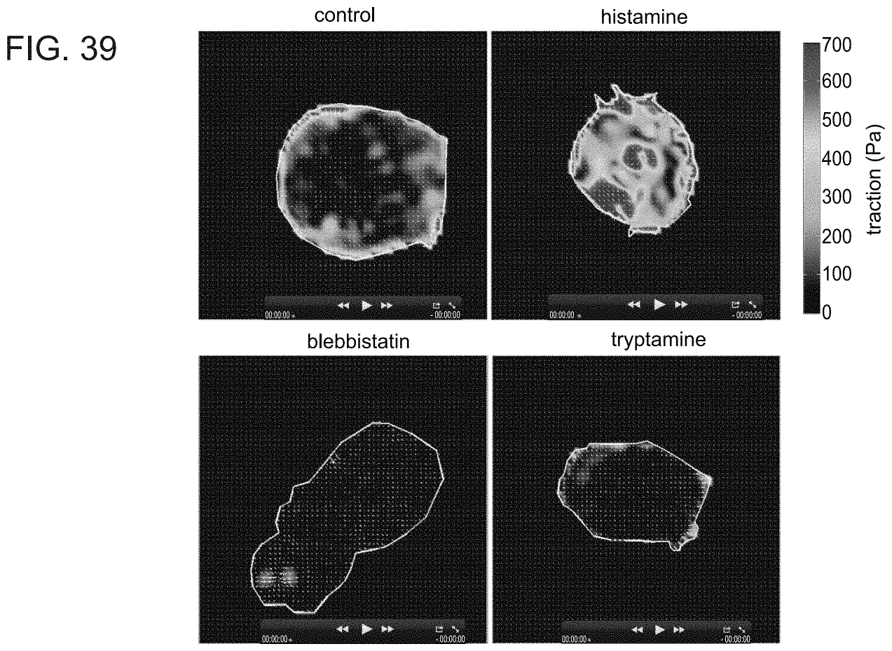

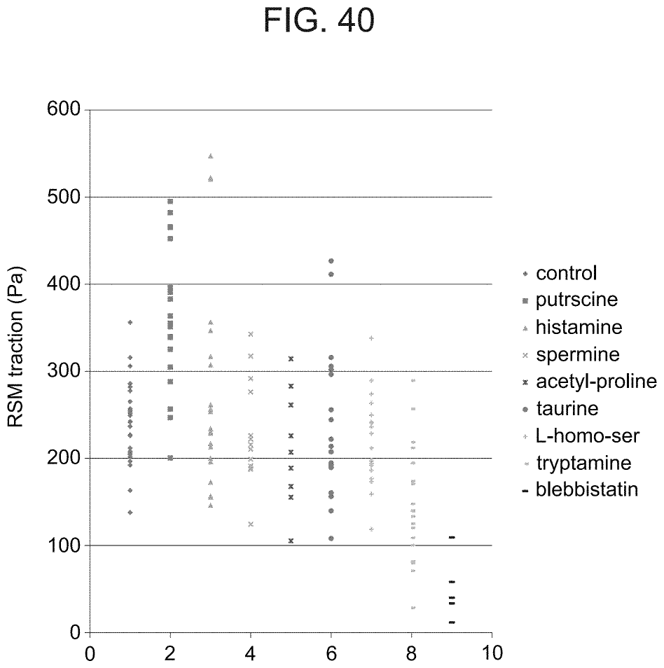

Frequently observed inverse correlation between TJ and FA phenotypes suggests involvement of acto-myosin contractile machinery into TJ regulation—see FIGS. 37-40.

Disrupting Activity of Fecal Extract from Murine IBD Model, and Blocking of the Disruption by Fecal Extract from a Healthy Animal.

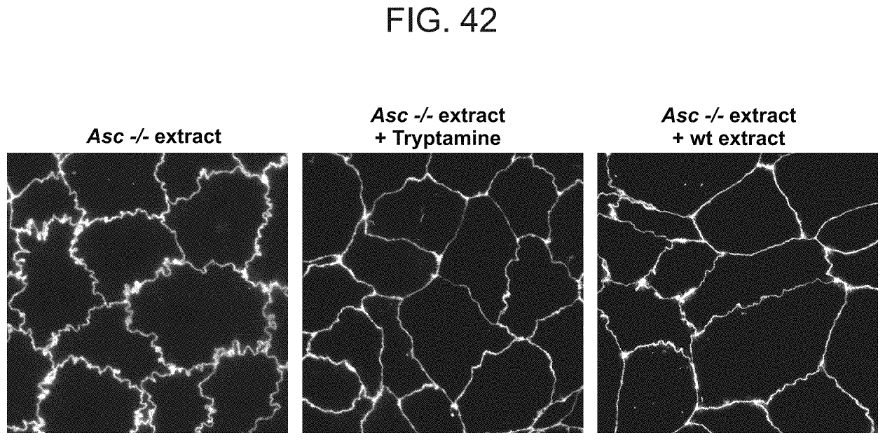

FIG. 41 indicates that fecal extract from a healthy mouse is capable of restoring epithelial disruption caused by bacterial metabolites or antigens. FIG. 42 indicates that fecal extract from a healthy mouse can block the detrimental effect of fecal extract derived from diseased mouse acts on TJ integrity.

### Example 3

### In Vivo Corroboration of the Disruptor Activity and Stabilizing Activity of the Agents Uncovered Using the In-Vitro Assay

Materials and Methods

Mice:

Wild-type C57Bl/6 male mice were purchased from Envigo and acclimatized to the animal facility environment for 2 weeks before experiments. All mice were kept under strict 12-h light-dark cycle (lights on at 6 am off at 6 pm). In ex vivo colon organ culture experiment, 2-week-old infant mice were used. Mice were 8-9 weeks of age when used for all in vivo experiments.

For putrescine treatment, mice received putrescine by oral gavage with a dose of 200 mg/kg twice daily for 10 days prior to and during DSS treatment or Citrobacter rodentium infection. For taurine treatment, mice received 30 mg/ml taurine in their drinking water for 10 days prior to and during infection models.

Ex-Vivo Intestinal Organ Culture System:

A three-dimensional device was used for gut organ culture and experiment, which was previously described in detail (Yissachar et al., Mar. 9, 2017, Cell 168, 1135-1148). Briefly, intact whole colons from 12- to 14-day-old SPF mice were dissected sterilely. The solid contents in the colon lumen were flushed gently. The colon fragment was then threaded with a sterile surgical thread and fixed across the luminal input and output ports. The gut culture device was maintained at 37° C. on a controlled heating block, and colon tissues were half-soaked in sterile medium at a constant flow. The tissue culture medium, containing different concentrations of putrescine, was loaded into a 10 ml syringe and continuously infused into the device input ports by a syringe pump (flow rate of 1 ml/h). Medical degrade 5% CO₂ and 95% O₂ gas mixture is provided to the device from a compressed gas cylinder connected to a regulator. After culturing for 2 hours, colon tissues were harvested, fixed in 4% paraformaldehyde, paraffin-embedded and sectioned for HE staining and immunofluorescence staining.

DSS-Induced Colitis:

Mice were administered with 2% (w/v) dextran sulfate sodium (DSS, M.W.=36,000-50,000 Da; MP Biomedicals) in their drinking water for 5 or 7 days followed by regular water. The animals were weighed daily, monitored for signs of distress and rectal bleeding. Colonoscopy was performed on day 7 using a high-resolution mouse video endoscopic system (Carl Storz, Tuttlingen, Germany). The severity of colitis was scored blindly using MEICS (Murine Endoscopic Index of Colitis Severity), which consists of five parameters: granularity of mucosal surface; vascular pattern; translucency of the colon mucosa; visible fibrin; and stool consistency (Becker et al., Nat Protoc. 2006; 1(6):2900-4). Mice were sacrificed for measurement of colon length and histological analysis on day 10. Pathological severity was scored by a pathologist in a blind manner, based on the degree of inflammation (location and extent), edema, mucosal ulceration, hyperplasia, crypt loss or abscess (Elinav et al., Cell. 2011 May 27; 145(5): 745-757).

Citrobacter rodentium Infection:

A kanamycin-resistant and luciferase-expressing derivative of Citrobacter rodentium DBS100 (ICC180) was used for infection as previously described (Thaiss et al., Science. 2018 Mar. 23; 359(6382):1376-1383).

Mice were infected with 200 μl solution containing approximately 1×10⁹ colony-forming units (CFU) of overnight cultured bacteria by oral gavage. Infection was monitored by enumerating stool CFU and measuring abdominal bioluminescent. For in vivo bioluminescent imaging, mice were anesthetized and bioluminescence was measured using an IVIS2000 instrument and Living Image software (Perkin Elmer). For ex vivo luminescence quantification, the whole colons were dissected, extensively washed from all luminal contents, longitudinally cut open and immediately imaged. For CFU counts, stool or tissues were collected, weighed and homogenized in sterile phosphate buffered saline (PBS−/−). Homogenates were then serially diluted in PBS−/− and plated on LB kanamycin plates, incubated overnight at 37° C. Bacterial CFUs were counted the next day, normalizing them to the stool or tissue weight.

Measuring Intestinal Permeability by FITC-Dextran:

On the day of FITC-dextran assay, 4 kDa fluorescein isothiocyanate (FITC)-dextran (Sigma, FD4) was dissolved in PBS−/− to a concentration of 80 mg/ml. Mice were fasted for 4 hours prior to gavage with 200 μl dextran. Mice were anesthetized 3 hours following gavage and blood was collected and centrifuged at 10,000×g for 12 min at 4° C. Serum was collected and fluorescence was quantified at an excitation wavelength of 485 nm and an emission wavelength of 535 nm.

Ussing Chamber:

An Ussing chamber system (Warner Instruments P2300) was used to measure transport across colon epithelial membranes according to the manufacturer's instructions. Briefly, after calibration of EasyMount chambers, colonic tissue was dissected from mice, gently washed, cut open and immediately mounted on the EasyMount Inserts. Voltage clamp modes were performed and short-circuit current as well as transepithelial electrical resistance were recorded. Colon tissues were kept at 37° C. in the chambers in physiological salt solution throughout the duration of the recording.

Quantification of Microbial Products in Organs:

A series of pattern recognition receptor (PRR) reporter cell lines (Invivogen, HEK-Blue TLR and NLR reporter cell lines) were used for quantification of microbial products, including TLR2, TLR3, TLR4, TLR5, TLR7, TLR9, NOD1, NOD2. Lymph node, spleen, and liver were harvested from mice, homogenized in PBS−/−, and added to reporter cell lines incubated with HEK-Blue Detection medium (Invivogen) according to the manufacturer's instructions. Secreted embryonic alkaline phosphatase (SEAP) activity was assessed by reading the optical density at 620-655 nm with a microplate reader.

Immunofluorescence Staining:

Colon samples were dissected, extensively washed, fixed in 4% paraformaldehyde, paraffin-embedded and sectioned. Sections were deparaffinized and subsequently antigen retrieved in 10 mM citric acid, pH 6. Slides were blocked with PBS−/− containing 20% (v/v) normal horse serum and 0.2% (v/v) Triton X-100 for 1 hour, and then incubated with rabbit anti-ZO-1 primary antibody (Invitrogen 40-2200) at 4° C. overnight. Sections were washed and incubated for 1 hour at room temperature with Alexa 488-conjugated donkey anti rabbit secondary antibody. Alternatively, sections were stained with Ki67 to assess proliferating cells or anti-cleaved caspase3 antibody to indicate apoptotic cells.

Flow Cytometry:

Colons were dissected from mice, extensively washed from fecal matter followed by 2 mM EDTA dissociation in 37° C. for 20 min. Following extensive shaking, the epithelial fraction was discarded and colons were then digested using DNAaseI and Collagenase for lamina propria analysis. The isolated cells were washed with cold PBS and resuspended in PBS containing 1% BSA. For cell surface staining, single-cell suspensions were blocked and stained with antibodies against CD45, CD3, CD4 and RORgt for 30 min on ice. For cytokine staining, cells were first incubated in restimulation medium for 2 h at 37° C. and then stained with antibodies against IL-17A, IFNg, IL-22 for 30 min on ice. Stained cells were analyzed on a BD-LSR Fortessa cytometer and were analyzed with FlowJo software.

Statistical Analysis:

Data were expressed as mean±SEM. P values <0.05 were considered significant (*p<0.05; **p<0.01; ***p<0.001; ****p<0.0001). Pairwise comparisons were performed using Student's t test. Mann-Whitney U test was used when data was not normally distributed.

Results

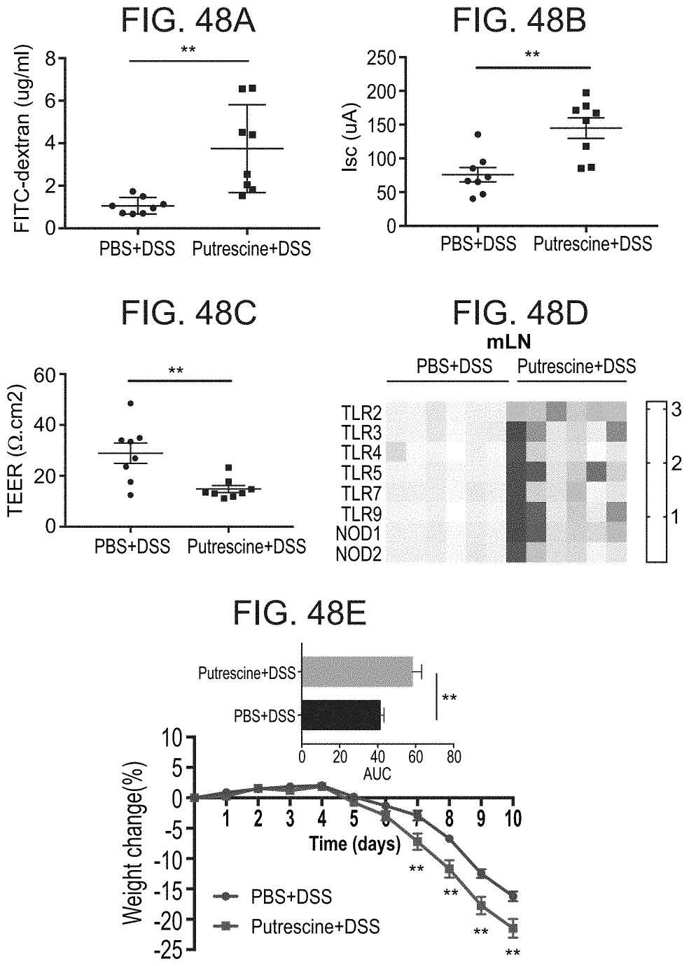

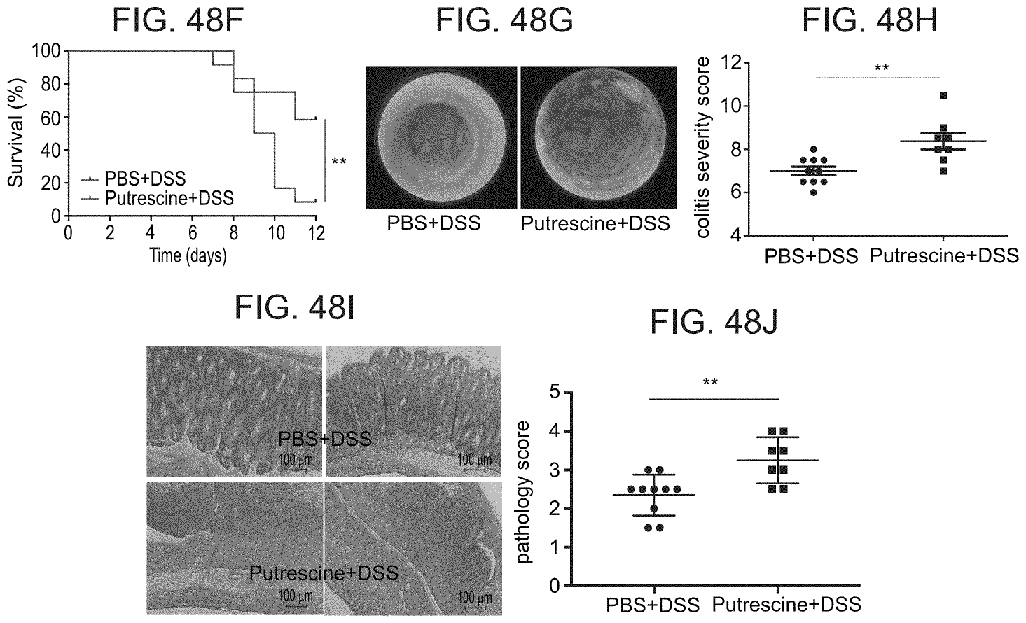

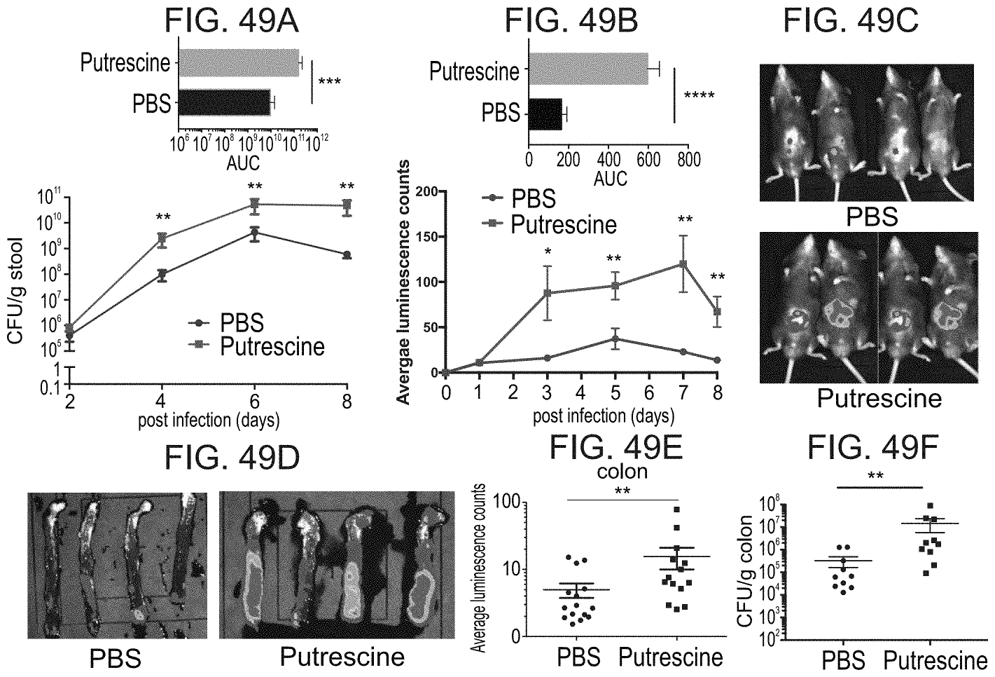

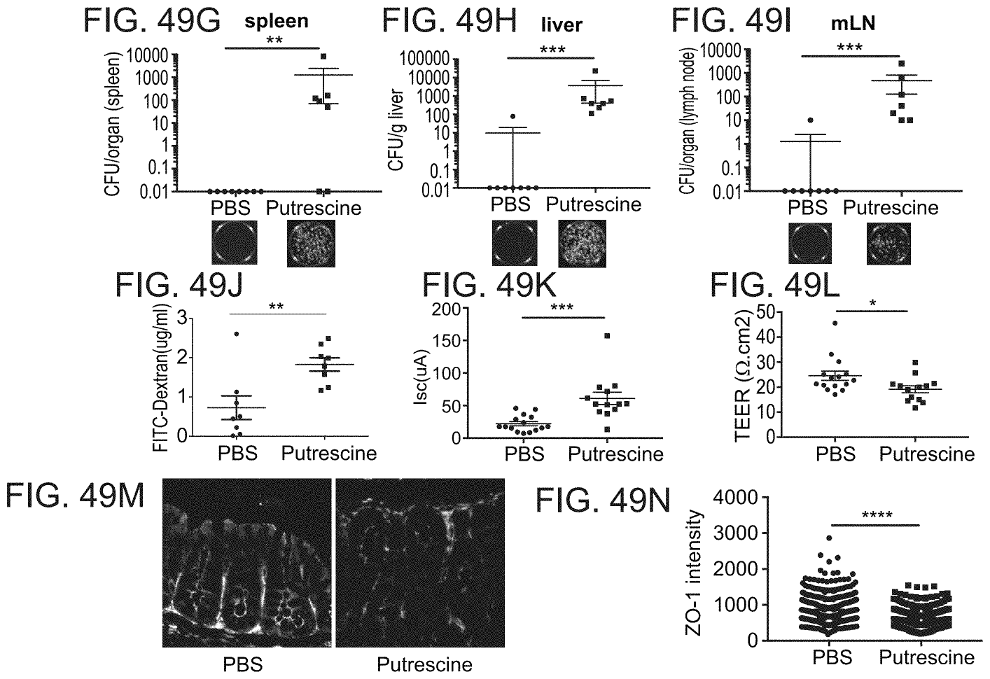

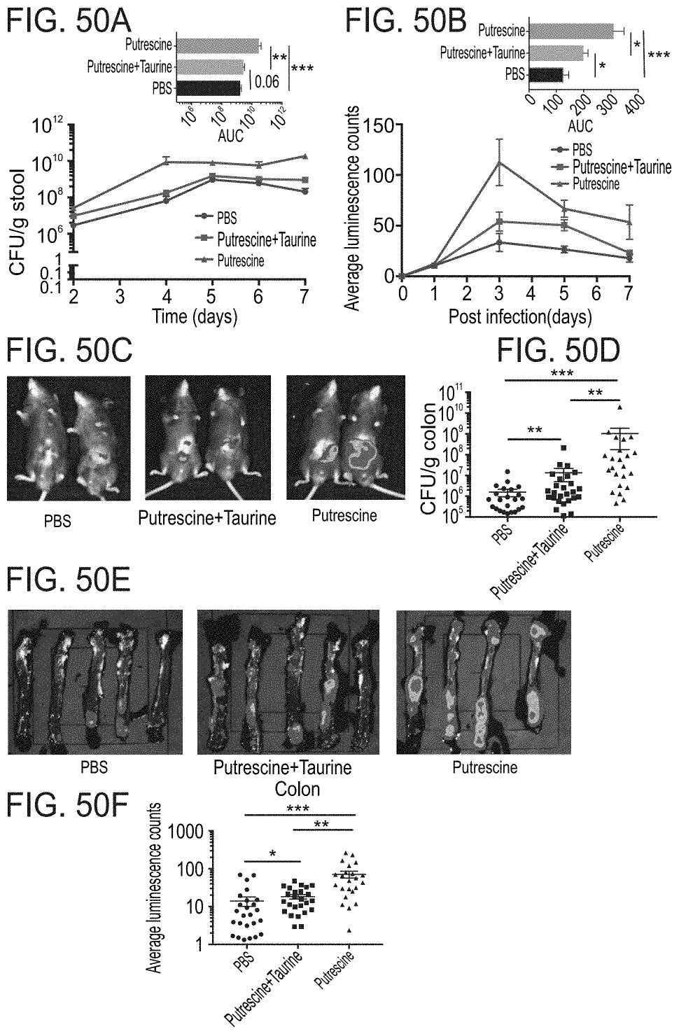

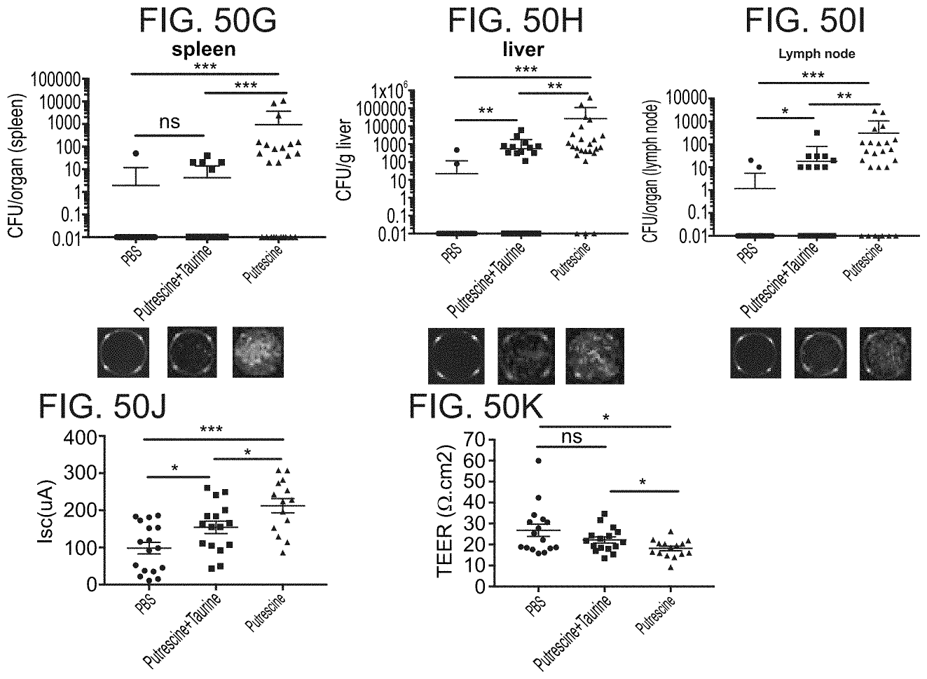

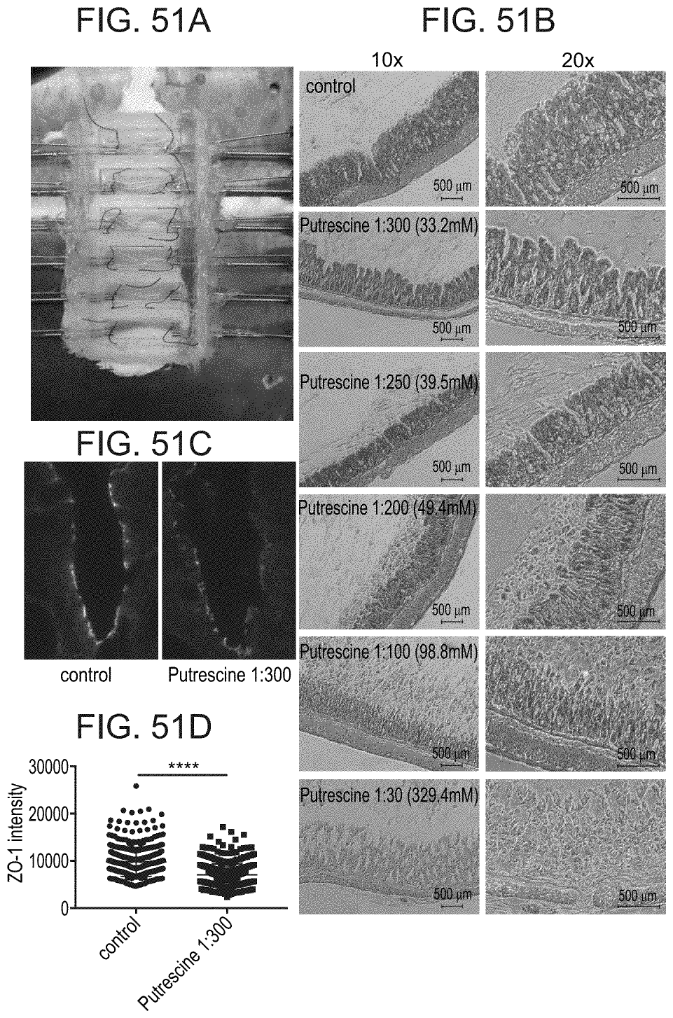

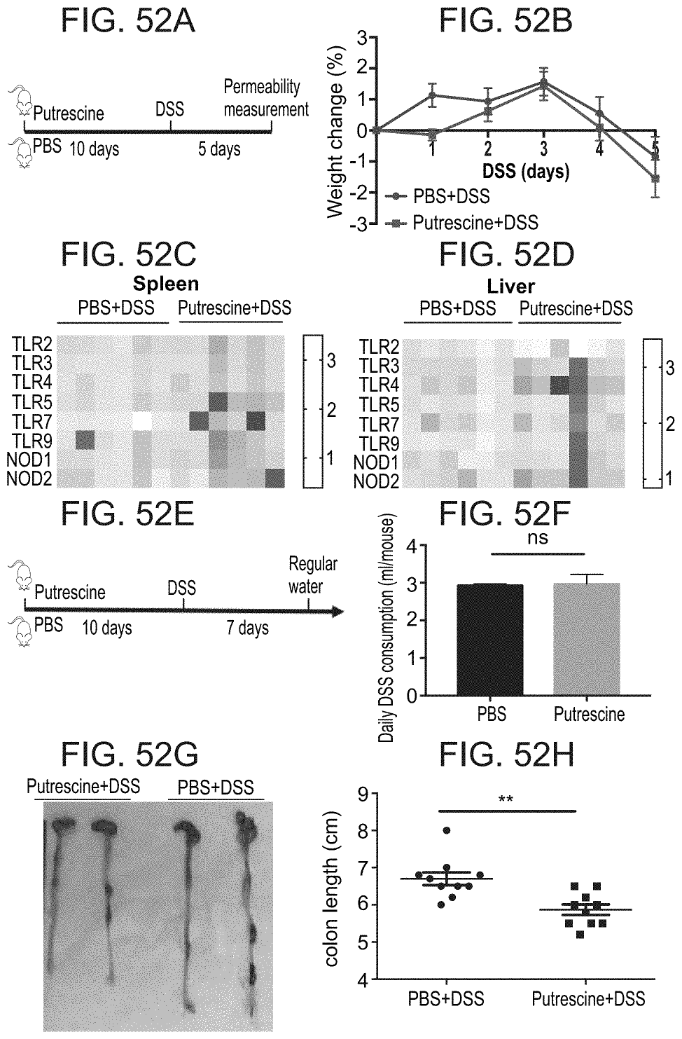

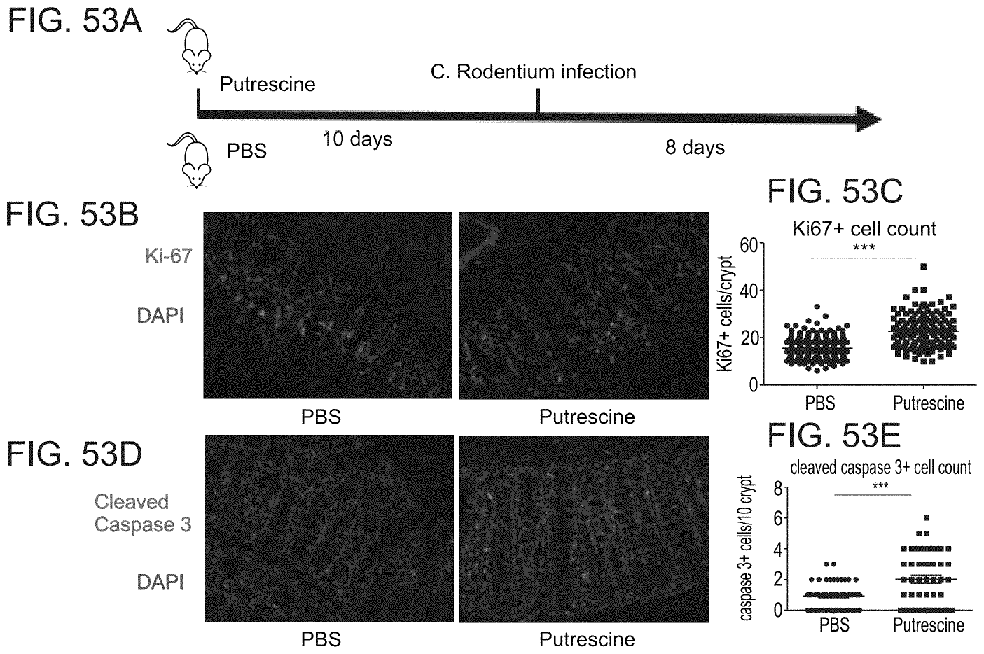

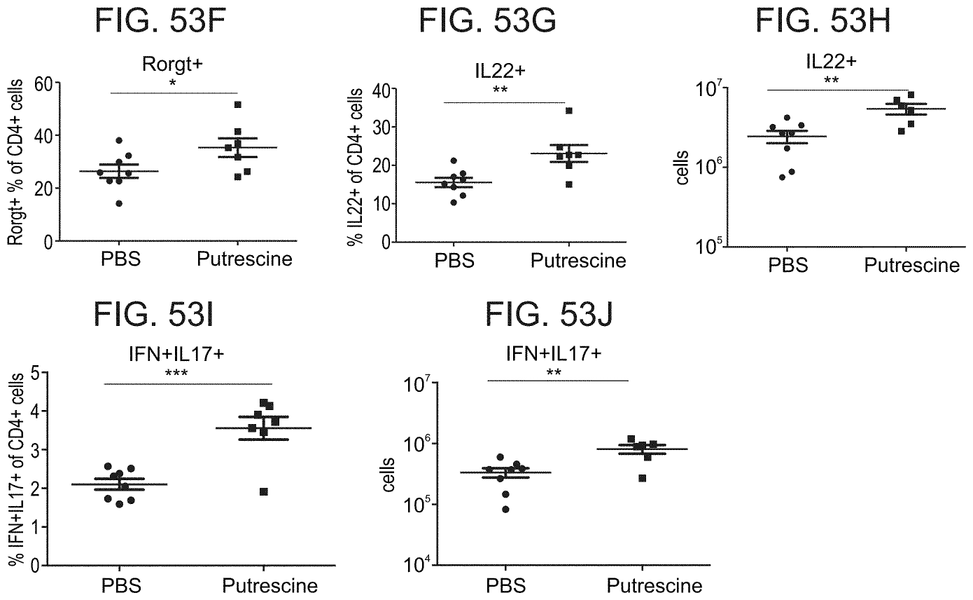

FIGS. 48A-J and 52A-H illustrate the disruptive effect of putrescine in mice with DSS-induced colitis. FIGS. 49A-N and 53A-J illustrate the disruptive effect of putrescine in mice with C. rodentium infection. FIGS. 50A-K illustrate restoration of the disruptive effect of putrescine by taurine supplement in mice with C. rodentium infection. FIGS. 51A-D illustrate the effect of putrescine on TJ integrity in an intestinal organ culture system.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority documents of this application are hereby incorporated herein by reference in its/their entirety.

What is claimed is:

1. A method of identifying agents useful for treating a disease associated with intestinal barrier dysfunction of a subject, the method comprising:
(a) contacting tight junctions of epithelial cells with a disruptor agent that promotes disruption or destabilization of tight junctions of epithelial cells; and
(b) contacting said tight junctions of epithelial cells with a test agent; and
(c) analyzing the effect of said test agent on said tight junctions of epithelial cells, wherein when said test agent prevents disruption of, or stabilizes said tight junctions, it is indicative of said test agent being useful for treating the disease associated with intestinal barrier dysfunction of the subject.

2. The method of claim 1, wherein said agent that promotes disruption or destabilization is a sample derived from the gastrointestinal tract (GIT) of the subject.

3. The method of claim 1, wherein said agent which promotes disruption or destabilization of tight junctions of epithelial cells is set forth in Tables 5, 7 or 9.

4. The method of claim 1, wherein said epithelial cells are selected from the group consisting of CaCo-2 cells, DLD-1 cells, HT-29 cells, T-84 cells and LoVo cells.

5. The method of claim 1, wherein said epithelial cells are derived from the subject.

6. A method of treating a disease associated with intestinal barrier dysfunction in a subject in need thereof, the method comprising:
(a) identifying an agent useful for treating the disease according to claim 1; and
(b) administering to the subject a therapeutically effective amount of said agent, thereby treating the disease associated with intestinal barrier dysfunction.

7. A method of treating a disease associated with intestinal barrier dysfunction in a subject in need thereof, the method comprising administering to the subject a therapeutically effective amount of an agent which increases the amount and/or activity of at least one agent set forth in Table 2, thereby treating the disease associated with intestinal barrier dysfunction.

8. A method of treating a disease associated with intestinal barrier dysfunction in a subject in need thereof, the method comprising administering to the subject a therapeutically effective amount of an agent which decreases the amount and/or activity of at least one agent set forth in Table 3, thereby treating the disease associated with intestinal barrier dysfunction.

9. The method of claim 7, wherein said disease associated with barrier dysfunction is selected from the group consisting of Non Alcoholic Fatty Liver Disease (NAFLD); Non alcoholic steatohepatitis (NASH); Pre-diabetes and glucose intolerance.

10. The method of claim 7, wherein the disease associated with intestinal barrier dysfunction is a disease associated with systemic inflammation.

11. The method of claim 10, wherein said disease associated with systemic inflammation is selected from the group consisting of cancer, aging and neurodegeneration.

12. The method of claim 7, wherein said disease is a disease of the GIT.

13. The method of claim 12, wherein said disease of the GIT is selected from the group consisting of inflammatory bowel disease, metabolic syndrome, gut infection and gut autoinflammation.

14. A co-culture system comprising cells of a tissue derived from a subject and microbes derived from a microbiome of said tissue of said subject.

15. The method of claim 8, wherein said disease associated with barrier dysfunction is selected from the group consisting of Non Alcoholic Fatty Liver Disease (NAFLD); Non alcoholic steatohepatitis (NASH); Pre-diabetes and glucose intolerance.

16. The method of claim 8, wherein the disease associated with intestinal barrier dysfunction is a disease associated with systemic inflammation.

17. The method of claim 16, wherein said disease associated with systemic inflammation is selected from the group consisting of cancer, aging and neurodegeneration.

18. The method of claim 8, wherein said disease is a disease of the GIT.

19. The method of claim 18, wherein said disease of the GIT is selected from the group consisting of inflammatory bowel disease, metabolic syndrome, gut infection and gut autoinflammation.


SEQUENCE LISTING (2 sequences)

SEQ ID NO: 1 - DNA, 19 bp, Artificial Sequence
  Single strand DNA oligonucleotide
gtgccagcmgccgcggtaa

SEQ ID NO: 2 - DNA, 20 bp, Artificial Sequence
  Single strand DNA oligonucleotide
ggactachvgggtwtctaat